(19) United States
(12) Patent Application Publication
(10) Pub. No.: US 2024/0254168 A1
(43) Pub. Date: Aug. 1, 2024

(54) LIPID PARTICLES CONTAINING A TRUNCATED BABOON ENDOGENOUS RETROVIRUS (BaEV) ENVELOPE GLYCOPROTEIN AND RELATED METHODS AND USES

(71) Applicant: Sana Biotechnology, Inc., Seattle, WA (US)

(72) Inventors: Richard C. Mulligan, Cambridge, MA (US); Michael J. VOLLES, Cambridge, MA (US)

(73) Assignee: Sana Biotechnology, Inc., Seattle, WA (US)

(21) Appl. No.: 18/563,812

(22) PCT Filed: May 27, 2022
(86) PCT No.: PCT/US2022/031459

§ 371 Date: Nov. 22, 2023

Related U.S. Application Data
(60) Provisional application No. 63/194,880, filed on May 28, 2021.

(51) Int. Cl. C07K 14/155; C12N 15/86

(52) U.S. Cl. CPC C07K 14/155; C12N 15/86; C12N 2740/10022; C12N 2740/15043; C12N 2830/48; C12N 2830/50

(57) ABSTRACT

Provided herein are lipid particles, such as lentiviral particles, that incorporate or are pseudotyped with a truncated Baboon Endogenous Retrovirus (BaEV) envelope glycoprotein that contains a cytoplasmic tail with a partial inhibitory R peptide that is less than the full length wildtype BaEV inhibitory R peptide. Also provided herein are polynucleotides encoding the truncated BaEV envelope glycoproteins and producer cells for preparation of the lipid particles, such as lentiviral particles, containing the truncated BaEV envelope glycoproteins, as well as methods for preparing and using the lipid particles, such as lentiviral particles.

### CROSS REFERENCE TO RELATED APPLICATIONS

This application claims priority to U.S. provisional application No. 63/194,880 filed May 28, 2021, entitled “LIPID PARTICLES CONTAINING A TRUNCATED BABOON ENDOGENOUS RETROVIRUS (BaEV) ENVELOPE GLYCOPROTEIN AND RELATED METHODS AND USES”, the contents of which are hereby incorporated by reference in their entirety for all purposes.

### INCORPORATION BY REFERENCE OF SEQUENCE LISTING

The present application is being filed with a Sequence Listing in electronic format. The Sequence Listing is provided as a filed entitled “18615_2004440_SEQLIST”, created May 27, 2022, which is 138,255 bytes in size. The information in the electronic format of the Sequence Listing is hereby incorporated by reference in its entirety.

### FIELD

The present disclosure relates to lipid particles, such as lentiviral particles, that incorporate or are pseudotyped with a truncated Baboon Endogenous Retrovirus (BaEV) envelope glycoprotein that contains a cytoplasmic tail with a partial inhibitory R peptide that is less than the full length wild-type BaEV inhibitory R peptide. The present disclosure also provides polynucleotides encoding the truncated BaEV envelope glycoproteins and producer cells for preparation of the lipid particles, such as lentiviral particles, containing the truncated BaEV envelope glycoproteins, as well as methods for preparing and using the lipid particles, such as lentiviral particles.

### SUMMARY

Lipid particles, including virus-like particles and viral vectors such as lentiviral particles, are commonly used for delivery of exogenous agents to cells. For various particles, such as lentiviral vector particles, the host range can be altered by pseudotyping with a heterologous envelope protein. The efficient preparation and production of particles with certain heterologous pseudotyped envelope proteins may not always be efficient, such as due to effects of the envelope protein on low titer of the produced lentiviral vector particles. Improved lipid particles, including virus-like particles and viral vectors, that are able to be produced with a higher titer and with efficient transduction efficiency of desirable cells are needed. The provided disclosure addresses this need.

Provided herein is a Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEv envelope glycoprotein

Also provided herein is a Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein.

In some of any of the provided embodiments, the lentiviral particle is replication defective. In some of any of the provided embodiments, the provided lentiviral particle is prepared by a method comprising transducing a producer cell with packaging plasmids that encode a Gag-pol, Rev, Tat and the truncated BaEV envelope glycoprotein.

In some of any of the provided embodiments, the lentiviral particle further comprises a viral nucleic acid. In some of any embodiments, the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3). In some of any embodiments, the lentiviral particle is devoid of viral genomic DNA.

Provided herein is a lipid particle comprising a truncated Baboon Endogenous Retrovirus (BaEV) envelope glycoprotein, comprising: (a) a lipid bilayer enclosing a lumen, and (b) a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one contiguous amino-terminal amino acids but less than the full length of the inhibitory R peptide of the inhibitory R peptide of the wild-type BaEV envelope glycoprotein, wherein the envelope glycoprotein is embedded in the lipid bilayer.

Provided herein is a lipid particle comprising a truncated Baboon Endogenous Retrovirus (BaEV) envelope glycoprotein, comprising: (a) a lipid bilayer enclosing a lumen, and (b) a truncated BaEV envelope glycoprotein comprising a cytoplasmic tail with a partial fusion inhibitory R peptide relative to the inhibitory R peptide of a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein, wherein the envelope glycoprotein is embedded in the lipid bilayer.

In some of any of the provided embodiments, the lipid bilayer is derived from a membrane of a host cell used for producing a retrovirus or retrovirus-like particle. In some of any embodiments, the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

In some of any of the provided embodiments, the lipid bilayer is or comprises one or more other viral components other than the BaEV envelope glycoprotein. In some of any of the provided embodiments, the one or more viral components are from a retrovirus. In some of any of the provided embodiments, the retrovirus is a lentivirus or a lentivirus like particle. In some of any of the provided embodiments, the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide. In some of any embodiments, the glycoprotein 70 (g70) subunit or a biologically active portion thereof, and the portion of the glycoprotein p20E (p20E) subunit are associated via an inter-subunit disulfide bond. In some of any embodiments, the BaEV glycoprotein binds an ASCT-2 or ASCT-1 receptor.

In some of any of the provided embodiments, the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25. In some of any of the provided embodiments, the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

In some of any of the provided embodiments, the truncated BaEV envelope glycoprotein is truncated relative to SEQ ID NO:24 and lacks up to 16 contiguous amino acids from the C-terminal cytoplasmic tail of SEQ ID NO:24. In some of any of the provided embodiments, the truncated BaEV envelope glycoprotein is truncated relative to SEQ ID NO:24 and lacks from 8 to 14, contiguous amino acids from the C-terminal cytoplasmic tail of SEQ ID NO:24, optionally from 8 to 13 contiguous amino acids from the C-terminal cytoplasmic tail of SEQ ID NO:24.

In some of any of the provided embodiments, the partial fusion inhibitory R peptide is set forth as amino acids 1 to 9 of SEQ ID NO:22, optionally wherein the cytoplasmic tail is set forth in SEQ ID NO:14 (R+9). In some of any of the provided embodiments, the truncated BaEV envelope glycoprotein is set forth in SEQ ID NO: 37. In some of any of the provided embodiments, the partial fusion inhibitory R peptide is set forth as amino acids 1 to 8 of SEQ ID NO:22, optionally wherein the cytoplasmic tail is set forth in SEQ ID NO:13 (R+8).

In some of any of the provided embodiments, the truncated BaEV envelope glycoprotein is set forth in SEQ ID NO: 36. In some of any of the provided embodiments, the partial fusion inhibitory R peptide is set forth as amino acids 1 to 7 of SEQ ID NO:22, optionally wherein the cytoplasmic tail is set forth in SEQ ID NO: 12 (R+7). In some of any of the provided embodiments, the truncated BaEV envelope glycoprotein is set forth in SEQ ID NO: 35. In some of any of the provided embodiments, the partial fusion inhibitory R peptide is set forth as amino acids 1 to 6 of SEQ ID NO:22, optionally wherein the cytoplasmic tail is set forth in SEQ ID NO:11 (R+6). In some of any of the provided embodiments, the truncated BaEV envelope glycoprotein is set forth in SEQ ID NO: 34. In some of any of the provided embodiments, the partial fusion inhibitory R peptide is set forth as amino acids 1 to 5 of SEQ ID NO:22, optionally wherein the cytoplasmic tail is set forth in SEQ ID NO:10 (R+5). In some of any of the provided embodiments, the truncated BaEV envelope glycoprotein is set forth in SEQ ID NO: 33. In some of any of the provided embodiments, the partial fusion inhibitory R peptide is set forth as amino acids 1 to 4 of SEQ ID NO:22, optionally wherein the cytoplasmic tail is set forth in SEQ ID NO:9 (R+4). In some of any of the provided embodiments, the truncated BaEV envelope glycoprotein is set forth in SEQ ID NO: 32. In some of any of the provided embodiments, the partial fusion inhibitory R peptide is set forth as amino acids 1 to 3 of SEQ ID NO:22, optionally wherein the cytoplasmic tail is set forth in SEQ ID NO:8 (R+3). In some of any of the provided embodiments, the truncated BaEV envelope glycoprotein is set forth in SEQ ID NO: 31.

In some of any of the provided embodiments, the particle further comprises an exogenous agent. In some of any embodiments, the exogenous agent is present in the lumen. In some of any of the provided embodiments, the exogenous agent is a protein or a nucleic acid, optionally wherein the nucleic acid is a DNA or RNA. In some of any of the provided embodiments, the particle is produced as a preparation with increased titer compared to a reference particle preparation that is similarly produced but with incorporation of a BaEV envelope glycoprotein having a cytoplasmic tail with a full length R peptide or a portion of the R peptide of 10 or more amino acids in length.

In some of any of the provided embodiments, the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more. In some of any of the provided embodiments, the titer in target cells, optionally HEK293 cells, following transduction is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL. In some of any of the provided embodiments, the truncated BaEV envelope glycoprotein is present on the surface of the particle at a density of at least about (0.001, 0.002, 0.005, 0.01, 0.02, 0.05, 0.1, 0.2 or 0.5) truncated BaEV envelope glycoproteins/nm².

Provided herein is a truncated BaEV envelope glycoprotein comprising a cytoplasmic tail with a partial fusion inhibitory R peptide relative to a wild-type BaEV envelope glycoprotein, wherein the partial fusion inhibitory R peptide comprises at least one contiguous amino-terminal amino acid, but less than the full length, of the inhibitory R peptide of the wild-type BaEV envelope glycoprotein.

In some of any of the provided embodiments, the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide. In some of any embodiments, the glycoprotein 70 (g70) subunit or a biologically active portion thereof, and the portion of the glycoprotein p20E (p20E) subunit are associated via an inter-subunit disulfide bond.

In some of any of the provided embodiments, the BaEV glycoprotein binds an ASCT-2 or ASCT-1 receptor.

In some of any of the provided embodiments, the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25. In some of any of the provided embodiments, the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

In some of any of the provided embodiments, the truncated BaEV envelope glycoprotein is truncated relative to SEQ ID NO:24 and lacks up to 16 contiguous amino acids from the C-terminal cytoplasmic tail of SEQ ID NO:24. In some of any embodiments, the truncated BaEV envelope glycoprotein is truncated relative to SEQ ID NO:24 and lacks from 8 to 14 contiguous amino acids from the C-terminal cytoplasmic tail of SEQ ID NO:24, optionally from 8 to 13 contiguous amino acids from the C-terminal cytoplasmic tail of SEQ ID NO:24. In some of any of the provided embodiments, the partial fusion inhibitory R peptide is set forth as amino acids 1 to 9 of SEQ ID NO:22, optionally wherein the cytoplasmic tail is set forth in SEQ ID NO: 14 (R+9). In some of any of the provided embodiments, the truncated BaEV envelope glycoprotein is set forth in SEQ ID NO: 37. In some of any of the provided embodiments, the partial fusion inhibitory R peptide is set forth as amino acids 1 to 8 of SEQ ID NO:22, optionally wherein the cytoplasmic tail is set forth in SEQ ID NO:13 (R+8). In some of any of the provided embodiments, the truncated BaEV envelope glycoprotein is set forth in SEQ ID NO: 36. In some of any of the provided embodiments, the partial fusion inhibitory R peptide is set forth as amino acids 1 to 7 of SEQ ID NO:22, optionally wherein the cytoplasmic tail is set forth in SEQ ID NO: 12 (R+7). In some of any of the provided embodiments, the truncated BaEV envelope glycoprotein is set forth in SEQ ID NO: 35. In some of any of the provided embodiments, the partial fusion inhibitory R peptide is set forth as amino acids 1 to 6 of SEQ ID NO:22, optionally wherein the cytoplasmic tail is set forth in SEQ ID NO:11 (R+6). In some of any of the provided embodiments, the truncated BaEV envelope glycoprotein is set forth in SEQ ID NO: 34. In some of any of the provided embodiments, the partial fusion inhibitory R peptide is set forth as amino acids 1 to 5 of SEQ ID NO:22, optionally wherein the cytoplasmic tail is set forth in SEQ ID NO:10 (R+5). In some of any of the provided embodiments, the truncated BaEV envelope glycoprotein is set forth in SEQ ID NO: 33. In some of any of the provided embodiments, the partial fusion inhibitory R peptide is set forth as amino acids 1 to 4 of SEQ ID NO:22, optionally wherein the cytoplasmic tail is set forth in SEQ ID NO:9 (R+4). In some of any of the provided embodiments, the truncated BaEV envelope glycoprotein is set forth in SEQ ID NO: 32. In some of any of the provided embodiments, the partial fusion inhibitory R peptide is set forth as amino acids 1 to 3 of SEQ ID NO:22, optionally wherein the cytoplasmic tail is set forth in SEQ ID NO:8 (R+3). In some of any of the provided embodiments, the truncated BaEV envelope glycoprotein is set forth in SEQ ID NO: 31.

Provided herein is a polynucleotide comprising a nucleic acid encoding the truncated BaEV envelope glycoprotein of any of the provided embodiments. In some of any of the provided embodiments, the polynucleotide is codon optimized.

In some of any of the provided embodiments, the polynucleotide further comprises at least one promoter that is operatively linked to control expression of the nucleic acid. In some of any embodiments, the promoter is a constitutive promoter. In some of any of the provided embodiments, the promoter is an inducible promoter.

Provided herein is a vector comprising any of the provided polynucleotides. Provided herein is a plasmid comprising any of the provided polynucleotides. In some of any of the provided embodiments, the plasmid further comprises one or more nucleic acids encoding proteins for lentivirus production.

Provided herein is a cell comprising any of the provided polynucleotides, vectors, or plasmids. In some of any of the provided embodiments, the cell is a producer cell for production of a lentiviral particle.

Provided herein is a producer cell comprising (i) a viral nucleic acid(s) and (ii) a nucleic acid encoding the truncated BaEV envelope glycoprotein of any of claims 43-64, optionally wherein the viral nucleic acid(s) are lentiviral nucleic acids. In some of any of the provided embodiments, the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In some of any of the provided embodiments, the producer cell comprises 293T cells.

In some of any of the provided embodiments, the viral nucleic acid(s) lacks one or more genes involved in viral replication. In some of any of the provided embodiments, the viral nucleic acid comprises a nucleic acid encoding a viral packaging protein selected from one or more of Gag, Pol, Rev and Tat. In some of any of the provided embodiments, the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

Provided herein is a method of making a lipid particle comprising a truncated BaEV glycoprotein comprising: a) introducing into a source cell any of the provided polynucleotides, any of the provided vectors, or any of the provided plasmids; b) culturing the cell under conditions that allow for production of a lipid particle, and c) separating, enriching, or purifying the lipid particle from the cell, thereby making the lipid particle.

In some of any of the provided embodiments, the source cell is a mammalian cell. In some of any of the provided embodiments, the source cell is a producer cell and the lipid particle is a viral particle or a viral-like particle, optionally a retroviral particle or a retroviral-like particle, optionally a lentiviral particle or lentiviral-like particle.

Provided herein is a method of making a pseudotyped lentiviral particle, the method comprising: a) providing a producer cell that comprises a lentiviral nucleic acid (s) and any of the provided nucleic acid encoding a truncated BaEV envelope glycoprotein of or any of the provided polynucleotides; b) culturing the cell under conditions that allow for production of the lentiviral particle, and c) separating, enriching, or purifying the lentiviral particle from the producer cell, thereby making the pseudotyped lentiviral particle.

In some of any of the provided embodiments, the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells. In some of any of the provided embodiments, the producer cell comprises 293T cells. In some of any of the provided embodiments, the method produces a lentiviral preparation with increased titer compared to a reference lentiviral particle preparation that is similarly produced but is pseudotyped with a BaEV envelope glycoprotein having a cytoplasmic tail with a full length R peptide or a portion of the R peptide of 10 or more amino acids in length.

In some of any of the provided embodiments, the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more. In some of any of the provided embodiments, the method produces a lentiviral preparation with a titer in target cells, optionally HEK293 cells, following transduction that is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL. In some of any of the provided embodiments, the method results in reduced syncytia formation of the producer cell compared to a similar method but that is for production of a reference lentiviral particle preparation pseudotyped with a BaEV envelope glycoprotein having a cytoplasmic tail with no R peptide (Rless) or an R peptide of 3 contiguous amino terminal amino acids or less in length relative to the wild-type BaEV envelope glycoprotein R peptide. In some of any of the provided embodiments, the method produces a lentiviral preparation with high titer (e.g. greater than 4×10⁶ TU/mL, greater than 5×10⁶ TU/mL, greater than 6×10⁶ TU/mL, greater than 7×10⁶ TU/mL, greater than 8×10⁶ TU/mL, greater than 9×10⁶ TU/mL, greater than 1×10⁷ TU/mL, or greater than 1.2×10⁷ TU/mL) and minimal syncytia formation of the producer cell during the method of production.

Provided herein is a lipid particle produced by any of the provided methods. Also provided herein is a lentiviral particle produce by any of the provided methods.

Provided herein is a lipid particle comprising any of the provided truncated BaEV envelope glycoproteins. Provided herein is a composition comprising a plurality of any of the provided lentiviral particles. Provided herein is a composition comprising a plurality of any of the provided lipid particles.

In some of any of the provided embodiments, the composition further comprises a pharmaceutically acceptable excipient.

Provided herein is a method of transducing a cell, the method comprising contacting a cell with any of the provided lentiviral particles or any of the provided compositions. In some of any of the provided embodiments, the lipid particle or lentiviral vector comprises an exogenous agent and the transduction introduces the exogenous agent into the cell.

Provided herein is a method of delivering an exogenous agent into a cell, the method comprising contacting any of the provided lentiviral particles or any of the provided lipid particles or any of the provided compositions with a cell.

In some of any of the provided embodiments, the contacting is in vitro or ex vivo. In some of any of the provided embodiments, the contacting is in vivo in a subject.

Provided herein is a method of delivering an exogenous agent to a cell in a subject, the method comprising administering to the subject any of the provided lentiviral particles, lipid particles, or compositions.

In some of any of the provided embodiments, the cell is a hematopoietic lineage cell. In some of any of the provided embodiments, the cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some of any of the provided embodiments, the cell is selected from monocytes, macrophages, neutrophils, basophils, eosinophils, erythrocytes, megakaryocytes and platelets. In some of any of the provided embodiments, the cell is selected from T cells, B cells, natural killer (NK) cells and innate lymphoid cells. In some of any of the provided embodiments, the cell is a hematopoietic stem cell (HSC). In some of any of the provided embodiments, the subject has received a hematopoietic stem cell transplant.

In some of any of the provided embodiments, the exogenous agent is a protein or a nucleic acid, optionally wherein the nucleic acid is a DNA or RNA. In some of any of the provided embodiments, the exogenous agent is or encodes a therapeutic agent for treating a disease or condition in the subject. In some of any of the provided embodiments, the exogenous agent is or encodes a membrane protein, optionally a chimeric antigen receptor, for targeting an antigen associated with a disease or condition in the subject. In some of any of the provided embodiments, the exogenous agent is for use in gene therapy to correct a genetic deficiency or replaces a deficient or missing gene in the subject. In some of any of the provided embodiments, the subject is a human subject.

### BRIEF DESCRIPTION OF THE DRAWINGS

FIG. 1 depicts viral titer of lentiviral vector particles produced from producer cells transfected with packaging plasmids and various truncated BaEV envelope glycoproteins containing inhibitory R peptides of different lengths.

FIG. 2 depicts syncytia formation of producer cells during production of lentiviral vector particles by transfection of the producer cells with packaging plasmids and various truncated BaEV envelope glycoproteins containing inhibitory R peptides of different lengths.

FIG. 3 depicts viral titer of an exemplary lentiviral vector preparation pseudotyped with a truncated BaEV envelope glycoprotein containing a partial inhibitory R peptide with 8 contiguous amino a terminal amino acids of the R peptide (R+8) of wild-type BaEV envelope protein, following production from producer cells transfected with packaging plasmids and different concentrations of a plasmid encoding the R+8 truncated BaEV envelope glycoprotein.

FIG. 4. depicts the percentage of GFP+ cells eight days after transduction with BaEV+8, BaEVTR, BaEVRless, or VSV-G pseudotyped vectors.

### DETAILED DESCRIPTION

Provided herein are lipid particles, such as lentiviral particles, pseudotyped with a truncated Baboon Endogenous Retrovirus (BaEV) envelope glycoprotein containing a cytoplasmic tail with a partial fusion inhibitory R peptide relative to a full length wild-type BaEV envelope glycoprotein. Also provided herein are lipid particles, such as lentiviral particles, pseudotyped with a truncated Baboon Endogenous Retrovirus (BaEV) envelope glycoprotein that comprise (i) a glycoprotein 70 (g70) subunit or a biologically active portion thereof, and (ii) a glycoprotein p20E (p20E) subunit comprising a partial fusion inhibitory R peptide. In particular embodiments, the R peptide contains at least three contiguous amino-terminal amino acids of the inhibitory R peptide. In particular embodiments, the partial fusion inhibitory R peptide is capable of mediating the fusion of the truncated BaEV envelope glycoprotein to a desired target cell. In some embodiments, the truncated BaEV envelope glycoprotein is embedded in the lipid particle. The lipid particle may be a non-viral particle, a viral particle or a viral-like particle (VLP).

The provided embodiments are based on the BaEV envelope glycoprotein which exhibits transduction properties particularly suitable for gene transfer into hematopoietic cells, including hematopoietic stem cells (HSC) and resting T and B cells. This provides for a delivery vector for delivery of a therapeutic genes to HSCs, able to differentiate into all hematopoietic lineages, in order to replace or correct genes in connection with gene therapy methods. Further, efficient gene transfer into quiescent or resting T and B lymphocytes for gene therapy or immunotherapy also has a number of advantages in connection with ex vivo and in vivo methods of viral particle delivery.

The BaEV envelope glycoproteins have structural and functional features in common with other retroviral envelope glycoproteins, including an ectodomain, a transmembrane domain and a cytoplasmic tail domain. BaEV envelope glycoproteins are synthesized as inactive precursors which are N-glycosylated and are processed by host cell furin or furin-like proteases in the Golgi into two subunits, the surface unit protein or gp70, and the transmembrane protein p20E. The cleavage site between the gp70 and the p20E requires the minimal sequence [KR]-X-[KR]-R (wherein X is any amino acid), and the gp70 and the p20E remain associated in a labile interaction that may include a disulfide bond. The gp70 attaches the virus to the host cell by binding a receptor, which triggers the refolding of the p20E, which is believed to promote fusion with the host cell membrane. The p20E acts as a class I viral fusion protein, which shares structural and functional features in common with fusion proteins of many families (e.g., HIV-1 gp41 or influenza virus hemagglutinin [HA]). Under native conditions, the BaEV envelope glycoprotein also undergoes a second cleavage in the cytoplasmic domain or cytoplasmic tail of the p20E. The C-terminal 17 amino acids of the cytoplasmic tail, the fusion inhibitory R peptide, contain a tyrosine endocytosis signal YXXL, which is removed as a result of proteolytic cleavage by the viral protease. Removal of the R peptide has been associated with promotion of fusogenicity of the envelope glycoprotein (Beneviste et al (1974) Nature 248:17-20; Todaro et al (1974) Cell 2:55-61; Aguilar et al. (2003) Journal of Virology 77(2): 1281-1291).

While the mechanism by which the R-peptide cleavage enhances fusogenicity is not well understood, the efficiency of fusion of BaEV envelope glycoproteins can be improved by engineering cleavage of the R peptide in the absence of a viral protease. Mutations in the BaEV that completely cleave the 17 amino acid R-peptide, have been identified (U.S. Pat. No. 9,249,426, Bernardin et al. (2019) Blood. 3(3):461-475). In addition, although there is low homology between R-peptides, including only 33% homology between the murine leukemia virus (MuLV) and gibbon ape leukemia virus (GaLV), R peptides are interchangeable (Christodoulopoulos et al (2001) J. Viral 75(4): 4129-4138). Several such studies in which the cytoplasmic tail of variant BaEV envelope glycoproteins has been replaced by the MuLV have been identified (U.S. Pat. No. 9,249,426, Bernardin et al. (2019) Blood. 3(3):461-475).

However, truncation of the full R peptide from the cytoplasmic tail renders the envelope glycoprotein highly fusogenic, resulting in massive syncytium formation (Olsen et al. (1999) Journal of Virology 8975-8981; Ragheb et al (1994) J Virol. 68:3220-3231; Rein et al (1994) J Virol 1773-1781). Various envelope glycoproteins, including those form MuLV and BaEV, display greater ability to induce syncytia in cell-cell fusion assays when lacking the R-peptide, compared to the full-length R peptide, across various cell lines (Aguilar (2003) J. Virol. 77(2): 1281-1291). In addition, syncytia formation during lentiviral vector production has been shown to result in low titer, even under conditions attempting to optimize higher titer protocols (Bauler et al. (2019) Molecular Therapy vol. 17; Noguchi et al. (2020) ASGCT 23^{rd} Annual Meeting. Poster=988).

While particles incorporating BaEVs lacking the wild type R peptide have been generated, it is found herein that hyperfusogenic mutants with low syncytium formation result from specific tail truncations of BaEV envelope glycoproteins. In addition, it is found herein that when expressing specific amino acid truncations of the R peptide, mutant BaEV envelope glycoproteins result in improved titer of a lentiviral preparation. For example, it is found that BaEV envelope glycoproteins that contain 8 amino acids of the R peptide result in 5 fold higher titer than BaEV envelope glycoproteins that express 10 amino acids of the R peptide.

The provided lipid particles, such as lentiviral particles, incorporate a truncated BaEV) envelope glycoproteins containing a cytoplasmic tail with a partial fusion inhibitory R peptide relative to the inhibitory R peptide of a full length wild-type BaEV envelope glycoprotein. The partial fusion inhibitory peptide can comprise one, two, three, four, five, six, seven, eight or nine contiguous amino-terminal amino acids of the inhibitory R peptide. In some embodiments, the provided lipid particles, such as lentiviral vectors, exhibit high titer following their production by producer cells, particular compared to full-length BaEV envelope glycoproteins containing a full inhibitory R peptide. Moreover, the provided lipid particles, such as lentiviral vectors, exhibit lower fusogenic activity resulting in low or minimal syncytia formation of the producer cells, particular compared to a mutant BaEV envelope glycoprotein that lacks the complete R peptide (R-less).

Also provided are particles comprising truncated Baboon Endogenous Retrovirus (BaEV) envelope glycoproteins containing a cytoplasmic tail with a partial fusion inhibitory R peptide relative to a wild-type BaEV envelope glycoprotein. Particles can include lipid particles, a viral particles, a viral-like particles (VLP), non-viral particles or synthetic particles, or any of the particles described herein.

Also provided are particles additionally containing one or more exogenous agents, such as for delivery of a diagnostic or therapeutic agent to cells, including following in vivo administration to a subject. In some embodiments, the exogenous agent may be a transgene, such as a nucleic acid encoding a desired protein, or may be heterologous protein. In some aspects, the transgene can be used in connection with gene therapy, such as to replace a gene in a cell that is deficient or defective for the gene. In some embodiments, the exogenous agent may encode or is a protein that is desired to be delivered to a target cell. In some embodiments, the exogenous agent encodes or is a chimeric antigen receptor (CAR).

In some embodiments, the exogenous agent is or encodes a factor associated with gene editing. In some embodiments, the exogenous agent is or encodes a factor associated with base editing and/or prime editing (i.e, target-primed reverse transcription (TPRT)). In some embodiments, the exogenous agent encodes or is a nuclease, such as for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the exogenous agent is or encodes a transposase and/or recombinase. In some embodiments, the exogenous agent is or encodes a DNA polymerase, RNA polymerase, or reverse-transcriptase.

Also provided herein are methods and uses of the truncated BaEV envelope glycoproteins and particles, such in diagnostic and therapeutic methods. Also provided are polynucleotides, methods for engineering, preparing, and producing the glycoproteins and particles, and kits and devices containing and for using, producing and administering the particles.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, “lipid particle” refers to any biological or synthetic particle that contains a bilayer of amphipathic lipids enclosing a lumen or cavity. Typically, a particle does not contain a nucleus. Examples of lipid particles include solid particles such as nanoparticles, viral-derived particles or cell-derived particles. Such lipid particles include, but are not limited to, viral-based particles such as virus-like particles or viral vectors (e.g., lentiviral vectors), exosomes, enucleated cells, various vesicles, such as a microvesicle, a membrane vesicle, an extracellular membrane vesicle, a plasma membrane vesicle, a giant plasma membrane vesicle, an apoptotic body, a mitoparticle, a pyrenocyte, or a lysosome. In some embodiments, a lipid particle can be a fusosome. In some embodiments, the lipid particle is not a platelet.

The term “viral-based particle” can be any type of lipid particle that is derived from a virus or from viral protein, for example viral vector particles and virus-like particles.

The terms “viral vector particle” and “viral vector” are used interchangeably herein. A viral vector particle can be any type of lipid particle which comprises one or more viral structural proteins in addition to at least one non-structural viral genomic component or functional fragment thereof (i.e., a polymerase, an integrase, a protease or other non-structural component).

The term “virus-like particle” or VLP can be any type of particle that features at least one viral structural protein and is devoid of viral genetic material.

As used herein, “fusosome” refers to a particle containing a bilayer of amphipathic lipids enclosing a lumen or cavity and a fusogen that interacts with the amphipathic lipid bilayer. In some embodiments, the fusosome comprises an exogenous agent. In some embodiments, the exogenous agent is a nucleic acid (e.g. DNA or RNA), a peptide or a protein. In some embodiments, the fusosome is a membrane enclosed preparation. In some embodiments, the fusosome is derived from a source cell.

As used herein, “fusosome composition” refers to a composition comprising one or more fusosomes.

As used herein, “fusogen” refers to an agent or molecule that creates an interaction between two membranes, including membrane enclosed lumens. In embodiments, the fusogen facilitates fusion of the membranes. In other embodiments, the fusogen creates a connection, e.g., a pore, between two membranes or lumens (e.g., a lumen of a retroviral vector and a cytoplasm of a target cell).

As used herein, a “retroviral nucleic acid” refers to a nucleic acid containing at least the minimal sequence requirements for packaging into a retrovirus or retroviral vector, alone or in combination with a helper cell, helper virus, or helper plasmid. In some embodiments, the retroviral nucleic acid comprises one or more of (e.g., all of) a 5′ LTR (e.g., to promote integration), U3 (e.g., to activate viral genomic RNA transcription), R (e.g., a Tat-binding region), U5, a 3′ LTR (e.g., to promote integration), a packaging site (e.g., psi (Y)), RRE (e.g., to bind to Rev and promote nuclear export). The retroviral nucleic acid can comprise RNA (e.g., when part of a virion) or DNA (e.g., when being introduced into a source cell or after reverse transcription in a recipient cell). In some embodiments, the retroviral nucleic acid is packaged using a helper cell, helper virus, or helper plasmid which comprises one or more of (e.g., all of) gag, pol, and env.

As used herein, “percent (%) amino acid sequence identity” and “homology” with respect to a peptide, polypeptide or antibody sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

An amino acid substitution may include but are not limited to the replacement of one amino acid in a polypeptide with another amino acid. Exemplary substitutions are shown in Table 1. Amino acid substitutions may be introduced into an antibody of interest and the products screened for a desired activity, for example, retained/improved binding.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g. fragment or species variant) can be determined by alignment to a reference sequence by structural alignment methods. By aligning the sequences, one skilled in the art can identify corresponding residues, for example, using conserved and identical amino acid residues as guides.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, for example, in the case of an RNA polynucleotide. Thus, a DNA polynucleotide that is contained in a vector inside a host cell may be referred to as “isolated”.

The term “effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

An “exogenous agent” as used herein with reference to a particle, refers to an agent that is neither comprised by nor encoded in the corresponding wild-type virus or fusogen made from a corresponding wild-type source cell. In some embodiments, the exogenous agent does not naturally exist, such as a protein or nucleic acid that has a sequence that is altered (e.g., by insertion, deletion, or substitution) relative to a naturally occurring protein. In some embodiments, the exogenous agent does not naturally exist in the source cell. In some embodiments, the exogenous agent exists naturally in the source cell but is exogenous to the virus. In some embodiments, the exogenous agent does not naturally exist in the recipient cell. In some embodiments, the exogenous agent exists naturally in the recipient cell, but is not present at a desired level or at a desired time. In some embodiments, the exogenous agent comprises RNA or protein.

As used herein, a “promoter” refers to a cis-regulatory DNA sequence that, when operably linked to a gene coding sequence, drives transcription of the gene. The promoter may comprise a transcription factor binding sites. In some embodiments, a promoter works in concert with one or more enhancers which are distal to the gene.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary and topical administration.

A “disease” or “disorder” as used herein refers to a condition where treatment is needed and/or desired.

As used herein, the terms “treat,” “treating,” or “treatment” refer to ameliorating a disease or disorder, e.g., slowing or arresting or reducing the development of the disease or disorder or reducing at least one of the clinical symptoms thereof. For purposes of this disclosure, ameliorating a disease or disorder can include obtaining a beneficial or desired clinical result that includes, but is not limited to, any one or more of: alleviation of one or more symptoms, diminishment of extent of disease, preventing or delaying spread (for example, metastasis, for example metastasis to the lung or to the lymph node) of disease, preventing or delaying recurrence of disease, delay or slowing of disease progression, amelioration of the disease state, inhibiting the disease or progression of the disease, inhibiting or slowing the disease or its progression, arresting its development, and remission (whether partial or total).

The terms “individual” and “subject” are used interchangeably herein to refer to an animal; for example a mammal. The term patient includes human and veterinary subjects. In some embodiments, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. The subject can be male or female and can be any suitable age, including infant, juvenile, adolescent, adult, and geriatric subjects. In some examples, an “individual” or “subject” refers to an individual or subject in need of treatment for a disease or disorder. In some embodiments, the subject to receive the treatment can be a patient, designating the fact that the subject has been identified as having a disorder of relevance to the treatment, or being at adequate risk of contracting the disorder. In particular embodiments, the subject is a human, such as a human patient.

### II. Truncated BaEV Fusogen

Provided herein are truncated Baboon Endogenous Retrovirus (BaEV) envelope glycoproteins that are or can be incorporated into a lipid particle, such as a viral particle, including a lentiviral particle or lentiviral-like particle. For instance, provided herein are lentiviral particles that are pseudotyped with any of the provided truncated BaEV envelope glycoproteins. Also provided herein are polynucleotides encoding the truncated BaEV envelope glycoproteins.

Wild-type BaEV envelope glycoproteins are retroviral envelope proteins containing a C-terminal cytoplasmic tail (e.g. corresponding to SEQ ID NO:4, or amino acids 512-545 of SEQ ID NO 24), a transmembrane domain (e.g. corresponding to SEQ ID NO: 3, or amino acids 489-511 of SEQ ID NO:24), and an extracellular domain (e.g corresponding to SEQ ID NO: 2, or amino acids 1-488 of SEQ ID NO:24). Maturation of the precursor protein in the Golgi, which requires the minimal sequence [KR]-X-[KR]-R (wherein X is any amino acid), results in two subunits, the surface unit protein or gp70, and the transmembrane protein p20E. The surface unit protein or gp70 (e.g. corresponding to SEQ ID NO: 25 or amino acids 1-358 of SEQ ID NO:24) and the transmembrane protein p20E (e.g. corresponding to SEQ ID NO:26, SEQ ID NO:27, or amino acids 359-545 of SEQ ID NO:24) remain associated in a labile interaction that may include a disulfide bond. In wild-type BaEV envelope glycoproteins, fusogenicity is controlled by a short, 17 amino acid sequence termed a fusion inhibitory R peptide (e.g. set forth in SEQ ID NO:22), which is localized on the C-terminal of the cytoplasmic tail domain. The fusion inhibitory R peptide harbors the tyrosine endocytosis signal YXXL, and its cleavage by the viral protease is thought to potentiate fusogenic activation through molecular rearrangements in the membrane-spanning domain and the extracellular region of the envelope glycoprotein (Salamango et al (2015) Journal of virology 89(24): 12492-12500).

In wild-type BaEV envelope glycoproteins, the gp70 mediates receptor binding to the ASCT-2 and ASCT-1 receptors on host cells. In some embodiments, the glycoprotein 70 (g70) subunit or a biologically active portion thereof binds the ASCT-2 and ASCT-1 receptors. In wild-type BaEV envelope glycoproteins, the p20E acts as a class I viral fusion protein. The interaction of the gp70 subunit with a host cell membrane triggers refolding of the p20E and is believed to activate the fusogenic potential by unmasking the fusion peptide.

In some embodiments, the truncated BaEV envelope glycoprotein comprise a cytoplasmic tail with a partial fusion inhibitory R peptide relative to a wild-type BaEV envelope glycoprotein, wherein the R peptide contains a contiguous portion of the inhibitory R peptide but lacks the full length R peptide of wild-type BaEV envelope glycoprotein. In some embodiments, the truncated BaEV envelope glycoprotein has a cytoplasmic tail that is composed of a partial inhibitory R peptide with at least one, at least two, or at least three contiguous amino-terminal amino acids of the inhibitory R peptide but less than the full-length R peptide relative to wild-type BaEV envelope glycoprotein. In some embodiments, the truncated BaEV envelope glycoprotein has a cytoplasmic tail that has a partial inhibitory R peptide composed of 1 to 16 contiguous amino-terminal amino acids of the inhibitory R peptide of the wild-type BaEV envelope glycoprotein, such as is composed of 1, 2, 3, 4, 5, 6, 7, 8, 9, 10, 11, 12, 12, 14, 15 or 16 amino-terminal amino acids of the inhibitory R peptide of the wild-type BaEV envelope glycoprotein.

In particular embodiments the partial fusion inhibitory R peptide comprises between 1 and 9 contiguous amino-terminal amino acids, but less than the full length, of the inhibitory R peptide of the wild-type BaEV envelope glycoprotein. In particular embodiments the partial fusion inhibitory R peptide comprises between 2 and 9 contiguous amino-terminal amino acids, but less than the full length, of the inhibitory R peptide of the wild-type BaEV envelope glycoprotein. In particular embodiments the partial fusion inhibitory R peptide comprises between 3 and 9 contiguous amino-terminal amino acids, but less than the full length, of the inhibitory R peptide of the wild-type BaEV envelope glycoprotein. In particular embodiments the partial fusion inhibitory R peptide comprises between 4 and 9 contiguous amino-terminal amino acids, but less than the full length, of the inhibitory R peptide of the wild-type BaEV envelope glycoprotein. In particular embodiments the partial fusion inhibitory R peptide comprises between 5 and 9 contiguous amino-terminal amino acids, but less than the full length, of the inhibitory R peptide of the wild-type BaEV envelope glycoprotein. In particular embodiments the partial fusion inhibitory R peptide comprises between 6 and 9 contiguous amino-terminal amino acids, but less than the full length, of the inhibitory R peptide of the wild-type BaEV envelope glycoprotein. In particular embodiments the partial fusion inhibitory R peptide comprises between 7 and 9 contiguous amino-terminal amino acids, but less than the full length, of the inhibitory R peptide of the wild-type BaEV envelope glycoprotein. In particular embodiments the partial fusion inhibitory R peptide comprises between 8 and 9 contiguous amino-terminal amino acids, but less than the full length, of the inhibitory R peptide of the wild-type BaEV envelope glycoprotein. In particular embodiments the partial fusion inhibitory R peptide comprises 9 contiguous amino-terminal amino acids, but less than the full length, of the inhibitory R peptide of the wild-type BaEV envelope glycoprotein.

In some embodiments, the truncated BaEV envelope glycoprotein contains a cytoplasmic tail that is less than the full length cytoplasmic tail of truncated BaEV envelope glycoprotein due to containing a partial inhibitory R peptide. In some embodiments, the truncated BaEV envelope glycoprotein contains a portion of the full-length cytoplasmic tail set forth in SEQ ID NO: 4. In some embodiment, the truncated BaEV envelope glycoprotein contains a cytoplasmic tail that contains between 18 and 33 contiguous amino-terminal amino acids of SEQ ID NO:4, such as 18, 19, 20, 21, 22, 23, 24, 25, 26, 27, 28, 29, 30, 31, 32 or 33 contiguous amino-terminal amino acids of SEQ ID NO:4. In some embodiments, the cytoplasmic tail of the truncated BaEV envelope is between 18 and 33 amino acids in length, such as is 18, 19, 20, 21, 22, 23, 24, 25, 26, 27, 28, 29, 30, 31, 32 or 33 amino acids in length.

In particular embodiments, the truncated BaEV envelope glycoprotein comprises a portion of the sequence set forth in any of SEQ ID NOs: 23 or 24 that is a functionally active variant or biologically active portion thereof that retains fusogenic activity. In some embodiments, the truncated BaEV envelope glycoprotein lacks up to 16 contiguous amino acids from the C-terminal cytoplasmic tail of SEQ ID NO: 23 or SEQ ID NO:24. In some embodiments, the truncated BaEV envelope glycoprotein lacks from 1 to 16 contiguous amino acids of the C-terminal cytoplasmic tail of SEQ ID NO: 23 or SEQ ID NO:24, such as 1, 2, 3, 4, 5, 6, 7, 8, 9, 10, 11, 12, 13, 14, 15 or 16 contiguous amino acids of the C-terminal cytoplasmic tail of SEQ ID NO: 23 or SEQ ID NO:24. In particular embodiments, the truncated BaEV envelope glycoprotein lacks from 8 to 14 contiguous amino acids of the C-terminal cytoplasmic tail of SEQ ID NO: 23 or SEQ ID NO:24. In some embodiments, the truncated BaEV envelope glycoprotein lacks from 8 to 13 contiguous amino acids of the C-terminal cytoplasmic tail of SEQ ID NO: 23 or SEQ ID NO:24.

In some embodiments, the provided truncated Baboon Endogenous Retrovirus (BaEV) envelope glycoproteins, such as when incorporated into a lipid particle (e.g. lentiviral particle), are composed of two chains that include (i) a glycoprotein 70 (g70) subunit or a biologically active portion thereof, and (ii) a portion of the glycoprotein p20E (p20E) subunit containing the cytoplasmic domain with the partial fusion inhibitory R peptide as described.

In particular embodiments, the truncated BaEV envelope glycoprotein gp70 subunit has the sequence set forth in SEQ ID NO:25, or is a functionally or biologically active variant thereof that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25.

In particular embodiments, the portion of the truncated BaEV envelope glycoprotein p20E subunit has the sequence set forth in SEQ ID NO: 26, or is a functionally or biologically active variant thereof that retains fusogenic activity, and comprises the partial inhibitory R peptide. In some embodiments, the functionally or biologically active variant thereof of the p20E subunit comprises a sequence that exhibits at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide. In some embodiments, the portion of the p20E subunit has the sequence set forth in any of SEQ ID NOS: 44-60.

In particular embodiments, the provided truncated BaEV envelope glycoproteins, such as when incorporated into a lipid particle (e.g. lentiviral particle), exhibit fusogenic activity, which is mediated by the partial fusion inhibitory R peptide. In some embodiments, the provided truncated BaEV envelope glycoproteins containing a partial fusion inhibitory R peptide retain fusogenic activity of wild-type BaEV envelope glycoprotein. Fusogenic activity includes the ability of the truncated BaEV envelope glycoprotein to promote or facilitate fusion of two membrane lumens, and a cytoplasm of a target cell, e.g. a cell that contains a surface receptor or molecule that is recognized or bound by the truncated BaEV envelope protein. In some embodiments, the truncated BaEV envelope glycoproteins provided herein bind the neutral amino acid transporter receptors ASCT-2 or ASCT-1. The ASCT-2 and ASCT-1 receptors share approximately 57% sequence identity, are differentially expressed in cells (e.g. T and B cells) and are transporters of an overlapping but non-identical set of neutral amino acids (Colmartino et al. (2019) Frontiers in Immunology 10(2873): 1-7; Girard-Gagnepain et al (2014) Blood 124:1221-1231; Levy et al. (2016) Journal of Thrombosis and Haemostasis 14:2478-2492).

Reference to retaining fusogenic activity includes activity that is between 10% and at or about 150% or more of the level or degree of binding of the corresponding wild-type BaEV envelope glycoprotein, such as set forth in SEQ ID NO:24. In some embodiments, the fusogenic activity of a truncated BaEV envelope glycoprotein is between 50% and 125% of the level or degree of binding of the corresponding wild-type BaEV envelope glycoprotein, such as set forth in SEQ ID NO:24. In some embodiments, the fusogenic activity of a truncated BaEV envelope glycoprotein is between 80% and 120% of the level or degree of binding of the corresponding wild-type BaEV envelope glycoprotein, such as set forth in SEQ ID NO:24.

In some embodiments, the fusogenic activity of a truncated BaEV envelope glycoprotein is less than the fusogenic activity of a BaEV envelope glycoprotein that lacks the full inhibitory R peptide (R-Less). For instance, in some embodiments, the fusogenic activity of a truncated BaEV envelope glycoprotein is less than the fusogenic activity of a R-Less BaEV envelope glycorprotein that has a 17 amino acid truncation in the distal C-terminal portion of wild-type BaEV envelope glycoprotein, corresponding to amino acids 529-545, of SEQ ID NO:24. In some embodiments, an R-Less BaEV envelope glycoprotein has the sequence of amino acids set forth in SEQ ID NO:28. In some embodiments, a provided truncated BaEV envelope glycoprotein exhibits 10% to 90% of the fusogenic activity of a R-Less BaEV envelope glycoprotein, such as less than at or about 90%, at or about 85%, at or about 80%, at or about 75%, at or about 70%, at or about 60%, at or about 50%, at or about 40%, at or about 30%, at or about 20% or at or about 10% or less of the fusogenic activity of an R-Less BaEV envelope glycoprotein, e.g. as set forth in SEQ ID NO:28. In some embodiments, the reduced fusogenic activity avoids high levels of cell fusion during production of provided lipid particles, e.g. lentiviral particles. For instance, incorporation of a truncated BaEV envelope glycoprotein during methods of producing a lipid particle, e.g. lentiviral particle, avoids or reduces high levels of syncytia formation of producer cells during production of the particles.

In particular embodiments, the truncated BaEV envelope glycoprotein contains a partial fusion inhibitory R peptide that contains nine contiguous amino terminal amino acids of the wild type inhibitory R peptide (e.g. R+9). In particular embodiments, the truncated BaEV envelope glycoprotein contains a partial fusion inhibitory R peptide that is set forth as amino acids 1 to 9 of SEQ ID NO:22. In some embodiment, the BaEV envelope glycoprotein has a cytoplasmic tail set forth in SEQ ID NO:14. In some embodiments, the truncated BaEV envelope glycoprotein has a sequence that exhibits at least 85%, at least 90% or at least 95% sequence identity to the sequence set forth in SEQ ID NO:37 and that contains a cytoplasmic domain with a partial inhibitor peptide of nine amino acids, e.g. that is set forth as amino acids 1 to 9 of SEQ ID NO:22. In some embodiments, the truncated BaEV envelope glycoprotein has the sequence set forth in SEQ ID NO:37. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:53. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains eight contiguous amino terminal amino acids of the wild type inhibitory R peptide (e.g. R+8). In particular embodiments, the truncated BaEV envelope glycoprotein contains a partial fusion inhibitory R peptide that is set forth as amino acids 1 to 8 of SEQ ID NO:22. In some embodiment, the BaEV envelope glycoprotein has a cytoplasmic tail set forth in SEQ ID NO: 13. In some embodiments, the truncated BaEV envelope glycoprotein has a sequence that exhibits at least 85%, at least 90% or at least 95% sequence identity to the sequence set forth in SEQ ID NO:36 and that contains a cytoplasmic domain with a partial inhibitor peptide of eight amino acids, e.g. that is set forth as amino acids 1 to 8 of SEQ ID NO:22. In some embodiments, the truncated BaEV envelope glycoprotein has the sequence set forth in SEQ ID NO:36. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:52. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains seven contiguous amino terminal amino acids of the wild type inhibitory R peptide (e.g. R+7). In particular embodiments, the truncated BaEV envelope glycoprotein contains a partial fusion inhibitory R peptide that is set forth as amino acids 1 to 7 of SEQ ID NO:22. In some embodiment, the BaEV envelope glycoprotein has a cytoplasmic tail set forth in SEQ ID NO:12. In some embodiments, the truncated BaEV envelope glycoprotein has a sequence that exhibits at least 85%, at least 90% or at least 95% sequence identity to the sequence set forth in SEQ ID NO:35 and that contains a cytoplasmic domain with a partial inhibitor peptide of seven amino acids, e.g. that is set forth as amino acids 1 to 7 of SEQ ID NO:22. In some embodiments, the truncated BaEV envelope glycoprotein has the sequence set forth in SEQ ID NO:35. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:51. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains six contiguous amino terminal amino acids of the wild type inhibitory R peptide (e.g. R+6). In particular embodiments, the truncated BaEV envelope glycoprotein contains a partial fusion inhibitory R peptide that is set forth as amino acids 1 to 6 of SEQ ID NO:22. In some embodiment, the BaEV envelope glycoprotein has a cytoplasmic tail set forth in SEQ ID NO: 11. In some embodiments, the truncated BaEV envelope glycoprotein has a sequence that exhibits at least 85%, at least 90% or at least 95% sequence identity to the sequence set forth in SEQ ID NO:34 and that contains a cytoplasmic domain with a partial inhibitor peptide of six amino acids, e.g. that is set forth as amino acids 1 to 6 of SEQ ID NO:22. In some embodiments, the truncated BaEV envelope glycoprotein has the sequence set forth in SEQ ID NO:34. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:50. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains five contiguous amino terminal amino acids of the wild type inhibitory R peptide (e.g. R+5). In particular embodiments, the truncated BaEV envelope glycoprotein contains a partial fusion inhibitory R peptide that is set forth as amino acids 1 to 5 of SEQ ID NO:22. In some embodiment, the BaEV envelope glycoprotein has a cytoplasmic tail set forth in SEQ ID NO:10. In some embodiments, the truncated BaEV envelope glycoprotein has a sequence that exhibits at least 85%, at least 90% or at least 95% sequence identity to the sequence set forth in SEQ ID NO:33 and that contains a cytoplasmic domain with a partial inhibitor peptide of five amino acids, e.g. that is set forth as amino acids 1 to 5 of SEQ ID NO:22. In some embodiments, the truncated BaEV envelope glycoprotein has the sequence set forth in SEQ ID NO:33. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:49. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains four contiguous amino terminal amino acids of the wild type inhibitory R peptide (e.g. R+4). In particular embodiments, the truncated BaEV envelope glycoprotein contains a partial fusion inhibitory R peptide that is set forth as amino acids 1 to 4 of SEQ ID NO:22. In some embodiment, the BaEV envelope glycoprotein has a cytoplasmic tail set forth in SEQ ID NO:9. In some embodiments, the truncated BaEV envelope glycoprotein has a sequence that exhibits at least 85%, at least 90% or at least 95% sequence identity to the sequence set forth in SEQ ID NO:32 and that contains a cytoplasmic domain with a partial inhibitor peptide of four amino acids, e.g. that is set forth as amino acids 1 to 4 of SEQ ID NO:22. In some embodiments, the truncated BaEV envelope glycoprotein has the sequence set forth in SEQ ID NO:32. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:48. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains three contiguous amino terminal amino acids of the wild type inhibitory R peptide (e.g. R+3). In particular embodiments, the truncated BaEV envelope glycoprotein contains a partial fusion inhibitory R peptide that is set forth as amino acids 1 to 3 of SEQ ID NO:22. In some embodiment, the BaEV envelope glycoprotein has a cytoplasmic tail set forth in SEQ ID NO:8. In some embodiments, the truncated BaEV envelope glycoprotein has a sequence that exhibits at least 85%, at least 90% or at least 95% sequence identity to the sequence set forth in SEQ ID NO:31 and that contains a cytoplasmic domain with a partial inhibitor peptide of three amino acids, e.g. that is set forth as amino acids 1 to 3 of SEQ ID NO:22. In some embodiments, the truncated BaEV envelope glycoprotein has the sequence set forth in SEQ ID NO:31. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:47. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains two contiguous amino terminal amino acids of the wild type inhibitory R peptide (e.g. R+2). In particular embodiments, the truncated BaEV envelope glycoprotein contains a partial fusion inhibitory R peptide that is set forth as amino acids 1 to 2 of SEQ ID NO:22. In some embodiment, the BaEV envelope glycoprotein has a cytoplasmic tail set forth in SEQ ID NO:7. In some embodiments, the truncated BaEV envelope glycoprotein has a sequence that exhibits at least 85%, at least 90% or at least 95% sequence identity to the sequence set forth in SEQ ID NO:30 and that contains a cytoplasmic domain with a partial inhibitor peptide of two amino acids, e.g. that is set forth as amino acids 1 to 1 of SEQ ID NO:22. In some embodiments, the truncated BaEV envelope glycoprotein has the sequence set forth in SEQ ID NO:30. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:46. In some embodiments, the gp70 subunit is s and the portion of the p20E are associated via an inter-subunit disulfide bond.

In particular embodiments, the truncated BaEV envelope glycoprotein contains a partial fusion inhibitory R peptide that contains one contiguous amino terminal amino acids of the wild type inhibitory R peptide (e.g. R+1). In particular embodiments, the truncated BaEV envelope glycoprotein contains a partial fusion inhibitory R peptide that is set forth as amino acid 1 of SEQ ID NO:22. In some embodiment, the BaEV envelope glycoprotein has a cytoplasmic tail set forth in SEQ ID NO:6. In some embodiments, the truncated BaEV envelope glycoprotein has a sequence that exhibits at least 85%, at least 90% or at least 95% sequence identity to the sequence set forth in SEQ ID NO:29 and that contains a cytoplasmic domain with a partial inhibitory peptide of one amino acid, e.g. that is set forth as amino acid of SEQ ID NO:22. In some embodiments, the truncated BaEV envelope glycoprotein has the sequence set forth in SEQ ID NO:29. In some embodiments, the truncated BaEV envelope glycoprotein is a two chain form containing the gp70 or a biologically active portion thereof (e.g. set forth in SEQ ID NO:25) and the portion of the p20E subunit containing the cytoplasmic domain with the partial inhibitory polypeptide. In some embodiments, the truncated BaEV envelope glycoprotein is a two chain form containing the gp70 subunit set forth in SEQ ID NO:25 and the portion of the p20E subunit set forth in SEQ ID NO:45. In some embodiments, the gp70 subunit and the portion of the p20E are associated via an inter-subunit disulfide bond.

### A. Polynucleotides

Provided herein are polynucleotides comprising a nucleic acid sequence encoding a truncated BaEV envelope glycoprotein containing a cytoplasmic tail with a partial fusion inhibitory R peptide relative to a wild-type BaEV envelope glycoprotein described herein. The polynucleotides may include a sequence of nucleotides encoding any of the truncated BaEV envelope glycoproteins described above.

In some embodiments, the polynucleotide encodes a truncated BaEV envelope glycoprotein containing a cytoplasmic tail with a partial fusion inhibitory R peptide that contains nine contiguous amino terminal amino acids of the wild type inhibitory R peptide (e.g. R+9). In some embodiments, the polynucleotide encodes a truncated BaEV envelope glycoprotein that has a sequence of amino acids that exhibits at least 85%, at least 90% or at least 95% sequence identity to SEQ ID NO:37 and that contains a cytoplasmic domain with a partial inhibitory peptide of nine amino acid, e.g. that is set forth as amino acid 1-9 of SEQ ID NO:22. In some embodiments, the polynucleotide encodes a truncated BaEV envelope glycoprotein set forth in SEQ ID NO:37.

In some embodiments, the polynucleotide encodes a truncated BaEV envelope glycoprotein containing a cytoplasmic tail with a partial fusion inhibitory R peptide that contains eight contiguous amino terminal amino acids of the wild type inhibitory R peptide (e.g. R+8). In some embodiments, the polynucleotide encodes a truncated BaEV envelope glycoprotein that has a sequence of amino acids that exhibits at least 85%, at least 90% or at least 95% sequence identity to SEQ ID NO:36 and that contains a cytoplasmic domain with a partial inhibitory peptide of eight amino acid, e.g. that is set forth as amino acid 1-8 of SEQ ID NO:22. In some embodiments, the polynucleotide encodes a truncated BaEV envelope glycoprotein set forth in SEQ ID NO:36.

In some embodiments, the polynucleotide encodes a truncated BaEV envelope glycoprotein containing a cytoplasmic tail with a partial fusion inhibitory R peptide that contains seven contiguous amino terminal amino acids of the wild type inhibitory R peptide (e.g. R+7). In some embodiments, the polynucleotide encodes a truncated BaEV envelope glycoprotein that has a sequence of amino acids that exhibits at least 85%, at least 90% or at least 95% sequence identity to SEQ ID NO:35 and that contains a cytoplasmic domain with a partial inhibitory peptide of seven amino acid, e.g. that is set forth as amino acid 1-7 of SEQ ID NO:22. In some embodiments, the polynucleotide encodes a truncated BaEV envelope glycoprotein set forth in SEQ ID NO:35.

In some embodiments, the polynucleotide encodes a truncated BaEV envelope glycoprotein containing a cytoplasmic tail with a partial fusion inhibitory R peptide that contains six contiguous amino terminal amino acids of the wild type inhibitory R peptide (e.g. R+6). In some embodiments, the polynucleotide encodes a truncated BaEV envelope glycoprotein that has a sequence of amino acids that exhibits at least 85%, at least 90% or at least 95% sequence identity to SEQ ID NO:34 and that contains a cytoplasmic domain with a partial inhibitory peptide of six amino acid, e.g. that is set forth as amino acid 1-6 of SEQ ID NO:22. In some embodiments, the polynucleotide encodes a truncated BaEV envelope glycoprotein set forth in SEQ ID NO:34.

In some embodiments, the polynucleotide encodes a truncated BaEV envelope glycoprotein containing a cytoplasmic tail with a partial fusion inhibitory R peptide that contains five contiguous amino terminal amino acids of the wild type inhibitory R peptide (e.g. R+5). In some embodiments, the polynucleotide encodes a truncated BaEV envelope glycoprotein that has a sequence of amino acids that exhibits at least 85%, at least 90% or at least 95% sequence identity to SEQ ID NO:33 and that contains a cytoplasmic domain with a partial inhibitory peptide of five amino acid, e.g. that is set forth as amino acid 1-5 of SEQ ID NO:22. In some embodiments, the polynucleotide encodes a truncated BaEV envelope glycoprotein set forth in SEQ ID NO:33.

In some embodiments, the polynucleotide encodes a truncated BaEV envelope glycoprotein containing a cytoplasmic tail with a partial fusion inhibitory R peptide that contains four contiguous amino terminal amino acids of the wild type inhibitory R peptide (e.g. R+4). In some embodiments, the polynucleotide encodes a truncated BaEV envelope glycoprotein that has a sequence of amino acids that exhibits at least 85%, at least 90% or at least 95% sequence identity to SEQ ID NO:32 and that contains a cytoplasmic domain with a partial inhibitory peptide of four amino acid, e.g. that is set forth as amino acid 1-4 of SEQ ID NO:22. In some embodiments, the polynucleotide encodes a truncated BaEV envelope glycoprotein set forth in SEQ ID NO:32.

In some embodiments, the polynucleotide encodes a truncated BaEV envelope glycoprotein containing a cytoplasmic tail with a partial fusion inhibitory R peptide that contains three contiguous amino terminal amino acids of the wild type inhibitory R peptide (e.g. R+3). In some embodiments, the polynucleotide encodes a truncated BaEV envelope glycoprotein that has a sequence of amino acids that exhibits at least 85%, at least 90% or at least 95% sequence identity to SEQ ID NO:31 and that contains a cytoplasmic domain with a partial inhibitory peptide of three amino acid, e.g. that is set forth as amino acid 1-3 of SEQ ID NO:22. In some embodiments, the polynucleotide encodes a truncated BaEV envelope glycoprotein set forth in SEQ ID NO:31.

In some embodiments, the polynucleotide encodes a truncated BaEV envelope glycoprotein containing a cytoplasmic tail with a partial fusion inhibitory R peptide that contains two contiguous amino terminal amino acids of the wild type inhibitory R peptide (e.g. R+2). In some embodiments, the polynucleotide encodes a truncated BaEV envelope glycoprotein that has a sequence of amino acids that exhibits at least 85%, at least 90% or at least 95% sequence identity to SEQ ID NO:30 and that contains a cytoplasmic domain with a partial inhibitory peptide of two amino acid, e.g. that is set forth as amino acid 1-2 of SEQ ID NO:22. In some embodiments, the polynucleotide encodes a truncated BaEV envelope glycoprotein set forth in SEQ ID NO:30.

In some embodiments, the polynucleotide encodes a truncated BaEV envelope glycoprotein containing a cytoplasmic tail with a partial fusion inhibitory R peptide that contains one contiguous amino terminal amino acids of the wild type inhibitory R peptide (e.g. R+1). In some embodiments, the polynucleotide encodes a truncated BaEV envelope glycoprotein that has a sequence of amino acids that exhibits at least 85%, at least 90% or at least 95% sequence identity to SEQ ID NO:29 and that contains a cytoplasmic domain with a partial inhibitory peptide of one amino acid, e.g. that is set forth as amino acid one of SEQ ID NO:22. In some embodiments, the polynucleotide encodes a truncated BaEV envelope glycoprotein set forth in SEQ ID NO:29.

In some embodiments, the polynucleotide can be a synthetic nucleic acid. Also provided are expression vectors containing any of the provided polynucleotides.

In some of any embodiments, expression of natural or synthetic nucleic acids is typically achieved by operably linking a nucleic acid encoding the gene of interest to a promoter and incorporating the construct into an expression vector. In some embodiments, vectors can be suitable for replication and integration in eukaryotes. In some embodiments, cloning vectors contain transcription and translation terminators, initiation sequences, and promoters useful for expression of the desired nucleic acid sequence. In some of any embodiments, a plasmid comprises a promoter suitable for expression in a cell.

In some embodiments, the polynucleotides contain at least one promoter that is operatively linked to control expression of the truncated BaEV envelope glycoprotein. For expression of the truncated BaEV envelope glycoprotein, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

In some embodiments, additional promoter elements, e.g., enhancers, regulate the frequency of transcriptional initiation. In some embodiments, additional promoter elements are located in the region 30-110 bp upstream of the start site, although a number of promoters have recently been shown to contain functional elements downstream of the start site as well. In some embodiments, spacing between promoter elements frequently is flexible, so that promoter function is preserved when elements are inverted or moved relative to one another. In some embodiments, the thymidine kinase (tk) promoter, the spacing between promoter elements can be increased to 50 bp apart before activity begins to decline. In some embodiments, depending on the promoter, individual elements can function either cooperatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR, in connection with the compositions disclosed herein (U.S. Pat. Nos. 4,683,202 and 5,928,906).

In some embodiments, a suitable promoter is the immediate early cytomegalovirus (CMV) promoter sequence. In some embodiments, the promoter sequence is a strong constitutive promoter sequence capable of driving high levels of expression of any polynucleotide sequence operatively linked thereto. In some embodiments, a suitable promoter is Elongation Growth Factor-1a (EF-1 a). In some embodiments, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

In some embodiments, the promoter is an inducible promoter. In some embodiments, the inducible promoter provides a molecular switch capable of turning on expression of the polynucleotide sequence which it is operatively linked when such expression is desired, or turning off the expression when expression is not desired. In some embodiments, inducible promoters comprise metallothionine promoter, a glucocorticoid promoter, a progesterone promoter, and a tetracycline promoter.

In some embodiments, exogenously controlled inducible promoters can be used to regulate expression of the truncated BaEV envelope glycoprotein. For example, radiation-inducible promoters, heat-inducible promoters, and/or drug-inducible promoters can be used to selectively drive transgene expression in, for example, targeted regions. In such embodiments, the location, duration, and level of transgene expression can be regulated by the administration of the exogenous source of induction.

In some embodiments, expression of the truncated BaEV envelope glycoprotein is regulated using a drug-inducible promoter. For example, in some cases, the promoter, enhancer, or transactivator comprises a Lac operator sequence, a tetracycline operator sequence, a galactose operator sequence, a doxycycline operator sequence, a rapamycin operator sequence, a tamoxifen operator sequence, or a hormone-responsive operator sequence, or an analog thereof. In some instances, the inducible promoter comprises a tetracycline response element (TRE). In some embodiments, the inducible promoter comprises an estrogen response element (ERE), which can activate gene expression in the presence of tamoxifen. In some instances, a drug-inducible element, such as a TRE, can be combined with a selected promoter to enhance transcription in the presence of drug, such as doxycycline. In some embodiments, the drug-inducible promoter is a small molecule-inducible promoter.

Any of the provided polynucleotides can be modified to remove CpG motifs and/or to optimize codons for translation in a particular species, such as human, canine, feline, equine, ovine, bovine, etc. species. In some embodiments, the polynucleotides are optimized for human codon usage (i.e., human codon-optimized). In some embodiments, the polynucleotides are modified to remove CpG motifs. In other embodiments, the provided polynucleotides are modified to remove CpG motifs and are codon-optimized, such as human codon-optimized. Methods of codon optimization and CpG motif detection and modification are well-known. Typically, polynucleotide optimization enhances transgene expression, increases transgene stability and preserves the amino acid sequence of the encoded polypeptide.

In order to assess the expression of the truncated BaEV envelope glycoprotein, the expression vector to be introduced into a cell can also contain either a selectable marker gene or a reporter gene or both to facilitate identification and selection of expressing particles, e.g. viral particles. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000, FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of the desired polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

### III. Lipid Particles and Methods for Generating Lipid Particles

Provided herein is a particle comprising a lipid bilayer, a lumen surrounded by the lipid bilayer and a truncated BaEV envelope protein, such as any as described, in which the truncated BaEV envelope protein is embedded within the lipid bilayer. In some embodiments, the provided lipid particles preferentially target hematopoietic cells (e.g. T cells), which is mediated by the tropism of the truncated BaEV envelope protein. In some embodiments, the lipid particle may additionally contain an exogenous agent (e.g. therapeutic agent) for delivery to a cell. In some embodiments, a lipid particle is introduced to a cell in the subject. Also provided are methods of delivering any of the provided lipid particles to a cell.

In some embodiments, the provided lipid particles exhibit fusogenic activity, which is mediated by the truncated BaEV envelope protein that facilitates merger or fusion of the two lumens of the lipid particle and the target cell membranes. Thus, among provided lipid particles are fusosomes. In some embodiments, the fusosome comprises a naturally derived bilayer of amphipathic lipids with the truncated BaEV envelope protein as a fusogen. In some embodiments, the fusosome comprises (a) a lipid bilayer, (b) a lumen (e.g., comprising cytosol) surrounded by the lipid bilayer; and (c) a fusogen that is exogenous or overexpressed relative to the source cell. In some embodiments, the truncated BaEV envelope protein disposed in the lipid bilayer. In some embodiments, the fusosome comprises several different types of lipids, e.g., amphipathic lipids, such as phospholipids. In some embodiments, the fusosome comprises a lipid bilayer as the outermost surface.

In some embodiments, the lipid particle includes a naturally derived bilayer of amphipathic lipids that encloses lumen or cavity. In some embodiments, the lipid particle comprises a lipid bilayer as the outermost surface. In some embodiments, the lipid bilayer encloses a lumen. In some embodiments, the lumen is aqueous. In some embodiments, the lumen is in contact with the hydrophilic head groups on the interior of the lipid bilayer. In some embodiments, the lumen is a cytosol. In some embodiments, the cytosol contains cellular components present in a source cell. In some embodiments, the cytosol does not contain components present in a source cell. In some embodiments, the lumen is a cavity. In some embodiments, the cavity contains an aqueous environment. In some embodiments, the cavity does not contain an aqueous environment.

In some embodiments, the lipid particle can be a viral particle, a virus-like particle, a nanoparticle, a vesicle, an exosome, a dendrimer, a lentivirus, a viral vector, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a lentiviral vector, a viral based particle, a virus like particle (VLP) or a cell based particle.

In some aspects, the lipid bilayer is derived from a source cell during a process to produce a lipid-containing particle. Exemplary methods for producing lipid-containing particles are described herein. In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In particular embodiments, the lipid particle is virally derived. In some embodiments, the lipid particle can be a viral-based particle, such as a viral vector particle (e.g. lentiviral vector particle) or a virus-like particle (e.g. a lentiviral-like particle). In some embodiments, the lipid bilayer is a viral envelope. In some embodiments, the viral envelope is obtained from a host cell. In some embodiments, the viral envelope is obtained by the viral capsid from the source cell plasma membrane. In some embodiments, the lipid bilayer is obtained from a membrane other than the plasma membrane of a host cell. In some embodiments, the viral envelope lipid bilayer is embedded with viral proteins, including viral glycoproteins.

In particular embodiments, the lipid particle is not virally derived. In some embodiments, the lipid particle can be a nanoparticle, a vesicle, an exosome, a dendrimer, an enucleated cell, a microvesicle, a membrane vesicle, an extracellular membrane vesicle, a plasma membrane vesicle, a giant plasma membrane vesicle, an apoptotic body, a mitoparticle, a pyrenocyte, a lysosome, another membrane enclosed vesicle, or a cell derived particle.

In some embodiments, the lipid bilayer includes membrane components of the host cell from which the lipid bilayer is derived, e.g., phospholipids, membrane proteins, etc. In some embodiments, the lipid bilayer includes a cytosol that includes components found in the cell from which the vehicle is derived, e.g., solutes, proteins, nucleic acids, etc., but not all of the components of a cell, e.g., lacking a nucleus. In some embodiments, the lipid bilayer is considered to be exosome-like. The lipid bilayer may vary in size, and in some instances have a diameter ranging from 30 and 300 nm, such as from 30 and 150 nm, and including from 40 to 100 nm.

In other aspects, the lipid bilayer includes synthetic lipid complex. In some embodiments, the synthetic lipid complex is a liposome. In some embodiments, the lipid bilayer is a vesicular structure characterized by a phospholipid bilayer membrane and an inner aqueous medium. In some embodiments, the lipid bilayer has multiple lipid layers separated by aqueous medium. In some embodiments, the lipid bilayer forms spontaneously when phospholipids are suspended in an excess of aqueous solution. In some examples, the lipid components undergo self-rearrangement before the formation of closed structures and entrap water and dissolved solutes between the lipid bilayers.

In some embodiments, the lipid particle comprises several different types of lipids. In some embodiments, the lipids are amphipathic lipids. In some embodiments, the amphipathic lipids are phospholipids. In some embodiments, the phospholipids comprise phosphatidylcholine, phosphatidylethanolamine, phosphatidylinositol, and phosphatidylserine. In some embodiments, the lipids comprise phospholipids such as phosphocholines and phosphoinositols. In some embodiments, the lipids comprise DMPC, DOPC, and DSPC.

In particular embodiments, an exogenous agent, such as a polynucleotide or polypeptide, is encapsulated within the lumen of a lipid particle. Embodiments of provided lipid particles may have various properties that facilitate delivery of a payload, such as, e.g., a desired transgene or exogenous agent, to a target cell. The exogenous agent may be a polynucleotide or a polypeptide. In some embodiments, a lipid particle provided herein is administered to a subject, e.g., a mammal, e.g., a human. In such embodiments, the subject may be at risk of, may have a symptom of, or may be diagnosed with or identified as having, a particular disease or condition. In one embodiment, the subject has cancer. In one embodiment, the subject has an infectious disease. In some embodiments, the lipid particle contains nucleic acid sequences (polynucleotide) encoding an exogenous agent or a polypeptide exogenous agent for treating the disease or condition.

The lipid particles can include spherical particles or can include particles of elongated or irregular shape.

In some embodiments, a composition of particles can be assessed for one or more features related to their size, including diameter, range of variation thereof above and below an average (mean) or median value of the diameter, coefficient of variation, polydispersity index or other measure of size of particles in a composition. Various methods for particle characterization can be used, including, but not limited to, laser diffraction, dynamic light scattering (DLS; also known as photon correlation spectroscopy) or image analysis, such as microscopy or automated image analysis.

In some embodiments, the provided lipid particle has a diameter of, or the average (mean) diameter of particles in a composition is, less than about 3 μm, less than about 2 μm, less than about 1 μm, less than about 900 nm, less than about 800 nm, less than about 700 nm, less than about 600 nm, less than about 500 m, less than about 400 nm, less than about 300, less than about 200 nm, less than about 150 nm, less than about 100 nm, less than about 50 nm, or less than about 20 nm. In some embodiments, the lipid particle has a diameter of, or the average (mean) diameter of particles in a composition is, less than about 400 nm. In another embodiment, the lipid particle has a diameter of, or the average (mean) diameter of particles in a composition is, less than about 150 nm. In some embodiments, the lipid particle has a diameter of, or the average (mean) diameter of particles in a composition is, between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments the median particle diameter in a composition of particles is between at or about 10 nm and at or about 1000 nM, between at or about 25 nm and at or about 500 nm, between at or about 40 nm and at or about 300 nm, between at or about 50 nm and at or about 250 nm, between at or about 60 nm and at or about 225 nm, between at or about 70 nm and at or about 200 nm, between at or about 80 nm and at or about 175 nm, or between at or about 90 nm and at or about 150 nm.

In some embodiments, 90% of the lipid particles in a composition fall within 50% of the median diameter of the lipid particles. In some embodiments, 90% of the lipid particles in a composition fall within 25% of the median diameter of the lipid particles. In some embodiments, 90% of the lipid particles in a composition fall within 20% of the median diameter. In some embodiments, 90% of the lipid particles in a composition fall within 15% of the median diameter of lipid particles. In some embodiments, 90% of the lipid particles in a composition fall within 10% of the median diameter of the lipid particles.

In some embodiments, 75% of the lipid particles in a composition fall within +/−2 or +/−1 St Dev standard deviations (St Dev) of the mean diameter of lipid particles. In some embodiments, 80% of the lipid particles in a composition fall within +/−2 St Dev or +/−1 St Dev of the mean diameter of lipid particles. In some embodiments, 85% of the lipid particles in a composition fall within +/−2 St Dev or +/−1 St Dev of the mean diameter of lipid particles. In some embodiments, 90% of the lipid particles in a composition fall within +/−2 St Dev or +/−1 St Dev of the mean diameter of lipid particles. In some embodiments, 95% of the lipid particles in a composition fall within +/−2 St Dev or +/−1 St Dev of the mean diameter of lipid particles.

In some embodiments, the lipid particles have an average hydrodynamic radius, e.g. as determined by DLS, of about 100 nm to about two microns. In some embodiments, the lipid particles have an average hydrodynamic radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the lipid particles have an average geometric radius, e.g. as determined by a multi-angle light scattering, of about 100 nm to about two microns. In some embodiments, the lipid particles have an average geometric radius between at or about 2 μm and at or about 1 μm, between at or about 1 μm and at or about 900 nm, between at or about 900 nm and at or about 800 nm, between at or about 800 and at or about 700 nm, between at or about 700 nm and at or about 600 nm, between at or about 600 nm and at or about 500 nm, between at or about 500 nm and at or about 400 nm, between at or about 400 nm and at or about 300 nm, between at or about 300 nm and at or about 200 nm, between at or about 200 and at or about 100 nm, between at or about 100 and at or about 50 nm, or between at or about 20 nm and at or about 50 nm.

In some embodiments, the coefficient of variation (COV) (i.e. standard deviation divided by the mean) of a composition of lipid particles is less than at or about 30%, less than at or about 25%, less than at or about 20%, less than at or about 15%, less than at or about 10% or less than at or about 5%.

In some embodiment, provided compositions of lipid particles are characterized by their polydispersity index, which is a measure of the size distribution of the particles wherein values between 1 (maximum dispersion) and 0 (identical size of all of the particles) are possible. In some embodiments, compositions of lipid particles provided herein have a polydispersity index of between at or about 0.05 and at or about 0.7, between at or about 0.05 and at or about 0.6, between at or about 0.05 and at or about 0.5, between at or about 0.05 and at or about 0.4, between at or about 0.05 and at or about 0.3, between at or about 0.05 and at or about 0.2, between at or about 0.05 and at or about 0.1, between at or about 0.1 and at or about 0.7, between at or about 0.1 and at or about 0.6, between at or about 0.1 and at or about 0.5, between at or about 0.1 and at or about 0.4, between at or about 0.1 and at or about 0.3, between at or about 0.1 and at or about 0.2, between at or about 0.2 and at or about 0.7, between at or about 0.2 and at or about 0.6, between at or about 0.2 and at or about 0.5, between at or about 0.2 and at or about 0.4 between at or about 0.2 and at or about 0.3, between at or about 0.3 and at or about 0.7, between at or about 0.3 and at or about 0.6, between at or about 0.3 and at or about 0.5, between at or about 0.3 and at or about 0.4, between at or about 0.4 and at or about 0.7, between at or about 0.4 and at or about 0.6, between at or about 0.4 and at or about 0.5, between at or about 0.5 and at or about 0.7, between at or about 0.5 and at or about 0.6, or between at or about 0.6 and at or about 0.7. In some embodiments, the polydispersity index is less than at or about 0.05, less than at or about 0.1, less than at or about 0.15, less than at or about 0.2, less than at or about 0.25, less than at or about 0.3, less than at or about 0.4, less than at or about 0.5, less than at or about 0.6 or less than at or about 0.7. Various lipid particles are known, any of which can be generated in accord with the provided embodiments. Non-limiting examples of lipid particles include any as described in, or contain features as described in, International published PCT Application No. WO 2017/095946; WO 2017/095944; WO 2017/095940; WO 2019/157319; WO 2018/208728; WO 2019/113512; WO 2019/161281; WO 2020/102578; WO 2019/222403; WO 2020/014209; WO 2020/102485; WO 2020/102499; WO 2020/102503; WO 2013/148327; WO 2017/182585; WO 2011/058052; or WO 2017/068077, each of which are incorporated by reference in their entirety.

Features of exemplary provided lipid particles are described in the following subsections.

### A. Viral-Based Particles

Provided herein are viral-based particles derived from a virus, including those derived from retroviruses or lentiviruses, containing a truncated BaEV envelope protein, such as described in Section I. In some embodiments, the lipid particle's bilayer of amphipathic lipids is or comprises the viral envelope. In some embodiments, the lipid particle's bilayer of amphipathic lipids is or comprises lipids derived from a producer cell. In some embodiments, the viral envelope may comprise a fusogen, e.g., a fusogen that is endogenous to the virus or a pseudotyped fusogen. In some embodiments, the lipid particle's lumen or cavity comprises a viral nucleic acid, e.g., a retroviral nucleic acid, e.g., a lentiviral nucleic acid. In some embodiments, the viral nucleic acid may be a viral genome. In some embodiments, the lipid particle further comprises one or more viral non-structural proteins, e.g., in its cavity or lumen. In some embodiments, the viral-based particle is or comprises a virus-like particle (VLP). In some embodiments, the VLP does not comprise any viral genetic material. In some embodiments, the viral-based particle does not contain any virally derived nucleic acids or viral proteins, such as viral structural proteins.

Biological methods for introducing an exogenous agent to a host cell include the use of DNA and RNA vectors. DNA and RNA vectors can also be used to house and deliver polynucleotides and polypeptides. Viral vectors and virus like particles, and especially retroviral vectors, have become the most widely used method for inserting genes into mammalian, e.g., human cells. Other viral vectors and virus like particles can be derived from lentivirus, poxviruses, herpes simplex virus I, adenoviruses and adeno-associated viruses, and the like. See, for example, U.S. Pat. Nos. 5,350,674 and 5,585,362. Methods for producing cells comprising vectors and/or exogenous acids are well-known in the art. See, for example, Sambrook et al., 2001, Molecular Cloning: A Laboratory Manual, Cold Spring Harbor Laboratory, New York.

In some embodiments, the viral particles or virus-like particles bilayer of amphipathic lipids is or comprises lipids derived from an infected host cell. In some embodiments, the lipid bilayer is a viral envelope. In some embodiments, the viral particles or virus-like particles envelope is obtained from a host cell. In some embodiments, the viral particles or virus-like particles envelope is obtained by the viral capsid from the source cell plasma membrane. In some embodiments, the lipid bilayer is obtained from a membrane other than the plasma membrane of a host cell. In some embodiments, the viral particles or virus-like particles envelope lipid bilayer is embedded with viral proteins, including viral glycoproteins.

In some embodiments, one or more transducing units of viral particles or virus-like particles, e.g. retroviral particles or retroviral-like particles, are administered to the subject. In some embodiments, at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per kg are administered to the subject. In some embodiments at least 1, 10, 100, 1000, 10⁴, 10⁵, 10⁶, 10⁷, 10⁸, 10⁹, 10¹⁰, 10¹¹, 10¹², 10¹³, or 10¹⁴, transducing units per target cell per ml of blood are administered to the subject.

### 1. Viral Vector Particles

In some embodiments, the lipid particle is or comprises a virus or a viral vector, e.g., a retrovirus or retroviral vector, e.g., a lentivirus or lentiviral vector. In some embodiments, the virus or viral vector is recombinant. For instance, the viral particle may be referred to as a recombinant virus or a recombinant viral vector, which are used interchangeably. In some embodiments, the lipid particle is a recombinant lentivirus vector particle.

In some embodiments, a lipid particle comprises a lipid bilayer comprising a retroviral vector comprising an envelope. For instance, in some embodiments, the bilayer of amphipathic lipids is or comprises the viral envelope. The viral envelope may comprise a fusogen, e.g., a truncated BaEV fusogen, that is endogenous to the virus or is a pseudotyped fusogen. In some embodiments, the viral vector's lumen or cavity comprises a viral nucleic acid, e.g., a retroviral nucleic acid, e.g., a lentiviral nucleic acid. The viral nucleic acid may be a viral genome. In some embodiments, the viral vector may further comprises one or more viral non-structural proteins, e.g., in its cavity or lumen. In some embodiments, the virus based vector particles are lentivirus. In some embodiments, the lentiviral vector particle is Human Immunodeficiency Virus-1 (HIV-1).

In some aspects, the viral vector particle is limited in the number of polynucleotides that can be packaged. In some embodiments, nucleotides encoding polypeptides to be packaged can be modified such that they retain functional activity with fewer nucleotides in the coding region than that which encodes for the wild-type peptide. Such modifications can include truncations, or other deletions. In some embodiments, more than one polypeptide can be expressed from the same promoter, such that they are fusion polypeptides. In some embodiments, the insert size to be packaged (i.e., viral genome, or portions thereof; or heterologous polynucleotides as described) can be between 500-1000, 1000-2000, 2000-3000, 3000-4000, 4000-5000, 5000-6000, 6000-7000, or 7000-8000 nucleotides in length. In some embodiments, the insert can be over 8000 nucleotides, such as 9000, 10,000, or 11,000 nucleotides in length.

In some embodiments, the viral vector particle, such as retroviral vector particle, comprises one or more of gag polyprotein, polymerase (e.g., pol), integrase (e.g., a functional or non-functional variant), protease, and a fusogen. In some embodiments, the lipid particle further comprises rev. In some embodiments, one or more of the aforesaid proteins are encoded in the retroviral genome (i.e., the insert as described above), and in some embodiments, one or more of the aforesaid proteins are provided in trans, e.g., by a helper cell, helper virus, or helper plasmid. In some embodiments, the lipid particle nucleic acid (e.g., retroviral nucleic acid) comprises one or more of the following nucleic acid sequences: 5′ LTR (e.g., comprising U5 and lacking a functional U3 domain), Psi packaging element (Psi), Central polypurine tract (cPPT) Promoter operatively linked to the payload gene, payload gene (optionally comprising an intron before the open reading frame), Poly A tail sequence, WPRE, and 3′ LTR (e.g., comprising U5 and lacking a functional U3). In some embodiments, the lipid particle nucleic acid further comprises a retroviral cis-acting RNA packaging element, and a cPPT/CTS element. In some embodiments the lipid particle nucleic acid further comprises one or more insulator element. In some embodiments, the recognition sites are situated between the poly A tail sequence and the WPRE.

In some embodiments, the lipid particle comprises supramolecular complexes formed by viral proteins that self-assemble into capsids. In some embodiments, the lipid particle is a viral particle derived from viral capsids. In some embodiments, the lipid particle is a viral particle derived from viral nucleocapsids. In some embodiments, the lipid particle comprises nucleocapsid-derived that retain the property of packaging nucleic acids.

In some embodiments, the lipid particle packages nucleic acids from host cells carrying one or more viral nucleic acids (e.g. retroviral nucleic acids) during the expression process. In some embodiments, the nucleic acids do not encode any genes involved in virus replication. In particular embodiments, the lipid particle is a virus-based particle, e.g. retrovirus particle such as a lentivirus particle, that is replication defective.

In some cases, the lipid particle is a viral particle that is morphologically indistinguishable from the wild type infectious virus. In some embodiments, the viral particle presents the entire viral proteome as an antigen. In some embodiments, the viral particle presents only a portion of the proteome as an antigen.

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT, a WPRE, and/or an insulator element.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretro virus. In some embodiments the retrovirus is a Deltaretro virus. In some embodiments the retrovirus is a Lentivirus. In some embodiments the retrovirus is a Spumaretrovirus. In some embodiments the retrovirus is an endogenous retrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

A viral vector can comprise a nucleic acid molecule (e.g., a transfer plasmid) that includes virus-derived nucleic acid elements that typically facilitate transfer of a nucleic acid molecule (e.g. including nucleic acid encoding an exogenous agent) or integration into the genome of a cell or to a viral particle that mediates nucleic acid transfer. Viral vector particles will typically include various viral components and sometimes also host cell components in addition to nucleic acid(s). A viral vector can comprise a virus or viral particle capable of transferring a nucleic acid into a cell (e.g. nucleic acid encoding an exogenous agent), or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses.

For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex. With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome gag, pol and env may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein.

In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell, such as nucleic acid encoding an exogenous agent as described herein. In embodiments, an RLV is incapable of independent replication to produce infectious retroviral particles within the target cell. Usually the RLV lacks a functional gag-pol and/or env gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′). However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non-dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both.

In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMHD1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

In some embodiments, codon optimization has a number of other advantages. In some embodiments, by virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

In some embodiments, derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

In some embodiments, due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

It is within the level of a skilled artisan to empirically determine appropriate codon optimization of viral sequences. The strategy for codon optimized sequences, including gag-pol sequences, can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV and human endogenous retroviruses (HERV), MLV and other retroviruses.

In embodiments, the retroviral vector comprises a packaging signal that comprises from 255 to 360 nucleotides of gag in vectors that still retain env sequences, or about 40 nucleotides of gag in a particular combination of splice donor mutation, gag and env deletions. In some embodiments, the retroviral vector includes a gag sequence which comprises one or more deletions, e.g., the gag sequence comprises about 360 nucleotides derivable from the N-terminus.

In some embodiments, the retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

In some embodiments, the gag and pol coding sequences are generally organized as the Gag-Pol Precursor in native lentivirus. The gag sequence codes for a 55-kD Gag precursor protein, also called p55. The p55 is cleaved by the virally encoded protease (a product of the pol gene) during the process of maturation into four smaller proteins designated MA (matrix [p17]), CA (capsid [p24]), NC (nucleocapsid [p9]), and p6. The pol precursor protein is cleaved away from Gag by a virally encoded protease, and further digested to separate the protease (p10), RT (p50), RNase H (p15), and integrase (p31) activities.

In some embodiments, the lentiviral vector is integration-deficient. In some embodiments, the pol is integrase deficient, such as by encoding due to mutations in the integrase gene. For example, the pol coding sequence can contain an inactivating mutation in the integrase, such as by mutation of one or more of amino acids involved in catalytic activity, i.e. mutation of one or more of aspartic 64, aspartic acid 116 and/or glutamic acid 152. In some embodiments, the integrase mutation is a D64V mutation. In some embodiments, the mutation in the integrase allows for packaging of viral RNA into a lentivirus. In some embodiments, the mutation in the integrase allows for packaging of viral proteins into a lentivirus. In some embodiments, the mutation in the integrase reduces the possibility of insertional mutagenesis. In some embodiments, the mutation in the integrase decreases the possibility of generating replication-competent recombinants (RCRs) (Wanisch et al. 2009. Mol Ther. 1798): 1316-1332). In some embodiments, native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In some embodiments, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS1, (ii) contains two nucleotide insertion that results in frame shift and premature termination, and/or (iii) does not include INS2, INS3, and INS4 inhibitory sequences of gag.

In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-1. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et al., (1997); Dull et al., 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

In some embodiments, a packaging signal can comprise a sequence located within the retroviral genome which mediate insertion of the viral RNA into the viral capsid or particle, see e.g., Clever et al., 1995. J. of Virology, Vol. 69, No. 4; pp. 2101-2109. Several retroviral vectors use a minimal packaging signal (a psi [Y] sequence) for encapsidation of the viral genome.

In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. In some aspects, provided herein is a replication incompetent (also referred to herein as replication defective) vector particle, that cannot participate in replication in the absence of the packaging cell (i.e., viral vector particles are not produced from the transduced cell). In some aspects, this is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs. Other modifications to the viral vector, i.e., retroviral or lentiviral vector, to render said vector replication incompetent are known in the art.

In some embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. For example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et ah, 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et al., 1991. J. Virol. 65: 1053; and Cullen et al., 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences (e.g. nucleic acid encoding an exogenous agent) in viral vectors is increased by incorporating one or more of, e.g., all of, posttranscriptional regulatory elements, polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II. Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA, ATT AAA, AGTAAA, a bovine growth hormone polyA sequence (BGHpA), a rabbit b-globin polyA sequence (rPgpA), or another suitable heterologous or endogenous polyA sequence.

In some embodiments, a retroviral or lentiviral vector further comprises one or more insulator elements, e.g., an insulator element described herein.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### 2. Virus-Like Vector Particle

In some embodiments, the viral-based particles are viral-like lipid particles (VLPs) that are derived from virus. In some embodiments, the viral envelope may comprise a fusogen, e.g., a fusogen that is endogenous to the virus or a pseudotyped fusogen. The VLPS include those derived from retroviruses or lentiviruses. While VLPs mimic native virion structure, they lack the viral genomic information necessary for independent replication within a host cell. Therefore, in some aspects, VLPs are non-infectious. In particular embodiments, a VLP does not contain a viral genome. In some embodiments, the VLP's bilayer of amphipathic lipids is or comprises the viral envelope. In some embodiments, the targeted lipid particle's bilayer of amphipathic lipids is or comprises lipids derived from a cell. In some embodiments, a VLP contains at least one type of structural protein from a virus. In most cases this protein will form a proteinaceous capsid. In some cases the capsid will also be enveloped in a lipid bilayer originating from the cell from which the assembled VLP has been released (e.g. VLPs comprising a human immunodeficiency virus structural protein such as GAG). In some embodiments, the VLP further comprises a targeting moiety as an envelope protein within the lipid bilayer.

In some embodiments, the vector vehicle particle comprises supramolecular complexes formed by viral proteins that self-assemble into capsids. In some embodiments, the vector vehicle particle is a virus-like particle derived from viral capsid proteins. In some embodiments, the vector vehicle particle is a virus-like particle derived from viral nucleocapsid proteins. In some embodiments, the vector vehicle particle comprises nucleocapsid-derived proteins that retain the property of packaging nucleic acids. In some embodiments, the viral-based particles, such as virus-like particles comprises only viral structural glycoproteins among proteins from the viral genome. In some embodiments, the vector vehicle particle does not contain a viral genome.

In some embodiments, the vector vehicle particle packages nucleic acids from host cells during the expression process, such as a nucleic acid encoding an exogenous agent. In some embodiments, the nucleic acids do not encode any genes involved in virus replication. In particular embodiments, the vector vehicle particle is a virus-like particle, e.g. retrovirus-like particle such as a lentivirus-like particle, that is replication defective.

In some embodiments, the vector vehicle particle is a virus-like particle which comprises a sequence that is devoid of or lacking viral RNA may be the result of removing or eliminating the viral RNA from the sequence. In some embodiments, this may be achieved by using an endogenous packaging signal binding site on gag. In some embodiments, the endogenous packaging signal binding site is on pol. In some embodiments, the RNA which is to be delivered will contain a cognate packaging signal. In some embodiments, a heterologous binding domain (which is heterologous to gag) located on the RNA to be delivered, and a cognate binding site located on gag or pol, can be used to ensure packaging of the RNA to be delivered. In some embodiments, the heterologous sequence could be non-viral or it could be viral, in which case it may be derived from a different virus. In some embodiments, the vector particles could be used to deliver therapeutic RNA, in which case functional integrase and/or reverse transcriptase is not required. In some embodiments, the vector particles could also be used to deliver a therapeutic gene of interest, in which case pol is typically included.

In some embodiments, the VLP comprises supramolecular complexes formed by viral proteins that self-assemble into capsids. In some embodiments, the VLP is derived from viral capsids. In some embodiments, the VLP is derived from viral nucleocapsids. In some embodiments, the VLP is nucleocapsid-derived and retains the property of packaging nucleic acids. In some embodiments, the VLP includes only viral structural glycoproteins. In some embodiments, the VLP does not contain a viral genome.

### 3. Methods of Generating Viral-Based Particles

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In some embodiments, viral vector particles may be produced in multiple cell culture systems including bacteria, mammalian cell lines, insect cell lines, yeast and plant cells. Exemplary methods for producing viral vector particles are described.

In some embodiments, elements for the production of a viral vector, i.e., a recombinant viral vector such as a replication incompetent lentiviral vector, are included in a packaging cell line or are present on a packaging vector. In some embodiments, viral vectors can include packaging elements, rev, gag, and pol, delivered to the packaging cells line via one or more packaging vectors.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides. In some embodiments, the packaging vector is a packaging plasmid.

Producer cell lines (also called packaging cell lines) include cell lines that do not contain a packaging signal, but do stably or transiently express viral structural proteins and replication enzymes (e.g., gag, pol and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells. In embodiments, the packaging cells are 293 cells, 293T cells, or A549 cells.

In some embodiments, a producer cell (i.e., a source cell line) includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

In some embodiments, the source cell comprises one or more plasmids coding for viral structural proteins and replication enzymes (e.g., gag, pol and env) which can package viral particles (i.e, a packaging plasmid). In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome.

In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

Typically, modern retroviral vector systems include viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A virus-like particle (VLP) which comprises a sequence that is devoid of or lacking viral RNA as described in Section III. A.2 may be the result of removing or eliminating the viral RNA from the sequence. Similar to the viral vector particles disclosed in Section III.A.1, VLPs contain a viral outer envelope made from the host cell (i.e., producer cell or source cell) lipid-bi layer as well as at least one viral structural protein. In some embodiments, a viral structural protein refers to any viral protein or fragment thereof which contributes to the structure of the viral core or capsid.

Generally, for viral vector particles as described in Section III.A.1, expression of the gag precursor protein alone mediates vector assembly and release. In some aspects, gag proteins or fragments thereof have been demonstrated to assemble into structures analogous to viral cores. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The VLP could be used to deliver therapeutic RNA, in which case functional integrase and/or reverse transcriptase is not required. These VLPs could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognizing a corresponding sequence in an RNA sequence to facilitate packaging of the RNA sequence into a viral vector particle. In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, the assembly of a viral based vector vehicle particle (i.e., a VLP) is initiated by binding of the core protein to a unique encapsidation sequence within the viral genome (e.g. UTR with stem-loop structure). In some embodiments, the interaction of the core with the encapsidation sequence facilitates oligomerization.

In some embodiments, the source cell for VLP production comprises one or more plasmids coding for viral structural proteins (e.g., gag, pol) which can package viral particles (i.e., a packaging plasmid). In some embodiments, the sequences coding for at least two of the gag and pol precursors are on the same plasmid. In some embodiments, the sequences coding for the gag and pol precursors are on different plasmids. In some embodiments, the sequences coding for the gag and pol precursors have the same expression signal, e.g., promoter. In some embodiments, the sequences coding for the gag and pol precursors have a different expression signal, e.g., different promoters. In some embodiments, expression of the gag and pol precursors is inducible.

In some embodiments, formation of VLPs or any viral-based particle as described above in Section III can be detected by any suitable technique known in the art. Examples of such techniques include, e.g., electron microscopy, dynamic light scattering, selective chromatographic separation and/or density gradient centrifugation.

### B. Cell Based Particles

In some embodiments, the lipid particle is a cell based particle that comprises a naturally derived membrane. In some embodiments, the naturally derived membrane comprises membrane vesicles prepared from cells or tissues. In some embodiments, the cell based particle comprises a vesicle that is obtainable from a cell. In some embodiments, the cell based particle comprises a microvesicle, an exosome, a membrane enclosed body, an apoptotic body (from apoptotic cells), a particle (which may be derived from e.g. platelets), an ectosome (derivable from, e.g., neutrophiles and monocytes in serum), a prostatosome (obtainable from prostate cancer cells), or a cardiosome (derivable from cardiac cells).

In some embodiments, the source cell is an endothelial cell, a fibroblast, a blood cell (e.g., a macrophage, a neutrophil, a granulocyte, a leukocyte), a stem cell (e.g., a mesenchymal stem cell, an umbilical cord stem cell, bone marrow stem cell, a hematopoietic stem cell, an induced pluripotent stem cell e.g., an induced pluripotent stem cell derived from a subject's cells), an embryonic stem cell (e.g., a stem cell from embryonic yolk sac, placenta, umbilical cord, fetal skin, adolescent skin, blood, bone marrow, adipose tissue, erythropoietic tissue, hematopoietic tissue), a myoblast, a parenchymal cell (e.g., hepatocyte), an alveolar cell, a neuron (e.g., a retinal neuronal cell) a precursor cell (e.g., a retinal precursor cell, a myeloblast, myeloid precursor cells, a thymocyte, a meiocyte, a megakaryoblast, a promegakaryoblast, a melanoblast, a lymphoblast, a bone marrow precursor cell, a normoblast, or an angioblast), a progenitor cell (e.g., a cardiac progenitor cell, a satellite cell, a radial gial cell, a bone marrow stromal cell, a pancreatic progenitor cell, an endothelial progenitor cell, a blast cell), or an immortalized cell (e.g., HeEa, HEK293, HFF-1, MRC-5, WI-38, IMR 90, IMR 91, PER.C6, HT-1080, or BJ cell). In some embodiments, the source cell is other than a 293 cell, HEK cell, human endothelial cell, or a human epithelial cell, monocyte, macrophage, dendritic cell, or stem cell.

In some embodiments, the cell based particle has a density of <1, 1-1.1, 1.05-1.15, 1.1-1.2, 1.15-1.25, 1.2-1.3, 1.25-1.35, or >1.35 g/ml. In some embodiments, the vector vehicle particle composition comprises less than 0.01%, 0.05%, 0.1%, 0.5%, 1%, 1.5%, 2%, 2.5%, 3%, 4%, 5%, or 10% source cells by protein mass or less than 0.01%, 0.05%, 0.1%, 0.5%, 1%, 1.5%, 2%, 2.5%, 3%, 4%, 5%, or 10% of cells having a functional nucleus.

In embodiments, the cell based particle has a size, or the population of vector vehicle particles have an average size, that is less than about 0.01%, 0.05%, 0.1%, 0.5%, 1%, 2%, 3%, 4%, 5%, 10%, 20%, 30%, 40%, 50%, 60%, 70%, 80%, 90%, of that of the source cell.

In some embodiments the cell based particle is an extracellular vesicle, e.g., a cell based vesicle comprising a membrane that encloses an internal space and has a smaller diameter than the cell from which it is derived. In embodiments the extracellular vesicle has a diameter from 20 nm to 1000 nm. In embodiments the cell based particle is an apoptotic body, a fragment of a cell, a vesicle derived from a cell by direct or indirect manipulation, a vesiculated organelle, and a vesicle produced by a living cell (e.g., by direct plasma membrane budding or fusion of the late endosome with the plasma membrane). In embodiments the extracellular vesicle is derived from a living or dead organism, explanted tissues or organs, or cultured cells.

In embodiments, the cell based particle is a nanovesicle, e.g., a cell-derived small (e.g., between 20-250 nm in diameter, or 30-150 nm in diameter) vesicle comprising a membrane that encloses an internal space, and which is generated from said cell by direct or indirect manipulation. The production of nanovesicles can, in some instances, result in the destruction of the source cell. The nanovesicle may comprise a lipid or fatty acid and polypeptide.

In embodiments, the cell based particle is an exosome. In embodiments, the exosome is a cell-derived small (e.g., between 20-300 nm in diameter, or 40-200 nm in diameter) vesicle comprising a membrane that encloses an internal space, and which is generated from said cell by direct plasma membrane budding or by fusion of the late endosome with the plasma membrane. In embodiments, production of exosomes does not result in the destruction of the source cell. In embodiments, the exosome comprises lipid or fatty acid and polypeptide. Exemplary exosomes and other membrane-enclosed bodies are also described in WO/2017/161010, WO/2016/077639, US20160168572, US20150290343, and US20070298118, each of which is incorporated by reference herein in its entirety.

In some embodiments, the cell based particle is a microvesicle. In some embodiments the microvesicle has a diameter of about 100 nm to about 2000 nm.

In some embodiments, a the cell based particle is a cell ghost. In some embodiments, a vesicle is a plasma membrane vesicle, e.g. a giant plasma membrane vesicle.

In some embodiments, the cell based particle is derived from a source cell with a genetic modification which results in increased expression of an immunomodulatory agent, such as an immunosuppressive agent. In some embodiments, the immunosuppressive agent is on an exterior surface of the cell. In some embodiments, the immunosuppressive agent is incorporated into the exterior surface of the vector vehicle particle. In some embodiments, the vector vehicle particle comprises an immunomodulatory agent attached to the surface of the solid particle by a covalent or non-covalent bond

In some embodiments, cell based particles are generated by inducing budding of an exosome, microvesicle, membrane vesicle, extracellular membrane vesicle, plasma membrane vesicle, giant plasma membrane vesicle, apoptotic body, mitoparticle, pyrenocyte, lysosome, or other membrane enclosed vesicle.

In some embodiments, cell based particles are generated by inducing cell enucleation. Enucleation may be performed using assays such as genetic, chemical (e.g., using Actinomycin D, see Bayona-Bafaluy et al., “A chemical enucleation method for the transfer of mitochondrial DNA to ρ° cells” Nucleic Acids Res. 2003 Aug. 15; 31(16): e98), mechanical methods (e.g., squeezing or aspiration, see Lee et al., “A comparative study on the efficiency of two enucleation methods in pig somatic cell nuclear transfer: effects of the squeezing and the aspiration methods.” Anim Biotechnol. 2008; 19(2):71-9), or combinations thereof.

In some embodiments, the cell based particles are generated by inducing cell fragmentation. In some embodiments, cell fragmentation can be performed using the following methods, including, but not limited to: chemical methods, mechanical methods (e.g., centrifugation (e.g., ultracentrifugation, or density centrifugation), freeze-thaw, or sonication), or combinations thereof.

In some embodiments, the source cell used to make the cell based particle will not be available for testing after the vector vehicle particle is made.

In some embodiments, a characteristic of a cell based particle is described by comparison to a reference cell. In embodiments, the reference cell is the source cell. In embodiments, the reference cell is a HeLa, HEK293, HFF-1, MRC-5, WI-38, IMR 90, IMR 91, PER.C6, HT-1080, or BJ cell. In some embodiments, a characteristic of a population of vector vehicle particles is described by comparison to a population of reference cells, e.g., a population of source cells, or a population of HeLa, HEK293, HFF-1, MRC-5, WI-38, IMR 90, IMR 91, PER.C6, HT-1080, or BJ cells.

### C. Exogenous Agent

In some embodiments, the lipid particle or pharmaceutical composition comprising same described herein contains an exogenous agent. In some embodiments, the lipid particle or pharmaceutical composition comprising same described herein contains a nucleic acid that encodes an exogenous agent. In some embodiments, the lipid particle contains the exogenous agent. In some embodiments, the lipid particle contains a nucleic acid that encodes an exogenous agent. Reference to the coding sequence of the nucleic acid encoding the exogenous agent also is referred to herein as a payload gene. In some embodiments, the exogenous agent or the nucleic acid encoding the exogenous agent are present in the lumen of the lipid particle.

In some embodiments, the exogenous agent is a protein or a nucleic acid (e.g., a DNA, a chromosome (e.g. a human artificial chromosome), an RNA, e.g., an mRNA or miRNA). In some embodiments, the exogenous agent is a protein. In some embodiments, the exogenous agent is a nucleic acid (e.g., a DNA, a chromosome (e.g. a human artificial chromosome), an RNA, e.g., an mRNA or miRNA). In some embodiments, the exogenous agent comprises or encodes a membrane protein. In some embodiments, the exogenous agent comprises or encodes a therapeutic agent. In some embodiments, the therapeutic agent is chosen from one or more of a protein, e.g., an enzyme, a transmembrane protein, a receptor, or an antibody; a nucleic acid, e.g., DNA, a chromosome (e.g. a human artificial chromosome), RNA, mRNA, siRNA, or miRNA; or a small molecule.

In some embodiments, the lipid particle or pharmaceutical composition delivers to a target cell at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle. In some embodiments, the lipid particle, e.g., fusosome, that contacts, e.g., fuses, with the target cell(s) delivers to the target cell an average of at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particles, e.g., fusosomes, that contact, e.g., fuse, with the target cell(s). In some embodiments, the lipid particle composition delivers to a target tissue at least 10%, 20%, 30%, 40%, 50%, 60%, 70%, 80%, 90%, 95%, 96%, 97%, 98%, or 99% of the exogenous agent (e.g., an exogenous agent comprising or encoding a therapeutic agent) comprised by the lipid particle compositions.

In some embodiments, the exogenous agent is not expressed naturally in the cell from which the lipid particle is derived. In some embodiments, the exogenous agent is expressed naturally in the cell from which the lipid particle is derived. In some embodiments, the exogenous agent is loaded into the lipid particle via expression in the cell from which the lipid particle is derived (e.g. expression from DNA or mRNA introduced via transfection, transduction, or electroporation). In some embodiments, the exogenous is expressed from DNA integrated into the genome or maintained episomally. In some embodiments, expression of the exogenous agent is constitutive. In some embodiments, expression of the exogenous agent is induced. In some embodiments, expression of the exogenous agent is induced immediately prior to generating the lipid particle. In some embodiments, expression of the exogenous agent is induced at the same time as expression of the fusogen.

In some embodiments, the exogenous agent is loaded into the lipid particle via electroporation into the lipid particle itself or into the cell from which the lipid particle is derived. In some embodiments, the exogenous agent is loaded into the lipid particle via transfection (e.g., of a DNA or mRNA encoding the exogenous agent) into the lipid particle itself or into the cell from which the lipid particle is derived.

In some embodiments, the exogenous agent may include one or more nucleic acid sequences, one or more polypeptides, a combination of nucleic acid sequences and/or polypeptides, one or more organelles, and any combination thereof. In some embodiments, the exogenous agent may include one or more cellular components. In some embodiments, the exogenous agent includes one or more cytosolic and/or nuclear components.

In some embodiments, the lipid particle contains an exogenous agent that is a nucleic acid or contains a nucleic acid encoding the exogenous agent. In some embodiments, the nucleic acid is operatively linked to a “positive target cell-specific regulatory element” (or positive TCSRE). In some embodiments, the positive TCSRE is a functional nucleic acid sequence. In some embodiments, the positive TCSRE comprises a promoter or enhancer. In some embodiments, the TCSRE is a nucleic acid sequence that increases the level of an exogenous agent in a target cell. In some embodiments, the positive target cell-specific regulatory element comprises a T cell-specific promoter, a T cell-specific enhancer, a T cell-specific splice site, a T cell-specific site extending half-life of an RNA or protein, a T cell-specific mRNA nuclear export promoting site, a T cell-specific translational enhancing site, or a T cell-specific post-translational modification site. In some embodiments, the T cell-specific promoter is a promoter described in Immgen consortium, herein incorporated by reference in its entirety, e.g., the T cell-specific promoter is an IL2RA (CD25), LRRC32, FOXP3, or IKZF2 promoter. In some embodiments, the T cell-specific promoter or enhancer is a promoter or enhancer described in Schmidl et a, Blood. 2014 Apr. 24; 123(17):e68-78., herein incorporated by reference in its entirety. In some embodiments, the T cell-specific promoter is a transcriptionally active fragment of any of the foregoing. In some embodiments, the T-cell specific promoter is a variant having at least 70%, 75%, 80%, 85%, 90%, 95%, 96%, 97%, 98%, or 99% identity to any of the foregoing.

In some embodiments, the lipid particle contains an exogenous agent that is a nucleic acid or contains a nucleic acid encoding the exogenous agent. In some embodiments, the nucleic acid is operatively linked to a “negative target cell-specific regulatory element” (or negative TCSRE). In some embodiments, the negative TCSRE is a functional nucleic acid sequence. In some embodiments, the negative TCSRE is a miRNA recognition site that causes degradation of inhibition of the lipid particle in a non-target cell. In some embodiments, the exogenous agent is operatively linked to a “non-target cell-specific regulatory element” (or NTCSRE). In some embodiments, the NTCSRE comprises a nucleic acid sequence that decreases the level of an exogenous agent in a non-target cell compared to in a target cell. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a tissue-specific miRNA recognition sequence, tissue-specific protease recognition site, tissue-specific ubiquitin ligase site, tissue-specific transcriptional repression site, or tissue-specific epigenetic repression site. In some embodiments, the NTCSRE comprises a non-target cell-specific miRNA recognition sequence, non-target cell-specific protease recognition site, non-target cell-specific ubiquitin ligase site, non-target cell-specific transcriptional repression site, or non-target cell-specific epigenetic repression site. In some embodiments, the NTCSRE comprises a non-target cell-specific miRNA recognition sequence and the miRNA recognition sequence is able to be bound by one or more of miR3 1, miR363, or miR29c. In some embodiments, the NTCSRE is situated or encoded within a transcribed region encoding the exogenous agent, optionally wherein an RNA produced by the transcribed region comprises the miRNA recognition sequence within a UTR or coding region.

### 1. Nucleic Acids

In some embodiments, the exogenous agent may include a nucleic acid. For example, the exogenous agent may comprise RNA to enhance expression of an endogenous protein, or a siRNA or miRNA that inhibits protein expression of an endogenous protein. For example, the endogenous protein may modulate structure or function in the target cells. In some embodiments, the exogenous agent may include a nucleic acid encoding an engineered protein that modulates structure or function in the target cells. In some embodiments, the exogenous agent is a nucleic acid that targets a transcriptional activator that modulate structure or function in the target cells

In some embodiments, a lipid particle described herein comprises a nucleic acid, e.g., RNA or DNA. In some embodiments, the nucleic acid is, comprises, or consists of one or more natural nucleic acid residues. In some embodiments, the nucleic acid is, comprises, or consists of one or more nucleic acid analogs. In some embodiments, the nucleic acid has a nucleotide sequence that encodes a functional gene product such as an RNA or protein. In some embodiments, the nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, the nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, the nucleic acid is partly or wholly single stranded; in some embodiments, the nucleic acid is partly or wholly double stranded. In some embodiments the nucleic acid has a nucleotide sequence comprising at least one element that encodes, or is the complement of a sequence that encodes, a polypeptide. The nucleic acid may include variants, e.g., having an overall sequence identity with a reference nucleic acid of at least 85%, 86%, 87%, 88%, 89%, 90%, 91%, 92%, 93%, 94%, 95%, 96%, 97%, or 99%. In some embodiments, a variant nucleic acid does not share at least one characteristic sequence element with a reference nucleic acid. In some embodiments, a variant nucleic acid shares one or more of the biological activities of the reference nucleic acid. In some embodiments, a nucleic acid variant has a nucleic acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant nucleic acid comprises about 10, about 9, about 8, about 7, about 6, about 5, about 4, about 3, about 2, or about 1 substituted residue as compared to a reference. In some embodiments, a variant nucleic acid comprises a very small number (e.g., fewer than about 5, about 4, about 3, about 2, or about 1) number of substituted, inserted, or deleted, functional residues that participate in a particular biological activity relative to the reference. In some embodiments, a variant nucleic acid comprises not more than about 15, about 12, about 9, about 3, or about 1 addition or deletion, and, in some embodiments, comprises no additions or deletions, as compared to the reference. In some embodiments, a variant nucleic acid comprises fewer than about 27, about 24, about 21, about 18, about 15, about 12, about 9, about 6, about 3, or fewer than about 9, about 6, about 3, or about 2 additions or deletions as compared to the reference.

In some embodiments, the exogenous agent includes a nucleic acid, e.g., DNA, nDNA (nuclear DNA), mtDNA (mitochondrial DNA), protein coding DNA, gene, operon, chromosome, genome, transposon, retrotransposon, viral genome, intron, exon, modified DNA, mRNA (messenger RNA), tRNA (transfer RNA), modified RNA, microRNA, siRNA (small interfering RNA), tmRNA (transfer messenger RNA), IRNA (ribosomal RNA), mtRNA (mitochondrial RNA), snRNA (small nuclear RNA), small nucleolar RNA (snoRNA), SmY RNA (mRNA trans-splicing RNA), gRNA (guide RNA), TERC (telomerase RNA component), aRNA (antisense RNA), cis-NAT (Cis-natural antisense transcript), CRISPR RNA (crRNA), lncRNA (long noncoding RNA), piRNA (piwi-interacting RNA), shRNA (short hairpin RNA), tasiRNA (trans-acting siRNA), eRNA (enhancer RNA), satellite RNA, pcRNA (protein coding RNA), dsRNA (double stranded RNA), RNAi (interfering RNA), circRNA (circular RNA), reprogramming RNAs, aptamers, and any combination thereof. In some embodiments, the nucleic acid is a wild-type nucleic acid. In some embodiments, the protein is a mutant nucleic acid. In some embodiments the nucleic acid is a fusion or chimera of multiple nucleic acid sequences

In embodiments, the nucleic acid encodes one or more (e.g. two or more) inhibitory RNA molecules directed against one or more RNA targets. An inhibitory RNA molecule can be, e.g., a miRNA or an shRNA. In some embodiments, the inhibitory molecule can be a precursor of a miRNA, such as for example, a Pri-miRNA or a Pre-miRNA, or a precursor of an shRNA. In some embodiments, the inhibitory molecule can be an artificially derived miRNA or shRNA. In other embodiments, the inhibitory RNA molecule can be a dsRNA (either transcribed or artificially introduced) that is processed into an siRNA or the siRNA itself. In some embodiments, the inhibitory RNA molecule can be a miRNA or shRNA that has a sequence that is not found in nature, or has at least one functional segment that is not found in nature, or has a combination of functional segments that are not found in nature. In illustrative embodiments, at least one or all of the inhibitory RNA molecules are miR-155. In some embodiments, a retroviral vector described herein encodes two or more inhibitory RNA molecules directed against one or more RNA targets. Two or more inhibitory RNA molecules, in some embodiments, can be directed against different targets. In other embodiments, the two or more inhibitory RNA molecules are directed against the same target. In some embodiments, the exogenous agent comprises a shRNA. A shRNA (short hairpin RNA) can comprise a double-stranded structure that is formed by a single self complementary RNA strand. shRNA constructs can comprise a nucleotide sequence identical to a portion, of either coding or non-coding sequence, of a target gene. RNA sequences with insertions, deletions, and single point mutations relative to the target sequence can also be used. Greater than 90% sequence identity, or even 100% sequence identity, between the inhibitory RNA and the portion of the target gene can be used. In certain embodiments, the length of the duplex-forming portion of an shRNA is at least 20, 21 or 22 nucleotides in length, e.g., corresponding in size to RNA products produced by Dicer-dependent cleavage. In certain embodiments, the shRNA construct is at least 25, 50, 100, 200, 300 or 400 bases in length. In certain embodiments, the shRNA construct is 400-800 bases in length. shRNA constructs are highly tolerant of variation in loop sequence and loop size. In embodiments, a retroviral vector that encodes an siRNA, an miRNA, an shRNA, or a ribozyme comprises one or more regulatory sequences, such as, for example, a strong constitutive pol III, e.g., human U6 snRNA promoter, the mouse U6 snRNA promoter, the human and mouse H 1 RNA promoter and the human tRNA-val promoter, or a strong constitutive pol II promoter.

### 2. Polypeptides

In some embodiments, the lipid particle contains a nucleic acid that encodes a protein exogenous agent (also referred to as a “payload gene encoding an exogenous agent.”). In some embodiments, a lipid particle described herein comprises an exogenous agent which is or comprises a protein.

In some embodiments, the protein may include moieties other than amino acids (e.g., may be glycoproteins, proteoglycans, etc.) and/or may be otherwise processed or modified. In some embodiments, the protein can sometimes include more than one polypeptide chain, for example linked by one or more disulfide bonds or associated by other means.

In some embodiments, the protein may contain L-amino acids, D-amino acids, or both and may contain any of a variety of amino acid modifications or analogs. In some embodiments, proteins may comprise natural amino acids, non-natural amino acids, synthetic amino acids, and combinations thereof. In some embodiments, proteins are antibodies, antibody fragments, biologically active portions thereof, and/or characteristic portions thereof. In some embodiments, a polypeptide may include its variants, e.g., having an overall sequence identity with a reference polypeptide of at least 85%, 86%, 87%, 88%, 89%, 90%, 91%, 92%, 93%, 94%, 95%, 96%, 97%, or 99%. In some embodiments, a variant polypeptide does not share at least one characteristic sequence element with a reference polypeptide. In some embodiments, a variant polypeptide shares one or more of the biological activities of the reference polypeptide. In some embodiments, a polypeptide variant has an amino acid sequence that is identical to that of the reference but for a small number of sequence alterations at particular positions. In some embodiments, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide comprises about 10, about 9, about 8, about 7, about 6, about 5, about 4, about 3, about 2, or about 1 substituted residue as compared to a reference. In some embodiments, a variant polypeptide comprises a very small number (e.g., fewer than about 5, about 4, about 3, about 2, or about 1) number of substituted, inserted, or deleted, functional that participate in a particular biological activity relative to the reference. In some embodiments, a variant polypeptide comprises not more than about 5, about 4, about 3, about 2, or about 1 addition or deletion, and, in some embodiments, comprises no additions or deletions, as compared to the reference. In some embodiments, a variant polypeptide comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference.

In some embodiments, the protein includes a polypeptide, e.g., enzymes, structural polypeptides, signaling polypeptides, regulatory polypeptides, transport polypeptides, sensory polypeptides, motor polypeptides, defense polypeptides, storage polypeptides, transcription factors, antibodies, cytokines, hormones, catabolic polypeptides, anabolic polypeptides, proteolytic polypeptides, metabolic polypeptides, kinases, transferases, hydrolases, lyases, isomerases, ligases, enzyme modulator polypeptides, protein binding polypeptides, lipid binding polypeptides, membrane fusion polypeptides, cell differentiation polypeptides, epigenetic polypeptides, cell death polypeptides, nuclear transport polypeptides, nucleic acid binding polypeptides, reprogramming polypeptides, DNA editing polypeptides, DNA repair polypeptides, DNA recombination polypeptides, transposase polypeptides, DNA integration polypeptides, targeted endonucleases (e.g. Zinc-finger nucleases, transcription-activator-like nucleases (TALENs), cas9 and homologs thereof), recombinases, transposases, DNA polymerases, RNA polymerases, reverse transcriptases, and any combination thereof.

In some embodiments, the protein targets a protein in the cell for degradation. In some embodiments, the protein targets a protein in the cell for degradation by localizing the protein to the proteasome. In some embodiments, the protein is a wild-type protein. In some embodiments, the protein is a mutant protein.

Exemplary protein exogenous agents are described in the following subsections. In some embodiments, a lipid particle provided herein can include any of such exogenous agents. In particular embodiments, a lipid particle contains a nucleic acid encoding any of such exogenous agents.

a. Cytosolic Proteins

In some embodiments, the exogenous agent comprises a cytosolic protein, e.g., a protein that is produced in the recipient cell and localizes to the recipient cell cytoplasm. In some embodiments, the exogenous agent comprises a secreted protein, e.g., a protein that is produced and secreted by the recipient cell. In some embodiments, the exogenous agent comprises a nuclear protein, e.g., a protein that is produced in the recipient cell and is imported to the nucleus of the recipient cell. In some embodiments, the exogenous agent comprises an organellar protein (e.g., a mitochondrial protein), e.g., a protein that is produced in the recipient cell and is imported into an organelle (e.g., a mitochondrial) of the recipient cell. In some embodiments, the protein is a wild-type protein or a mutant protein. In some embodiments the protein is a fusion or chimeric protein.

b. Membrane Proteins

In some embodiments, the exogenous agent comprises a membrane protein. In some embodiments, the membrane protein comprises a chimeric antigen receptor (CAR), a T cell receptor, an integrin, an ion channel, a pore forming protein, a Toll-Like Receptor, an interleukin receptor, a cell adhesion protein, or a transport protein.

### 1) Chimeric Antigen Receptors (CARs)

In some embodiments, a payload gene described herein encodes a chimeric antigen receptor (CAR) comprising an antigen binding domain. In some embodiments, an exogenous agent described herein comprises a chimeric antigen receptor (CAR) comprising an antigen binding domain. In some embodiments, the payload is or comprises a chimeric antigen receptor (CAR) comprising an antigen binding domain. In some embodiments, the CAR is or comprises a first generation CAR comprising an antigen binding domain, a transmembrane domain, and signaling domain (e.g., one, two or three signaling domains). In some embodiments, the CAR comprises a third generation CAR comprising an antigen binding domain, a transmembrane domain, and at least three signaling domains. In some embodiments, a fourth generation CAR comprising an antigen binding domain, a transmembrane domain, three or four signaling domains, and a domain which upon successful signaling of the CAR induces expression of a cytokine gene. In some embodiments, the antigen binding domain is or comprises an scFv or Fab.

In some embodiments, the antigen binding domain targets an antigen characteristic of a cell type. In some embodiments, the antigen binding domain targets an antigen characteristic of a neoplastic cell. In some embodiments, the antigen characteristic of a neoplastic cell is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, Epidermal Growth Factor Receptors (EGFR) (including ErbB1/EGFR, ErbB2/HER2, ErbB3/HER3, and ErbB4/HER4), Fibroblast Growth Factor Receptors (FGFR) (including FGF1, FGF2, FGF3, FGF4, FGF5, FGF6, FGF7, FGF18, and FGF21) Vascular Endothelial Growth Factor Receptors (VEGFR) (including VEGF-A, VEGF-B, VEGF-C, VEGF-D, and PIGF), RET Receptor and the Eph Receptor Family (including EphA1, EphA2, EphA3, EphA4, EphA5, EphA6, EphA7, EphA8, EphA9, EphA10, EphB1, EphB2. EphB3, EphB4, and EphB6), CXCR1, CXCR2, CXCR3, CXCR4, CXCR6, CCR1, CCR2, CCR3, CCR4, CCR5, CCR6, CCR8, CFTR, CIC-1, CIC-2, CIC-4, CIC-5, CIC-7, CIC-Ka, CIC-Kb, Bestrophins, TMEM16A, GABA receptor, glycin receptor, ABC transporters, NAV1.1, NAV1.2, NAV1.3, NAV1.4, NAV1.5, NAV1.6, NAV1.7, NAV1.8, NAV1.9, sphingosin-1-phosphate receptor (SIPIR), NMDA channel, transmembrane protein, multispan transmembrane protein, T-cell receptor motifs; T-cell alpha chains; T-cell β chains; T-cell γ chains; T-cell δ chains; CCR7; CD3; CD4; CD5; CD7; CD8; CD11b; CD11c; CD16; CD19; CD20; CD21; CD22; CD25; CD28; CD34; CD35; CD40; CD45RA; CD45RO; CD52; CD56; CD62L; CD68; CD80; CD95; CD117; CD127; CD133; CD137 (4-1 BB); CD163; F4/80; IL-4Ra; Sca-1; CTLA-4; GITR; GARP; LAP; granzyme B; LFA-1; transferrin receptor; NKp46, perforin, CD4+; Th1; Th2; Th17; Th40; Th22; Th9; Tfh, Canonical Treg. FoxP3+; Tr1; Th3; Treg17; T_{RE}G; CDCP1, NT5E, EpCAM, CEA, gpA33, Mucins, TAG-72, Carbonic anhydrase IX, PSMA, Folate binding protein, Gangliosides (e.g., CD2, CD3, GM2), Lewis-γ², VEGF, VEGFR 1/2/3, αVβ3, α5β1, ErbB1/EGFR, ErbB1/HER2, ErB3, c-MET, IGF1R, EphA3, TRAIL-R1, TRAIL-R2, RANKL, FAP, Tenascin, PDL-1, BAFF, HDAC, ABL, FLT3, KIT, MET, RET, IL-1β, ALK, RANKL, mTOR, CTLA-4, IL-6, IL-6R, JAK3, BRAF, PTCH, Smoothened, PIGF, ANPEP, TIMP1, PLAUR, PTPRJ, LTBR, or ANTXR1, Folate receptor alpha (FRa), ERBB2 (Her2/neu), EphA2, IL-13Ra2, epidermal growth factor receptor (EGFR), Mesothelin, TSHR, CD19, CD123, CD22, CD30, CD171, CS-1, CLL-1, CD33, EGFRvIII, GD2, GD3, BCMA, MUC16 (CA125), LICAM, LeY, MSLN, IL13Rα1, L1-CAM, Tn Ag, prostate specific membrane antigen (PSMA), ROR1, FLT3, FAP, TAG72, CD38, CD44v6, CEA, EPCAM, B7H3, KIT, interleukin-11 receptor a (IL-11Ra), PSCA, PRSS21, VEGFR2, LewisY, CD24, platelet-derived growth factor receptor-beta (PDGFR-beta), SSEA-4, CD20, MUC1, NCAM, Prostase, PAP, ELF2M, Ephrin B2, IGF-1 receptor, CAIX, LMP2, gplOO, bcr-abl, tyrosinase, Fucosyl GM1, sLe, GM3, TGS5, HMWMAA, o-acetyl-GD2, Folate receptor beta, TEM1/CD248, TEM7R, CLDN6, GPRC5D, CXORF61, CD97, CD179a, ALK, Polysialic acid, PLACl, GloboH, NY-BR-1, UPK2, HAVCR1, ADRB3, PANX3, GPR20, LY6K, OR51E2, TARP, WT1, NY-ESO-1, LAGE-1a, MAGE-A1, legumain, HPV E6, E7, ETV6-AML, sperm protein 17, XAGE1, Tie 2, MAD-CT-1, MAD-CT-2, Major histocompatibility complex class I-related gene protein (MR1), urokinase-type plasminogen activator receptor (uPAR), Fos-related antigen 1, p53, p53 mutant, prostein, survivin, telomerase, PCTA-1/Galectin 8, MelanA/MARTI, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYPIB I, BORIS, SART3, PAX5, OY-TES1, LCK, AKAP-4, SSX2, RAGE-1, human telomerase reverse transcriptase, RU1, RU2, intestinal carboxyl esterase, mut hsp70-2, CD79a, CD79b, CD72, LAIR1, FCAR, LILRA2, CD300LF, CLEC12A, BST2, EMR2, LY75, GPC3, FCRL5, IGLL1, a neoantigen, CD133, CD15, CD184, CD24, CD56, CD26, CD29, CD44, HLA-A, HLA-B, HLA-C, (HLA-A,B,C) CD49f, CD151 CD340, CD200, tkrA, trkB, or trkC, or an antigenic fragment or antigenic portion thereof.

In some embodiments, the antigen binding domain targets an antigen characteristic of a T cell. In some embodiments, the antigen characteristic of a T cell is selected from a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, AKT1; AKT2; AKT3; ATF2; BCL10; CALM1; CD3D (CD3δ); CD3E (CD3ε); CD3G (CD3γ); CD4; CD8; CD28; CD45; CD80 (B7-1); CD86 (B7-2); CD247 (CD3ζ); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38β); MAPK12 (p38γ); MAPK13 (p38δ); MAPK14 (p38α); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments, the antigen binding domain targets an antigen characteristic of a disorder. In some embodiments, the antigen binding domain targets an antigen characteristic of an autoimmune or inflammatory disorder. In some embodiments, the autoimmune or inflammatory disorder is selected from chronic graft-vs-host disease (GVHD), lupus, arthritis, immune complex glomerulonephritis, goodpasture, uveitis, hepatitis, systemic sclerosis or scleroderma, type I diabetes, multiple sclerosis, cold agglutinin disease, Pemphigus vulgaris, Grave's disease, autoimmune hemolytic anemia, Hemophilia A, Primary Sjogren's Syndrome, thrombotic thrombocytopenia purpura, neuromyelits optica, Evan's syndrome, IgM mediated neuropathy, cyroglobulinemia, dermatomyositis, idiopathic thrombocytopenia, ankylosing spondylitis, bullous pemphigoid, acquired angioedema, chronic urticarial, antiphospholipid demyelinating polyneuropathy, and autoimmune thrombocytopenia or neutropenia or pure red cell aplasias, while exemplary non-limiting examples of alloimmune diseases include allosensitization (see, for example, Blazar et al., 2015, Am. J. Transplant, 15(4):931-41) or xenosensitization from hematopoietic or solid organ transplantation, blood transfusions, pregnancy with fetal allosensitization, neonatal alloimmune thrombocytopenia, hemolytic disease of the newborn, sensitization to foreign antigens such as can occur with replacement of inherited or acquired deficiency disorders treated with enzyme or protein replacement therapy, blood products, and gene therapy. In some embodiments, the antigen characteristic of an an autoimmune or inflammatory disorder is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor. In some embodiments, a CAR antigen binding domain binds to a ligand expressed on B cells, plasma cells, plasmablasts, CD10, CD19, CD20, CD22, CD24, CD27, CD38, CD45R, CD138, CD319, BCMA, CD28, TNF, interferon receptors, GM-CSF, ZAP-70, LFA-1, CD3 gamma, CD5 or CD2. See US 2003/0077249; WO 2017/058753; WO 2017/058850, the contents of which are herein incorporated by reference.

In some embodiments, the antigen binding domain targets an antigen characteristic of senescent cells, e.g., urokinase-type plasminogen activator receptor (uPAR). In some embodiments, the CAR may be used for treatment or prophylaxis of disorders characterized by the aberrant accumulation of senescent cells, e.g., liver and lung fibrosis, atherosclerosis, diabetes and osteoarthritis.

In some embodiments, the antigen binding domain targets an antigen characteristic of an infectious disease. In some embodiments, wherein the infectious disease is selected from HIV, hepatitis B virus, hepatitis C virus, Human herpes virus, Human herpes virus 8 (HHV-8, Kaposi sarcoma-associated herpes virus (KSHV)), Human T-lymphotrophic virus-1 (HTLV-1), Merkel cell polyomavirus (MCV), Simian virus 40 (SV40), Epstein-Barr virus, CMV, human papillomavirus. In some embodiments, the antigen characteristic of an infectious disease is selected from a cell surface receptor, an ion channel-linked receptor, an enzyme-linked receptor, a G protein-coupled receptor, receptor tyrosine kinase, tyrosine kinase associated receptor, receptor-like tyrosine phosphatase, receptor serine/threonine kinase, receptor guanylyl cyclase, histidine kinase associated receptor, HIV Env, gpl20, or CD4-induced epitope on HIV-1 Env.

In some embodiments, the CAR transmembrane domain comprises at least a transmembrane region of the alpha, beta or zeta chain of a T cell receptor, CD28, CD3 epsilon, CD45, CD4, CD5, CD8, CD9, CD16, CD22, CD33, CD37, CD64, CD80, CD86, CD134, CD137, CD154, or functional variant thereof. In some embodiments, the transmembrane domain comprises at least a transmembrane region(s) of CD8a, CD8B, 4-1BB/CD137, CD28, CD34, CD4, FcϵRIγ, CD16, OX40/CD134, CD35, CD38, CD3Y, CD38, TCRα, TCRβ, TCRζ, CD32, CD64, CD64, CD45, CD5, CD9, CD22, CD37, CD80, CD86, CD40, CD40L/CD154, VEGFR2, FAS, and FGFR2B, or functional variant thereof.

In some embodiments, the CAR comprises at least one signaling domain selected from one or more of B7-1/CD80; B7-2/CD86; B7-H1/PD-L1; B7-H2; B7-H3; B7-H4; B7-H6; B7-H7; BTLA/CD272; CD28; CTLA-4; Gi24/VISTA/B7-H5; ICOS/CD278; PD-1; PD-L2/B7-DC; PDCD6); 4-1BB/TNFSF9/CD137; 4-1BB Ligand/TNFSF9; BAFF/BLyS/TNFSF13B; BAFF R/TNFRSF13C; CD27/TNFRSF7; CD27 Ligand/TNFSF7; CD30/TNFRSF8; CD30 Ligand/TNFSF8; CD40/TNFRSF5; CD40/TNFSF5; CD40 Ligand/TNFSF5; DR3/TNFRSF25; GITR/TNFRSF18; GITR Ligand/TNFSF18; HVEM/TNFRSF14; LIGHT/TNFSF14; Lymphotoxin-alpha/TNF-beta; OX40/TNFRSF4; OX40 Ligand/TNFSF4; RELT/TNFRSF19L; TACI/TNFRSF13B; TL1A/TNFSF15; TNF-alpha; TNF RII/TNFRSF1B); 2B4/CD244/SLAMF4; BLAME/SLAMF8; CD2; CD2F-10/SLAMF9; CD48/SLAMF2; CD58/LFA-3; CD84/SLAMF5; CD229/SLAMF3; CRACC/SLAMF7; NTB-A/SLAMF6; SLAM/CD150); CD2; CD7; CD53; CD82/Kai-1; CD90/Thy1; CD96; CD160; CD200; CD300a/LMIR1; HLA Class I; HLA-DR; Ikaros; Integrin alpha 4/CD49d; Integrin alpha 4 beta 1; Integrin alpha 4 beta 7/LPAM-1; LAG-3; TCL1A; TCL1B; CRTAM; DAP12; Dectin-1/CLEC7A; DPPIV/CD26; EphB6; TIM-1/KIM-1/HAVCR; TIM-4; TSLP; TSLP R; lymphocyte function associated antigen-1 (LFA-1); NKG2C, a CD3 zeta domain, an immunoreceptor tyrosine-based activation motif (ITAM), CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83, or functional fragment thereof.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In some embodiments the exogenous agent is or comprises a CAR, e.g., a first generation CAR or a nucleic acid encoding a first generation CAR. In some embodiments, a first generation CAR comprises an antigen binding domain, a transmembrane domain, and signaling domain. In some embodiments a signaling domain mediates downstream signaling during T cell activation.

In some embodiments the exogenous agent is or comprises a second generation CAR or a nucleic acid encoding a second generation CAR. In some embodiments a second generation CAR comprises an antigen binding domain, a transmembrane domain, and two signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments the exogenous agent is or comprises a third generation CAR or a nucleic acid encoding a third generation CAR. In some embodiments, a third generation CAR comprises an antigen binding domain, a transmembrane domain, and at least three signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation. In some embodiments, a third generation CAR comprises at least two costimulatory domains. In some embodiments, the at least two costimulatory domains are not the same.

In some embodiments the exogenous is or comprises a fourth generation CAR or a nucleic acid encoding a fourth generation CAR. In some embodiments a fourth generation CAR comprises an antigen binding domain, a transmembrane domain, and at least two, three, or four signaling domains. In some embodiments a signaling domain mediates downstream signaling during T cell activation. In some embodiments a signaling domain is a costimulatory domain. In some embodiments, a costimulatory domain enhances cytokine production, CAR T cell proliferation, and or CAR T cell persistence during T cell activation.

In some embodiments, a first, second, third, or fourth generation CAR further comprises a domain which upon successful signaling of the CAR induces expression of a cytokine gene. In some embodiments, a cytokine gene is endogenous or exogenous to a target cell comprising a CAR which comprises a domain which upon successful signaling of the CAR induces expression of a cytokine gene. In some embodiments a cytokine gene encodes a pro-inflammatory cytokine. In some embodiments a cytokine gene encodes IL-1, IL-2, IL-9, IL-12, IL-18, TNF, or IFN-gamma, or functional fragment thereof. In some embodiments a domain which upon successful signaling of the CAR induces expression of a cytokine gene is or comprises a transcription factor or functional domain or fragment thereof. In some embodiments a domain which upon successful signaling of the CAR induces expression of a cytokine gene is or comprises a transcription factor or functional domain or fragment thereof. In some embodiments a transcription factor or functional domain or fragment thereof is or comprises a nuclear factor of activated T cells (NFAT), an NF-kB, or functional domain or fragment thereof. See, e.g., Zhang. C. et al., Engineering CAR-T cells. Biomarker Research. 5:22 (2017); WO 2016126608; Sha, H. et al. Chimaeric antigen receptor T-cell therapy for tumour immunotherapy. Bioscience Reports Jan. 27, 2017, 37 (1).

In some embodiments, a CAR antigen binding domain is or comprises an antibody or antigen-binding portion thereof. In some embodiments, a CAR antigen binding domain is or comprises an scFv or Fab. In some embodiments a CAR antigen binding domain comprises an scFv or Fab fragment of a T-cell alpha chain antibody; T-cell β chain antibody; T-cell γ chain antibody; T-cell δ chain antibody; CCR7 antibody; CD3 antibody; CD4 antibody; CD5 antibody; CD7 antibody; CD8 antibody; CD11b antibody; CD11c antibody; CD16 antibody; CD19 antibody; CD20 antibody; CD21 antibody; CD22 antibody; CD25 antibody; CD28 antibody; CD34 antibody; CD35 antibody; CD40 antibody; CD45RA antibody; CD45RO antibody; CD52 antibody; CD56 antibody; CD62L antibody; CD68 antibody; CD80 antibody; CD95 antibody; CD117 antibody; CD127 antibody; CD133 antibody; CD137 (4-1 BB) antibody; CD163 antibody; F4/80 antibody; IL-4Ra antibody; Sca-1 antibody; CTLA-4 antibody; GITR antibody GARP antibody; LAP antibody; granzyme B antibody; LFA-1 antibody; MR1 antibody; uPAR antibody; or transferrin receptor antibody.

In some embodiments, an antigen binding domain binds to a cell surface antigen of a cell. In some embodiments, a cell surface antigen is characteristic of one type of cell. In some embodiments, a cell surface antigen is characteristic of more than one type of cell.

In some embodiments a CAR antigen binding domain binds a cell surface antigen characteristic of a T cell. In some embodiments, an antigen characteristic of a T cell may be a cell surface receptor, a membrane transport protein (e.g., an active or passive transport protein such as, for example, an ion channel protein, a pore-forming protein, etc.), a transmembrane receptor, a membrane enzyme, and/or a cell adhesion protein characteristic of a T cell. In some embodiments, an antigen characteristic of a T cell may be a G protein-coupled receptor, receptor tyrosine kinase, tyrosine kinase associated receptor, receptor-like tyrosine phosphatase, receptor serine/threonine kinase, receptor guanylyl cyclase, or histidine kinase associated receptor.

In some embodiments, an antigen characteristic of a T cell may be a T cell receptor. In some embodiments, a T cell receptor may be AKT1; AKT2; AKT3; ATF2; BCL10; CALM1; CD3D (CD38); CD3E (CD38); CD3G (CD3Y); CD4; CD8; CD28; CD45; CD80 (B7-1); CD86 (B7-2); CD247 (CD3° C.); CTLA4 (CD152); ELK1; ERK1 (MAPK3); ERK2; FOS; FYN; GRAP2 (GADS); GRB2; HLA-DRA; HLA-DRB1; HLA-DRB3; HLA-DRB4; HLA-DRB5; HRAS; IKBKA (CHUK); IKBKB; IKBKE; IKBKG (NEMO); IL2; ITPR1; ITK; JUN; KRAS2; LAT; LCK; MAP2K1 (MEK1); MAP2K2 (MEK2); MAP2K3 (MKK3); MAP2K4 (MKK4); MAP2K6 (MKK6); MAP2K7 (MKK7); MAP3K1 (MEKK1); MAP3K3; MAP3K4; MAP3K5; MAP3K8; MAP3K14 (NIK); MAPK8 (JNK1); MAPK9 (JNK2); MAPK10 (JNK3); MAPK11 (p38B); MAPK12 (p38γ); MAPK13 (p388); MAPK14 (p38a); NCK; NFAT1; NFAT2; NFKB1; NFKB2; NFKBIA; NRAS; PAK1; PAK2; PAK3; PAK4; PIK3C2B; PIK3C3 (VPS34); PIK3CA; PIK3CB; PIK3CD; PIK3R1; PKCA; PKCB; PKCM; PKCQ; PLCY1; PRF1 (Perforin); PTEN; RAC1; RAF1; RELA; SDF1; SHP2; SLP76; SOS; SRC; TBK1; TCRA; TEC; TRAF6; VAV1; VAV2; or ZAP70.

In some embodiments a CAR comprises a signaling domain which is a costimulatory domain. In some embodiments a CAR comprises a second costimulatory domain. In some embodiments a CAR comprises at least two costimulatory domains. In some embodiments a CAR comprises at least three costimulatory domains. In some embodiments a CAR comprises a costimulatory domain selected from one or more of CD27, CD28, 4-1BB, CD134/OX40, CD30, CD40, PD-1, ICOS, lymphocyte function-associated antigen-1 (LFA-1), CD2, CD7, LIGHT, NKG2C, B7-H3, a ligand that specifically binds with CD83.

In some embodiments, the CAR comprises a CD3 zeta domain or an immunoreceptor tyrosine-based activation motif (ITAM), or functional variant thereof. In some embodiments, the CAR comprises (i) a CD3 zeta domain, or an immunoreceptor tyrosine-based activation motif (ITAM), or functional variant thereof; and (ii) a CD28 domain, or a 4-1BB domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; and (iii) a 4-1BB domain, or a CD134 domain, or functional variant thereof. In some embodiments, the CAR comprises (i) a CD3 zeta domain, or an immunoreceptor tyrosine-based activation motif (ITAM), or functional variant thereof; (ii) a CD28 domain, or a 4-1BB domain, or functional variant thereof, and/or (iii) a 4-1BB domain, or a CD134 domain, or functional variant thereof. In some embodiments, the CAR comprises a (i) a CD3 zeta domain, or an immunoreceptor tyrosine-based activation motif (ITAM), or functional variant thereof; (ii) a CD28 domain or functional variant thereof; (iii) a 4-1BB domain, or a CD134 domain, or functional variant thereof; and (iv) a cytokine or costimulatory ligand transgene.

In certain embodiments, the intracellular signaling domain comprises a CD28 transmembrane and signaling domain linked to a CD3 (e.g., CD3-zeta) intracellular domain. In some embodiments, the intracellular signaling domain comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some embodiments the intracellular signaling domain includes intracellular components of a 4-1BB signaling domain and a CD3-zeta signaling domain. In some embodiments, the intracellular signaling domain includes intracellular components of a CD28 signaling domain and a CD3zeta signaling domain.

In some embodiments, the CAR comprises an extracellular antigen binding domain (e.g., antibody or antibody fragment, such as an scFv) that binds to an antigen (e.g. tumor antigen), a spacer (e.g. containing a hinge domain, such as any as described herein), a transmembrane domain (e.g. any as described herein), and an intracellular signaling domain (e.g. any intracellular signaling domain, such as a primary signaling domain or costimulatory signaling domain as described herein). In some embodiments, the intracellular signaling domain is or includes a primary cytoplasmic signaling domain. In some embodiments, the intracellular signaling domain additionally includes an intracellular signaling domain of a costimulatory molecule (e.g., a costimulatory domain). Examples of exemplary components of a CAR are described in Table 2. In provided aspects, the sequences of each component in a CAR can include any combination listed in Table 2.

TABLE 2
CAR components and Exemplary Sequences

| | | SEQ |
|---|---|---|
| | | ID |
| Component | Sequence | NO |
| Extracellular | | |
| binding | | |
| domain | | |
| Anti-CD19 | DIQMTQTTSSLSASLGDRVTISCRASQDISKY | 61 |
| scFv | LNWYQQKPDGTVKLLIYHTSRLHSGVPSRFS | |
| (FMC63) | GSGSGTDYSLTISNLEQEDIATYFCQQGNTLP | |
| | YTFGGGTKLEITGSTSGSGKPGSGEGSTKGE | |
| | VKLQESGPGLVAPSQSLSVTCTVSGVSLPDY | |
| | GVSWIRQPPRKGLEWLGVIWGSETTYYNSA | |
| | LKSRLTIIKDNSKSQVFLKMNSLQTDDTAIYY | |
| | CAKHYYYGGSYAMDYWGQGTSVTVSS | |
| Anti-CD19 | DIQMTQTTSSLSASLGDRVTISCRASQDISKY | 62 |
| scFv | LNWYQQKPDGTVKLLIYHTSRLHSGVPSRFS | |
| (FMC63) | GSGSGTDYSLTISNLEQEDIATYFCQQGNTLP | |
| | YTFGGGTKLEITGGGGSGGGGSGGGGSEVK | |
| | LQESGPGLVAPSQSLSVTCTVSGVSLPDYGV | |
| | SWIRQPPRKGLEWLGVIWGSETTYYNSALKS | |
| | RLTIIKDNSKSQVFLKMNSLQTDDTAIYYCA | |
| | KHYYYGGSYAMDYWGQGTSVTVSS | |
| Spacer | | |
| (e.g. hinge) | | |
| IgG4 Hinge | ESKYGPPCPPCP | 63 |
| CD8 Hinge | TTTPAPRPPTPAPTIASQPLSLRPE | 64 |
| CD28 | IEVMYPPPYLDNEKSNGTIIHVKGKHLCPSPL | 65 |
| | FPGPSKP | |
| Transmembrane | | |
| CD8 | ACRPAAGGAVHTRGLDFACDIYIWAPLAGT | 66 |
| | CGVLLLSLVITLYC | |
| CD28 | FWVLVVVGGVLACYSLLVTVAFIIFWV | 67 |
| CD28 | FWVLVVVGGVLACYSLLVTVAFIIFWV | 68 |
| Costimulatory | | |
| domain | | |
| CD28 | RSKRSRLLHSDYMNMTPRRPGPTRKHYQPY | 69 |
| | APPRDFAAYRS | |
| 4-1BB | KRGRKKLLYIFKQPFMRPVQTTQEEDGCSCR | 70 |
| | FPEEEEGGCEL | |
| Primary | | |
| Signaling | | |
| Domain | | |
| CD3zeta | RVKFSRSADAPAYQQGQNQLYNELNLGRRE | 71 |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |
| CD3zeta | RVKFSRSADAPAYKQGQNQLYNELNLGRRE | 72 |
| | EYDVLDKRRGRDPEMGGKPRRKNPQEGLY | |
| | NELQKDKMAEAYSEIGMKGERRRGKGHDG | |
| | LYQGLSTATKDTYDALHMQALPPR | |

In some embodiments, the CAR further comprises one or more spacers, e.g., wherein the spacer is a first spacer between the antigen binding domain and the transmembrane domain. In some embodiments, the first spacer includes at least a portion of an immunoglobulin constant region or variant or modified version thereof. In some embodiments, the spacer is a second spacer between the transmembrane domain and a signaling domain. In some embodiments, the second spacer is an oligopeptide, e.g., wherein the oligopeptide comprises glycine-serine doublets.

In addition to the CARs described herein, various chimeric antigen receptors and nucleotide sequences encoding the same are known in the art and would be suitable for fusosomal delivery and reprogramming of target cells in vivo and in vitro as described herein. See, e.g., WO2013040557; WO2012079000; WO2016030414; Smith T, et al., Nature Nanotechnology. 2017. DOI: 10.1038/NNANO.2017.57, the disclosures of which are herein incorporated by reference.

In some embodiments a lipid particle comprising a CAR or a nucleic acid encoding a CAR (e.g., a DNA, a gDNA, a cDNA, an RNA, a pre-MRNA, an mRNA, an miRNA, an siRNA, etc.) is delivered to a target cell. In some embodiments the target cell is an effector cell, e.g., a cell of the immune system that expresses one or more Fc receptors and mediates one or more effector functions. In some embodiments, a target cell may include, but may not be limited to, one or more of a monocyte, macrophage, neutrophil, dendritic cell, eosinophil, mast cell, platelet, large granular lymphocyte, Langerhans' cell, natural killer (NK) cell, T lymphocyte (e.g., T cell), a Gamma delta T cell, B lymphocyte (e.g., B cell) and may be from any organism including but not limited to humans, mice, rats, rabbits, and monkeys.

3. Gene Editing Agents (e.g. Nuclease Enzymes)

In some embodiments, the exogenous agent is associated with a gene editing technology. Any of a variety of agents associated with gene editing technologies can be included as the exogenous agent, such as for delivery of gene editing machinery to a cell. In some embodiments, the gene editing technology can include systems involving nucleases, integrases, transposases, recombinases. In some embodiments, the gene editing technologies can be used for knock-out or knock-down of genes. In some embodiments, the gene-editing technologies can be used for knock-in or integration of DNA into a region of the genome. In some embodiments, the exogenous agent mediates double-strand breaks (DSB), including in connection with non-homologous end-joining (NHEJ) or homology-directed repair (HDR). In some embodiments, the exogenous agent does not mediate DSB. In some embodiments, the exoneous agent can be used for DNA-based editing or prime-editing. In some embodiments, the exogenous agent can be used for Programmable Addition via Site-specific Targeting Elements (PASTE).

In some embodiments, the exogenous agent is a nuclease for use in gene editing methods. In some embodiments, the nuclease is a zinc-finger nucleases (ZFNs), transcription-activator like effector nucleases (TALENs), or a CRISPR-associated protein-nuclease (Cas). In some embodiments, the Cas is Cas9 from Streptococcus pyogenes. In some embodiments, the Cas is a Cas12a (also known as cpf1) from a Prevotella or Francisella bacteria. In some embodiments, the Cas is a Cas12b from a Bacillus, optionally Bacillus hisashii.

In some embodiments, delivery of the nuclease is by a provided vector encoding the nuclease (e.g. Cas).

In some embodiments, the provided viral vector particles contain a nuclease protein and the nuclease protein is directly delivered to a target cell. Methods of delivering a nucleobase protein include those as described, for example, in Cai et al. Elife, 2014, 3:e01911 and International patent publication No. WO2017068077. For instance, provided viral vector particles comprise one or more Cas protein(s), such as Cas9. In some embodiments, the nuclease protein (e.g. Cas, such as Cas 9) is engineered as a chimeric nuclease protein with a viral structural protein (e.g. GAG) for packaging into the viral vector particle (e.g. lentiviral vector particle). For instance, a chimeric Cas9-protein fusion with the structural GAG protein can be packaged inside a lentiviral vector particle. In some embodiments, the fusion protein is a cleavable fusion protein between (i) a viral structural protein (e.g. GAG) and (ii) a nuclease protein (e.g. Cas protein, such as Cas 9).

In some embodiments, the Cas is wild-type Cas9, which can site-specifically cleave double-stranded DNA, resulting in the activation of the double-strand break (DSB) repair machinery. DSBs can be repaired by the cellular Non-Homologous End Joining (NHEJ) pathway (Overballe-Petersen et al., 2013, Proc Natl Acad Sci USA, Vol. 110: 19860-19865), resulting in insertions and/or deletions (indels) which disrupt the targeted locus. Alternatively, if a donor template with homology to the targeted locus is supplied, the DSB may be repaired by the homology-directed repair (HDR) pathway allowing for precise replacement mutations to be made (Overballe-Petersen et al., 2013, Proc Natl Acad Sci USA, Vol. 110: 19860-19865; Gong et al., 2005, Nat. Struct Mol Biol, Vol. 12: 304-312). In some embodiments, the Cas is mutant form, known as Cas9 D10A, with only nickase activity. This means that Cas9D10A cleaves only one DNA strand, and does not activate NHEJ. Instead, when provided with a homologous repair template, DNA repairs are conducted via the high-fidelity HDR pathway only, resulting in reduced indel mutations (Cong et al., 2013, Science, Vol. 339: 819-823; Jinek et al., 2012, Science, Vol. 337: 816-821; Qi et al., 2013 Cell, Vol. 152: 1173-1183). Cas9D10A is even more appealing in terms of target specificity when loci are targeted by paired Cas9 complexes designed to generate adjacent DNA nicks (Ran et al., 2013, Cell, Vol. 154: 1380-1389). In some embodiments, the Cas is a nuclease-deficient Cas9 (Qi et al., 2013 Cell, Vol. 152: 1173-1183). For instance, mutations H840A in the HNH domain and D10A in the RuvC domain inactivate cleavage activity, but do not prevent DNA binding. Therefore, this variant can be used to target in a sequence-specific manner any region of the genome without cleavage. Instead, by fusing with various effector domains, dCas9 can be used either as a gene silencing or activation tools. Furthermore, it can be used as a visualization tool by coupling the guide RNA or the Cas9 protein to a fluorophore or a fluorescent protein.

In particular embodiments, the nuclease is a Cas nuclease, such as Cas9. In some embodiments, delivery of the CRISPR/Cas can be used to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. In some embodiments, a dCas9 version of the CRISPR/Cas9 system can be used to target protein domains for transcriptional regulation, epigenetic modification, and microscopic visualization of specific genome loci.

In some embodiments, the provided virus particles (e.g. lentiviral particles) containing a Cas nuclease (e.g. Cas9) further comprise, or is further complexed with, one or more CRISPR-Cas system guide RNA(s) for targeting a desired target gene. In some embodiments, the CRISPR guide RNAs are efficiently encapsulated in the CAS-containing viral particles. In some embodiments, the provided virus particles (e.g. lentiviral particles) further comprises, or is further complexed with a targeting nucleic acid.

In some embodiments, the exogenous agent is one for use in target-primed reverse transcription (TPRT) or “prime editing”. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, the exogenous agent is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA

In some embodiments, the exogenous agent is or encodes for a primer editor that is a reverse transcriptase, or any DNA polymerase known in the art. Thus, in one aspect, the prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (https://doi.org/10.1038/s41586-019-1711-4), or in PCT publication Nos. WO2020191248, WO2021226558, or WO2022067130, which are hereby incorporated in their entirety.

In some embodiments, the exogenous agent is for use in Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi: https://doi.org/10.1101/2021.11.01.466786), PASTE does not generate double stranded breaks, but allowed for integration of sequences as large as ˜36 kb. In some embodiments, the serine integrase can be any known in the art. In some embodiments, the serine integrase has sufficient orthogonality such that PASTE can be use for multiplexed gene integration, simultaneously integrating at least two different genes at at least two genomic loci. In some embodiments, PASTE has editing efficiencies comparable to or better than those of homology directed repair or non-homologous end joining based integration, with activity in nondividing cells and fewer detectable off-target events. In some embodiments, the exogenous agent is associate with base editing. Base editors (BEs) are typically fusions of a Cas (“CRISPR-associated”) domaindomain and a nucleobase modification domaindomain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some cases, base editors may also include proteins or domains that alter cellular DNA repair processes to increase the efficiency and/or stability of the resulting single-nucleotide change.

In some aspects, currently available base editors include cytidine base editors (e.g., BE4) that convert target C⋅G to T⋅A and adenine base editors (e.g., ABE7.10) that convert target A⋅T to G⋅C. In some aspects, Cas9-targeted deamination was first demonstrated in connection with a Base Editor (BE) system designed to induce base changes without introducing double-stranded DNA breaks. Further Rat deaminase APOBEC1 (rAPOBEC1) fused to deactivated Cas9 (dCas9) was used to successfully convert cytidines to thymidines upstream of the PAM of the sgRNA. In some aspects, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T:A during DNA replication.

In some embodiments, the exogenous agent is or encodes a base editor (e.g., a nucleobase editor). In some embodiments, the exogenous agent is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, the base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, the base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, the base editor is a adenine-to-thymine or “ATBE” (or thymine-to-adenine or “TABE”) transversion base editors. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, the exogenous agent is or encodes one or more polypeptides having an activity selected from the group consisting of: nuclease activity (e.g., programmable nuclease activity); nickase activity (e.g., programmable nickase activity); homing activity (e.g., programmable DNA binding activity); nucleic acid polymerase activity (e.g., DNA polymerase or RNA polymerase activity); integrase activity; recombinase activity; or base editing activity (e.g., cytidine deaminase or adenosine deaminase activity).

### 4. Small Molecules

In some embodiments, the exogenous agent includes a small molecule, e.g., ions (e.g. Ca²⁺, Cl—, Fe²⁺), carbohydrates, lipids, reactive oxygen species, reactive nitrogen species, isoprenoids, signaling molecules, heme, polypeptide cofactors, electron accepting compounds, electron donating compounds, metabolites, ligands, and any combination thereof. In some embodiments the small molecule is a pharmaceutical that interacts with a target in the cell. In some embodiments the small molecule targets a protein in the cell for degradation. In some embodiments the small molecule targets a protein in the cell for degradation by localizing the protein to the proteasome. In some embodiments that small molecule is a proteolysis targeting chimera molecule (PROTAC).

In some embodiments, the exogenous agent includes a mixture of proteins, nucleic acids, or metabolites, e.g., multiple polypeptides, multiple nucleic acids, multiple small molecules; combinations of nucleic acids, polypeptides, and small molecules; ribonucleoprotein complexes (e.g. Cas9-gRNA complex); multiple transcription factors, multiple epigenetic factors, reprogramming factors (e.g. Oct4, Sox2, cMyc, and Klf4); multiple regulatory RNAs; and any combination thereof.

### D. Exemplary Features

In some embodiments, the truncated BaEV envelope glycoprotein has increased or greater expression on the surface of a particle compared to a reference particle but containing a BaEV that lacks all amino acid residues of the R peptide (e.g. BaEV set forth in SEQ ID NO:24). In some embodiments, the expression is increased by at or greater than 5%, 10%, 20%, 30%, 40%, 50%, 60%, 70%, 80%, 90%, 100%, 125%, 150%, 200%, 300%, 400%, 500% or more compared to a reference particle but containing a BaEV that lacks all amino acid residues of the R peptide (e.g. BaEV set forth in SEQ ID NO:24). In some embodiments, the expression is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, preferably at or about or greater than 10-fold or more compared to a reference particle but containing a BaEV that lacks all amino acid residues of the R peptide (e.g. BaEV set forth in SEQ ID NO:24). In some embodiments, expression can be assayed in vitro using flow cytometry, e.g. FACs. In some embodiments, expression can be depicted as the number or density of the BaEV envelope glycoprotein, e.g. truncated BaEV envelope glycoprotein, on the surface of a lipid particle. In some embodiments, expression can be depicted as the mean fluorescent intensity (MFI) of surface expression of the truncated BaEV envelope glycoprotein, e.g. truncated BaEV envelope glycoprotein, on the surface of a lipid particle. In some embodiments, expression can be depicted as the percent of lipid particle (e.g. lentiviral vectors) in a population that are surface positive for the BaEV envelope glycoprotein, e.g. truncated BaEV envelope glycoprotein.

In some embodiments, in a population of lipid particles (e.g. lentiviral vectors) greater than at or about 50% of the lipid particles are surface positive for the truncated BaEV envelope glycoprotein. For example, in a population of provided lipid particles (e.g. lentiviral vectors) greater than at or about 55%, greater than at or about 60%, greater than at or about 65%, greater than at or about 70%, greater than at or about 75% of the cells in the population are surface positive for the truncated BaEV envelope glycoprotein.

In some embodiments, titer of the lipid particles following introduction into target cells, such as by transduction (e.g. transduced cells), is increased compared to titer into the same target cells of reference lipid particles (e.g. reference lentiviral vector) that incorporate a BaEV that lacks all amino acid residues of the R peptide (e.g. BaEV set forth in SEQ ID NO:24). In some examples, the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, compared to the titer of a reference lipid particle (e.g. reference lentiviral vector), e.g. a reference lipid particle containing a BaEV that lacks all amino acid residues of the R peptide (e.g. BaEV set forth in SEQ ID NO:24). In some of any embodiments, the titer in target cells following transduction is at or greater than 1×10⁶ transduction units (TU)/mL, at or greater than 2×10⁶ TU/mL, at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, or at or greater than 1×10⁷ TU/mL.

In particular embodiments, the truncated BaEV envelope glycoprotein is present on the surface of the particle at a density of at least about (0.001, 0.002, 0.005, 0.01, 0.02, 0.05, 0.1, 0.2 or 0.5) truncated BaEV envelope glycoproteins/nm².

In some embodiments, the provided lipid particles preferentially target a target cell compared to a non-target cell. In some embodiments, the provided lipid particles contain an exogenous agent inside the lumen or cavity and such lipid particles exhibit preferential delivery of the exogenous agent to a target cell compared to a non-target cell.

As used herein, a “target cell” refers to a cell of a type that is specifically targeted by a BaEV envelope glycoprotein-containing lipid particle. In embodiments, a target cell is a hematopoietic cell, such as a cell that is surface positive for CD34 (CD34+ cell).

As used herein a “non-target cell” refers to a cell of a type to which targeting of a lipid particle, e.g. for delivery of an exogenous agent, is not desired. In some embodiments, a non-target cell is a non-hematopoietic cells. In some embodiments, a non-target cells is a cell that is surface negative for CD34 (CD34− cell).

In some embodiments, lipid particles containing a truncated BaEV envelope glycoprotein, such as a BaEV glycoprotein pseudotyped lentiviral particle, are capable of targeting a cell ex vivo. In some embodiments, lipid particles containing a truncated BaEV envelope glycoprotein, such as a BaEV glycoprotein pseudotyped lentiviral particle, are capable of targeting a cell in vivo. In some embodiments, lipid particles containing a truncated BaEV envelope glycoprotein, such as a BaEV glycoprotein pseudotyped lentiviral particle, are capable of targeting a cell in vivo after mobilization.

In some embodiments, the truncated BaEV envelope glycoproteins exhibit similar tropism and cell targeting as VSV-G. Thus, in some embodiments, lipid particles containing a truncated Baboon Endogenous Retrovirus (BaEV) envelope glycoprotein, such as aBaEV glycoprotein pseudotyped lentiviral particle, can be used tin place of VSV-G.

In some embodiments, the target cell is positive for cell surface expression of ASCT1 and/or ASCT2.

In some embodiments, the target cell is a hematopoietic lineage cell. Reference to a “hematopoietic cell” includes blood cells, both from the myeloid and the lymphoid lineage. In particular, the term “hematopoietic cell” includes both undifferentiated or poorly differentiated cells such as hematopoietic stem cells and progenitor cells, and differentiated cells such as T lymphocytes, B lymphocytes or dendritic cells. In some embodiments, the hematopoietic cell is a hematopoietic stem cells, CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, and thymocytes.

As known from the skilled person, many hematopoietic cells are produced from bone marrow hematopoietic stem cells.

In some embodiments, a hematopoietic cell is a hematopoietic stem cell (HSC), which are cells able to replenish all blood cell types and to self-renew. Hematopoietic stem cells may be in particular defined as cells that keep the levels of myeloid, T and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the hematopoietic cells is a “CD34+ progenitor cell,” which is a heterogeneous cell population that includes a subpopulation of HSCs, pluripotent stem cells and cells in the early stages of lineage commitment. CD34+ progenitor cells continuously migrate to and from the bone marrow in normal adult animals. They can differentiate to produce all hematopoietic cell lineages found in the circulation. In some embodiments, the hematopoietic cell is a very early progenitor CD34+ cell which is a subgroup of CD34+ progenitor cells enriched from HSCs.

In some embodiments, the hematopoietic cells includes “peripheral blood CD34+ cell,” which are CD34+ cells present in the blood.

In some embodiments, the hematopoietic cell is a B cell CD19+ progenitor, which is a population of B-lineage cells that express cell surface CD10, CD34 and CD19.

In some embodiments, the hematopoietic cells is a myeloid progenitor CD13+ cells, which is a population of myeloid lineage cells that express cell surface CD34 and CD34, and in some cases, also CD33.

In some embodiments, the cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells. In some embodiments, the cell is selected from monocytes, macrophages, neutrophils, basophils, eosinophils, erythrocytes, megakaryocytes and platelets. In some embodiments, the cell is selected from T cells, B cells, natural killer (NK) cells and innate lymphoid cells.

In some embodiment, the hematopoietic cell is a T cell. In some embodiments, the T cell is a naïve T cell. In some embodiments, the T cell is a memory T cell.

In some embodiments, the hematopoietic cell is a B cell. In some embodiments, the targeted cell is a resting B cell, such as a naive or a memory B cell. In some embodiments, the targeted cell is a cancer B cell, such as a B-cell chronic lymphocytic leukemia (BCLL) cell or a marginal zone lymphoma (MZL) B cell.

In some embodiments, the targeted cell is a thymocyte. In some embodiments, the targeted cell is a natural killer (NK) cell. In some embodiments, the thymocyte expresses CD4 or CD8. In some embodiments, the thymocyte does not express CD4 or CD8. In some embodiments, the natural killer (NK) cell is a cell that expresses CD56.

### IV. Pharmaceutical Compositions and Methods of Manufacture

Also provided are compositions containing the lipid particles herein containing a truncated BaEV envelope glycoprotein or polynucleotides encoding the truncated BaEV envelope glycoproteins, including pharmaceutical compositions and formulations. The pharmaceutical compositions can include any of the described truncated BaEV-containing lipid particles.

The present disclosure also provides, in some aspects, a pharmaceutical composition comprising the composition described herein and pharmaceutically acceptable carrier.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular lipid particle and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

In some embodiments, the lipid particle meets a pharmaceutical or good manufacturing practices (GMP) standard. In some embodiments, the lipid particle is made according to good manufacturing practices (GMP). In some embodiments, the lipid particle has a pathogen level below a predetermined reference value, e.g., is substantially free of pathogens. In some embodiments, the lipid particle has a contaminant level below a predetermined reference value, e.g., is substantially free of contaminants. In some embodiments, the lipid particle has low immunogenicity.

In some embodiments, formulations of the pharmaceutical compositions described herein may be prepared by any method known or hereafter developed in the art of pharmacology. In some embodiments, preparatory methods include the step of bringing the active ingredient into association with a carrier or one or more other accessory ingredients, and then, if necessary or desirable, shaping or packaging the product into a desired single- or multi-dose unit.

In some embodiments, a “unit dose” is a discrete amount of the pharmaceutical composition comprising a predetermined amount of the active ingredient. In some embodiments, the amount of the active ingredient is generally equal to the dosage of the active ingredient that would be administered to a subject or a convenient fraction of such a dosage such as, for example, one-half or one-third of such a dosage. In some embodiments, the unit dosage form may be for a single daily dose or one of multiple daily doses (e.g., about 1 to 4 or more times per day). In some embodiments, when multiple daily doses are used, the unit dosage form may be the same or different for each dose.

In some embodiments, the lipid particle containing the truncated BaEV envelope glycoproteins is a viral vector or virus-like particle (e.g., Section II). In some embodiments, the compositions provided herein can be formulated in dosage units of genome copies (GC). Suitable method for determining GC have been described and include, e.g., qPCR or digital droplet PCR (ddPCR) as described in, e.g., M. Lock et al, Hu Gene Therapy Methods, Hum Gene Ther Methods 25(2): 115-25. 2014, which is incorporated herein by reference. In some embodiments, the dosage of administration of a viral vector or virus-like particle is from about 10⁴ to about 10¹⁰ GC units, inclusive. In some embodiments, the dosage of administration of a viral vector or virus-like particle is from about 10⁹ to about 10¹⁵ GC units, inclusive. In some embodiments, the dosage of administration of a viral vector or virus-like particle is from about 10⁵ to about 10⁹ GC units, inclusive. In some embodiments, the dosage of administration of a viral vector or virus-like particle is from about 10⁶ to about 10⁹ GC units, inclusive. In some embodiments, the dosage of administration of a viral vector or virus-like particle is from about 10¹² to about 10¹⁴ GC units, inclusive. In some embodiments, the dosage of administration is 1.0×10⁹ GC units, 5.0×10⁹ GC units, 1.0×10¹⁰ GC units, 5.0×10¹⁰ GC units, 1.0×10¹¹ GC units, 5.0×10¹¹ GC units, 1.0×10¹² GC units, 5.0×10¹² GC units, or 1.0×10¹³ GC units, 5.0×10¹³ GC units, 1.0×10¹⁴ GC units, 5.0×10¹⁴ GC units, or 1.0×10¹⁵ GC units.

In some embodiments, the dosage of administration of a viral vector or virus-like particle is from about 10⁴ to about 10¹⁰ infectious units, inclusive. In some embodiments, the dosage of administration of a viral vector or virus-like particle is from about 10⁹ to about 10¹⁵ infectious units, inclusive In some embodiments, the dosage of administration of a viral vector or virus-like particle is from about 10⁵ to about 10⁹ infectious units. In some embodiments, the dosage of administration of a viral vector or virus-like particle is from about 10⁶ to about 10⁹ infectious units. In some embodiments, the dosage of administration of a viral vector or virus-like particle is from about 10¹² to about 10¹⁴ infectious units, inclusive. In some embodiments, the dosage of administration is 1.0×10⁹ infectious units, 5.0×10⁹ infectious units, 1.0×10¹⁰ infectious units, 5.0×10¹⁰ infectious units, 1.0×10¹¹ infectious units, 5.0×10¹¹ infectious units, 1.0×10¹² infectious units, 5.0×10¹² infectious units, or 1.0×10¹³ infectious units, 5.0×10¹³ infectious units, 1.0×10¹⁴ infectious units, 5.0×10¹⁴ infectious units, or 1.0×10¹⁵ infectious units. The techniques available for quantifying infectious units are routine in the art and include viral particle number determination, fluorescence microscopy, and titer by plaque assay. For example, the number of adenovirus particles can be determined by measuring the absorbance at A260. Similarly, infectious units can also be determined by quantitative immunofluorescence of vector specific proteins using monoclonal antibodies or by plaque assay.

In some embodiments, methods that calculate the infectious units include the plaque assay, in which titrations of the virus are grown on cell monolayers and the number of plaques is counted after several days to several weeks. For example, the infectious titer is determined, such as by plaque assay, for example an assay to assess cytopathic effects (CPE). In some embodiments, a CPE assay is performed by serially diluting virus on monolayers of cells, such as HFF cells, that are overlaid with agarose. After incubation for a time period to achieve a cytopathic effect, such as for about 3 to 28 days, generally 7 to 10 days, the cells can be fixed and foci of absent cells visualized as plaques are determined. In some embodiments, infectious units can be determined using an endpoint dilution (TCID₅₀) method, which determines the dilution of virus at which 50% of the cell cultures are infected and hence, generally, can determine the titer within a certain range, such as one log.

In some embodiments, the dosage of administration of a viral vector or virus-like particle is from about 10⁴ to about 10¹⁰ plaque forming units (pfu), inclusive. In some embodiments, the dosage of administration of a viral vector or virus-like particle is from about 10⁹ to about 10¹⁵ pfu, inclusive In some embodiments, the dosage of administration of a viral vector or virus-like particle is from about 10⁵ to about 10⁹ pfu. In some embodiments, the dosage of administration of a viral vector or virus-like particle is from about 10⁶ to about 10⁹ pfu. In some embodiments, the dosage of administration of a viral vector or virus-like particle is from about 10¹² to about 10¹⁴ pfu, inclusive. In some embodiments, the dosage of administration is 1.0×10⁹ pfu, 5.0×10⁹ pfu, 1.0×10¹⁰ pfu, 5.0×10¹⁰ pfu, 1.0×10¹¹ pfu, 5.0×10¹¹ pfu, 1.0×10¹² pfu, 5.0×10¹² pfu, or 1.0×10¹³ pfu, 5.0×10¹³ pfu, 1.0×10¹⁴ pfu, 5.0×10¹⁴ pfu, or 1.0×10¹⁵ pfu.

In some embodiments, the subject will receive a single injection. In some embodiments, administration can be repeated at daily/weekly/monthly intervals for an indefinite period and/or until the efficacy of the treatment has been established. As set forth herein, the efficacy of treatment can be determined by evaluating the symptoms and clinical parameters described herein and/or by detecting a desired response.

The exact amount of vehicle provided lipid particle required will vary from subject to subject, depending on the species, age, weight and general condition of the subject, the particular polynucleic acid, polypeptide, or vector used, its mode of administration etc. TAn appropriate amount can be determined by one of ordinary skill in the art using only routine experimentation given the teachings herein.

Compositions in some embodiments are provided as sterile liquid preparations, e.g., isotonic aqueous solutions, suspensions, emulsions, dispersions, or viscous compositions, which may in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the lipid particles in a solvent, such as in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts may in some aspects be consulted to prepare suitable preparations.

Injectables can be prepared in conventional forms, either as liquid solutions or suspensions, solid forms suitable for solution of suspension in liquid prior to injection, or as emulsions. As used herein, “parenteral administration” includes intradermal, intranasal, subcutaneous, intramuscular, intraperitoneal, intravenous and intratracheal routes, as well as a slow release or sustained release system such that a constant dosage is maintained.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin.

Sustained-release preparations may be prepared. Suitable examples of sustained-release preparations include semipermeable matrices of solid hydrophobic polymers containing the antibody, which matrices are in the form of shaped articles, e.g. films, or microcapsules.

In some embodiments, vehicle formulations may comprise cryoprotectants. As used herein, there term “cryoprotectant” refers to one or more agent that when combined with a given substance, helps to reduce or eliminate damage to that substance that occurs upon freezing. In some embodiments, cryoprotectants are combined with vector vehicles in order to stabilize them during freezing. In some aspects, Frozen storage of RNA between −20° C. and −80° C. may be advantageous for long term (e.g. 36 months) stability of polynucleotide. In some embodiments, the RNA species is mRNA. In some embodiments, cryoprotectants are included in vehicle formulations to stabilize polynucleotide through freeze/thaw cycles and under frozen storage conditions. Cryoprotectants of the provided embodiments may include, but are not limited to sucrose, trehalose, lactose, glycerol, dextrose, raffinose and/or mannitol. Trehalose is listed by the Food and Drug Administration as being generally regarded as safe (GRAS) and is commonly used in commercial pharmaceutical formulations.

The formulations to be used for in vivo administration are generally sterile. Sterility may be readily accomplished, e.g., by filtration through sterile filtration membranes.

### V. Methods of Use and Therapeutic Applications

In some embodiments, the lipid particles (e.g. lentiviral particles) containing a truncated BaEV envelope glycoprotein provided herein, are used for delivery of an exogenous agent to a target cell. The exogenous agent can be a protein, nucleic acid, such as DNA or RNA (e.g., mRNA), or small molecule. Exemplary exogenous agents that can be contained in a non-cell particle herein for delivery are described. Among provided methods herein are methods that comprise delivering an exogenous agent to a target cell. In some embodiments, the exogenous agent is an agent that is entirely heterologous or not produced or normally expressed by the target cell.

In some embodiments, delivery is by transduction of a provided lentiviral vector particle into the target cell. Hence, also provided herein are methods of transduction of a target cells with a provided lentiviral vector particle pseudotyped with a truncated BaEV envelope glycoprotein. comprising contacting the hematopoietic cell with a pseudotyped viral vector particle as defined above under conditions to effect the transduction of the hematopoietic cell by the pseudotyped viral vector particle. In some embodiments, transduction with a viral vector particle (e.g. lentiviral vector particle) initially delivers the biological material to the membrane or the cytoplasm of the target cell, upon being bound to the target cell. After delivery, the biological material can be translocated to other compartment of the cell. In some embodiments, transduction mediates integration of an exogenous gene expressed by the particle into the genome of the cell. Conditions to effect the transduction of the targeted cells are well-known from the skilled person and include typically incubating the cells to be transduced, such as by culture in flasks, plates or dishes an in some cases in the presence of a transduction adjuvant (e.g. retronectin). In some embodiments, the target cells may be prestimulated or activated, such as with cytokine cocktails or other stimulatory agents for stimulating or activating the target cells. In some embodiments, the viral vector particles are incubated with the target cells at an MOI of 1, 5, 10 or 100, or any value between any of the foregoing. In some embodiments, the incubation is in serum-free medium.

In some embodiments, the target cell is a hematopoietic cell. In some embodiments, the hematopoietic cell is a blood cell, such as from the myeloid or the lymphoid lineage. In particular, the hematopoietic cell may be an undifferentiated or poorly differentiated cells such as hematopoietic stem cells and progenitor cells, or differentiated cells such as T lymphocytes, B lymphocytes or dendritic cells. In some embodiments, the hematopoietic cell is selected from the group consisting of hematopoietic stem cells, CD34+ progenitor cells, in particular peripheral blood CD34+ cells, very early progenitor CD34+ cells, B-cell CD19+ progenitors, myeloid progenitor CD13+ cells, T lymphocytes, B lymphocytes, monocytes, dendritic cells, cancer B cells in particular B-cell chronic lymphocytic leukemia (BCLL) cells and marginal zone lymphoma (MZL) B cells, and thymocytes.

In some embodiments, the target cell is a hematopoietic stem cell (HSC). HSCs are stem cells that replenish all blood cell types and to self-renew. Hematopoietic stem cells may be in particular defined as cells that keep the levels of myeloid, T and B cells at robustly detectable levels (typically more than 1% of peripheral blood cells) for 16 weeks when injected into the circulation of a recipient mouse with a depleted hematopoietic system (Schroeder (2010) Cell Stem Cell 6:203-207).

In some embodiments, the target cell is a CD34+ progenitor cell. In some aspects, CD34+ progenitor cells are a heterogeneous cell population that includes a subpopulation of HSCs, pluripotent stem cells and cells in the early stages of lineage commitment. CD34+ progenitor cells continuously migrate to and from the bone marrow in normal adult animals. They can differentiate to produce all hematopoietic cell lineages found in the circulation.

In some embodiments, the target cell is a T cell. In some embodiments, the T cell is a resting or quiescent T cell. In some embodiments, the T cell is a naive or a memory T cell. In some embodiments, the T cell has not been activated prior to the delivery of the lipid particles, including prior to transduction with a provided lentiviral vector particle. Thus, in aspects of the provided methods, T cells are not activated with a T cell stimulatory agent such as with an anti-CD3/anti-CD28 antibody reagent (e.g. Dynabeads) prior to their transduction with a provided truncated BaEV envelope glycoprotein pseudotyped viral vector particle (e.g. lentiviral vector particle). The T cell may be a CD4+ T cell or a CD8+ T cell or a subset thereof.

In some embodiments, the target cell is a B cell. In some embodiments, the B cell is a resting B cell, such as a naïve ro memory B cell. In some embodiments, the B cell may be a cancer B cell, such as a B-cell chronic lymphocytic leukemia (BCLL) cell or a marginal zone lymphoma (MZL) B cell.

In some embodiments, delivery of the exogenous agent to the target cell can provide a therapeutic effect to treat a disease or condition in the subject. The therapeutic effect may be by targeting, modulating or altering an antigen or protein present or expressed by the target cell that is associated with or involved in a disease or condition. The therapeutic effect may be by providing an exogenous agent in which the exogenous agent is a protein (or a nucleic acid encoding the protein, e.g., an mRNA encoding the protein) which is absent, mutant, or at a lower level than wild-type in the target cell. In some embodiments, the target cell is from a subject having a genetic disease, e.g., a monogenic disease, e.g., a monogenic intracellular protein disease.

In some embodiments, the target cell is from a subject having a hematopoietic disease or disorder. In some embodiments, the hematopoietic disorder may be due to a blood disease, in particular disease involving hematopoietic cells. In some embodiments, the hematopoietic disorder is a monogenic hematopoietic disease, such as due to mutation of a single gene. In some embodiments, the hematopoietic disorder is myelodysplasia, aplastic anemia, Fanconi anemia, paroxysmal nocturnal hemoglobinuria, Sickle cell disease, Diamond Blackfan anemia, Schachman Diamond disorder, Kostmann's syndrome, chronic granulomatous disease, adrenoleukodystrophy, leukocyte adhesion deficiency, hemophilia, thalassemia, beta-thalassemia, leukaemia such as acute lymphocytic leukemia (ALL), acute myelogenous (myeloid) leukemia (AML), adult lymphoblastic leukaemia, chronic lymphocytic leukemia (CLL), B-cell chronic lymphocytic leukemia (B-CLL), chronic myeloid leukemia (CML), juvenile chronic myelogenous leukemia (CML), and juvenile myelomonocytic leukemia (JMML), severe combined immunodeficiency disease (SCID), X-linked severe combined immunodeficiency, Wiskott-Aldrich syndrome (WAS), adenosine-deaminase (ADA) deficiency, chronic granulomatous disease, Chediak-Higashi syndrome, Hodgkin's lymphoma, non-Hodgkin's lymphoma (NHL) or AIDS.

In some embodiments, the target cell is from a subject having an autoimmune disease. In some embodiments, the autoimmune disease is acute disseminated encephalomyelitis, acute hemorrhagic leukoencephalitis, Addison's disease, Agammaglobulinemia, Alopecia areata, amyotrophic lateral sclerosis, ankylosing spondylitis, antiphospholipid syndrome, antisynthetase syndrome, atopic allergy, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenia purpura, autoimmune urticaria, autoimmune uveitis, Balo disease, Balo concentric sclerosis, Bechets syndrome, Berger's disease, Bickerstaff's encephalitis, Blau syndrome, bullous pemphigoid, cancer, Castleman's disease, celiac disease, chronic inflammatory demyelinating polyneuropathy, chronic recurrent multifocal osteomyelitis, Churg-Strauss syndrome, cicatricial pemphigoid, Cogan syndrome, cold agglutinin disease, complement component 2 deficiency, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, discoid lupus erythematosus, eczema, enthesitis-related arthritis, eosinophilic fasciitis, eosinophilic gastroenteritis, epidermolysis bullosa acquisita, erythema nodosum, essential mixed cryoglobulinemia, Evan's syndrome, firodysplasia ossificans progressiva, fibrosing aveolitis, gastritis, gastrointestinal pemphigoid, giant cell arteritis, glomerulonephritis, goodpasture's syndrome, Grave's disease, Guillain-Barre syndrome (GBS), Hashimoto's encephalitis, Hashimoto's thyroiditis, hemolytic anaemia, Henoch-Schonlein purpura, herpes gestationis, hypogammaglobulinemia, idiopathic inflammatory demyelinating disease, idiopathic pulmonary fibrosis, idiopathic thrombocytopenia purpura, IgA nephropathy, inclusion body myositis, inflammatory demyelinating polyneuropathy, interstitial cystitis, juvenile idiopathic arthritis, juvenile rheumatoid arthritis, Kawasaki's disease, Lambert-Eaton myasthenic syndrome, leukocytoclastic vasculitis, lichen planus, lichen sclerosus, linear IgA disease (LAD), Lou Gehrig's disease, lupoid hepatitis, lupus erythematosus, Majeed syndrome, Meniere's disease, microscopic polyangiitis, Miller-Fisher syndrome, mixed connective tissue disease, morphea, Mucha-Habermann disease, multiple sclerosis, myasthenia gravis, myositis, neuropyelitis optica, neuromyotonia, ocular cicatricial pemphigoid, opsoclonus myoclonus syndrome, ord thyroiditis, palindromic rheumatism, paraneoplastic cerebellar degeneration, paroxysmal nocturnal hemoglobinuria (PNH), Parry Romberg syndrome, Parsonnage-Turner syndrome, pars planitis, pemphigus, pemphigus vulgaris, permicious anemia, perivenous encephalomyelitis, POEMS syndrome, polyarteritis nodosa, polymyalgia rheumatica, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatoid fever, sarcoidosis, Schmidt syndrome, Schnitzler syndrome, scleritis, scleroderma, Sjogren's syndrome, spondylarthropathy, Still's disease, stiff person syndrome, subacute bacterial endocarditis, Susac's syndrome, Sweet's syndrome, Sydenham chorea, sympathetic ophthalmia, Takayasu's arteritis, temporal arteritis, Tolosa-Hunt syndrome, transverse myelitis, ulcerative colitis, undifferentiated connective tissue disease, undifferentiated spondylarthropathy, vasculitis, vitiligo or Wegener's granulomatosis.

In some embodiments, the target cell is from a subject having a cancer. In some embodiments, the cancer is leukemia. In some embodiments, the leukemia is B-CLL, CML or T cell based leukemia such as ALT. In some embodiments, the cancer is a melanoma.

In some embodiments, the target cell is from a subject having a demyelinating disease of the central nervous system.

The lipid particles, e.g., lentiviral vectors, or pharmaceutical compositions containing the same, described herein can be administered to a subject, e.g., a mammal, e.g., a human. In such embodiments, the subject may be at risk of, may have a symptom of, or may be diagnosed with or identified as having, a particular disease or condition (e.g., a disease or condition described herein). In some embodiments, the disease or condition may be one that is treated by delivery of the exogenous agent contained in the administered lipid particle to a target cell in the subject.

In some embodiments, this disclosure provides, in certain aspects, a method of administering a lipid particle composition to a subject (e.g., a human subject), comprising administering to the subject, a provided lipid particle composition comprising a plurality of lipid particles described herein, thereby administering the lipid particle composition to the subject.

In some embodiments, this disclosure provides, in certain aspects, a method of delivering a lipid particle composition to target cells, comprising contacting a target cell with a provided lipid particle composition comprising a plurality of lipid particles described herein, thereby delivering the lipid particle composition to the target cell. In some embodiments, the contacting is carried out by administering a provided lipid particle to a subject, in which the lipid particle is delivered to the target cell present in the subject.

In some embodiments, the disclosure provides, in certain aspects, a method of delivering an exogenous agent, for instance a therapeutic agent (e.g., a polypeptide, a nucleic acid, a metabolite, an organelle, or a subcellular structure), to a subject or a cell, comprising administering to the subject, a plurality of lipid particles described herein, or a pharmaceutical composition described herein, wherein the lipid particle composition is administered in an amount and/or time such that the therapeutic agent is delivered. Exemplary exogenous agents that can be contained in a lipid particle herein for delivery to a subject are described in Section III.D

In some embodiments, the disclosure provides, in certain aspects, a method of delivering an exogenous agent, for instance a therapeutic agent (e.g., a polypeptide, a nucleic acid, a metabolite, an organelle, or a subcellular structure), to a target cell, comprising contacting a target cells with a plurality of lipid particles described herein, or a pharmaceutical composition described herein, wherein the lipid particle composition is contacted with the target cell under conditions such that the therapeutic agent is delivered. Exemplary exogenous agents that can be contained in a lipid particle herein for delivery to a subject are described in Section III.D. In some embodiments, the contacting is carried out by administering a provided lipid particle to a subject, in which the therapeutic agent (e.g. exogenous agent) contained in the lipid particle is delivered to the target cell present in the subject.

In some embodiments, delivery of an exogenous agent by administration of a lipid particle composition described herein may modify cellular protein expression levels. In certain embodiments, the administered composition directs upregulation of (via expression in the cell, delivery in the cell, or induction within the cell) of one or more exogenous agent cargo (e.g., a polypeptide or mRNA) that provide a functional activity which is substantially absent or reduced in the cell in which the polypeptide is delivered. In some embodiments, the missing functional activity may be enzymatic, structural, or regulatory in nature. In some embodiments, the administered composition directs up-regulation of one or more polypeptides that increases (e.g., synergistically) a functional activity which is present but substantially deficient in the cell in which the polypeptide is upregulated. In some of any embodiments, the administered composition directs downregulation of (via expression in the cell, delivery in the cell, or induction within the cell) of one or more cargo (e.g., a polypeptide, siRNA, or miRNA) that repress a functional activity which is present or upregulated in the cell in which the polypeptide, siRNA, or miRNA is delivered. In some of any embodiments, the upregulated functional activity may be enzymatic, structural, or regulatory in nature. In some embodiments, the administered composition directs down-regulation of one or more polypeptides that decreases (e.g., synergistically) a functional activity which is present or upregulated in the cell in which the polypeptide is downregulated. In some embodiments, the administered composition directs upregulation of certain functional activities and downregulation of other functional activities.

In some of any embodiments, the lipid particle composition (e.g., one comprising mitochondria or DNA) mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the lipid particle composition comprises an exogenous protein), the effect lasts for less than 1, 2, 3, 4, 5, 6, or 7 days, 2, 3, or 4 weeks, or 1, 2, 3, 6, or 12 months.

In some embodiments, the lipid particle further comprises, or the method further comprises delivering, a second exogenous agent that comprises or encodes a second cell surface ligand or antibody that binds a cell surface receptor, and optionally further comprising or encoding one or more additional cell surface ligands or antibodies that bind a cell surface receptor (e.g., 1, 2, 3, 4, 5, 10, 20, 50, or more). In some embodiments, the first exogenous agent and the second exogenous agent form a complex, wherein optionally the complex further comprises one or more additional cell surface ligands. In some embodiments, the exogenous agent comprises or encodes a cell surface receptor, e.g., an exogenous cell surface receptor. In some embodiments, the lipid particle further comprises, or the method further comprises delivering, a second exogenous agent that comprises or encodes a second cell surface receptor, and optionally further comprises or encodes one or more additional cell surface receptors (e.g., 1, 2, 3, 4, 5, 10, 20, 50, or more cell surface receptors).

In some embodiments, the lipid particle is capable of delivering (e.g., delivers) one or more cell surface receptors to a target cell (e.g., an immune cell). Similarly, in some embodiments, a method herein comprises delivering one or more cell surface receptors to a target cell. In some embodiments, the first exogenous agent and the second exogenous agent form a complex, wherein optionally the complex further comprises one or more additional cell surface receptors. In some embodiments, the exogenous agent comprises or encodes an antigen or an antigen presenting protein.

In some embodiments, the lipid particle is capable of causing (e.g., causes) a target cell to secrete a protein, e.g., a therapeutic protein. In some embodiments, the lipid particle is capable of delivering (e.g., delivers) a secreted exogenous agent, e.g., a secreted protein to a target site (e.g., an extracellular region), e.g., by delivering a nucleic acid (e.g., mRNA) encoding the protein to the target cell under conditions that allow the target cell to produce and secrete the protein. Similarly, in some embodiments, a method herein comprises delivering a secreted exogenous agent as described herein. In embodiments, the secreted protein comprises a protein therapeutic, e.g., an antibody molecule, a cytokine, or an enzyme. In embodiments, the secreted protein comprises an autocrine signaling molecule or a paracrine signaling molecule. In embodiments, the secreted exogenous agent comprises a secretory granule.

In some embodiments, the lipid particle is capable of secreting (e.g., secretes) an exogenous agent, e.g., a protein. In some embodiments, the exogenous agent, e.g., secreted agent, is delivered to a target site in a subject. In some embodiments, the exogenous agent is a protein that cannot be made recombinantly or is difficult to make recombinantly. In some embodiments, the lipid particle that secretes a protein is from a source cell selected from an MSC or a chondrocyte.

In some embodiments, the lipid particle is capable of reprogramming (e.g., reprograms) a target cell (e.g., an immune cell), e.g., by delivering an exogenous agent selected from a transcription factor, a nucleic acid encoding a transcription factor, mRNA, or a plurality of said exogenous agents. Similarly, in some embodiments, a method herein comprises reprogramming a target cell. In embodiments, reprogramming comprises inducing an exhausted T cell to take on one or more characteristics of a nonexhausted T cell, e.g., a killer T cell. In some embodiments, the exogenous agent comprises an antigen. In some embodiments, the lipid particle comprises a first exogenous agent comprising an antigen and a second exogenous agent comprising an antigen presenting protein.

In some embodiments, a lipid particle is capable of modifying, e.g., modifies, a target tumor cell, for instance by delivering an exogenous agent (protein or nucleic acid) or a nucleic encoding an exogenous agent. Similarly, in some embodiments, a method herein comprises modifying a target tumor cell. In embodiments, the lipid particle delivers an mRNA encoding an immunostimulatory ligand, an antigen presenting protein, a tumor suppressor protein, or a pro-apoptotic protein. In some embodiments, the lipid particle delivers an miRNA capable of reducing levels in a target cell of an immunosuppressive ligand, a mitogenic signal, or a growth factor.

In some embodiments, a lipid particle delivers an exogenous agent that is immunomodulatory, e.g., immunostimulatory.

In some embodiments, a lipid particle is capable of causing (e.g., causes) the target cell to present an antigen, for instance by delivering an exogenous agent comprising an antigen or a nucleic acid encoding the antigen. Similarly, in some embodiments, a method herein comprises presenting an antigen on a target cell. In some embodiments, the lipid particle promotes regeneration in a target tissue. Similarly, in some embodiments, a method herein comprises promoting regeneration in a target tissue.

In some embodiments, the lipid particle is capable of delivering (e.g., delivers) a nucleic acid to a target cell, e.g., to stably modify the genome of the target cell, e.g., for gene therapy. Similarly, in some embodiments, a method herein comprises delivering a nucleic acid to a target cell. In some embodiments, the target cell has an enzyme deficiency, e.g., comprises a mutation in an enzyme leading to reduced activity (e.g., no activity) of the enzyme.

In some embodiments, the lipid particle is capable of delivering (e.g., delivers) a reagent that mediates a sequence specific modification to DNA (e.g., Cas9, ZFN, or TALEN) in the target cell. Similarly, in some embodiments, a method herein comprises delivering the reagent to the target cell. In embodiments, the target cell is a CNS cell.

In some embodiments, the lipid particle is capable of delivering (e.g., delivers) a nucleic acid to a target cell, e.g., to transiently modify gene expression in the target cell.

In some embodiments, the lipid particle is capable of delivering (e.g., delivers) a protein to a target cell, e.g., to transiently rescue a protein deficiency. Similarly, in some embodiments, a method herein comprises delivering a protein to a target cell. In embodiments, the protein is a membrane protein (e.g., a membrane transporter protein), a cytoplasmic protein (e.g., an enzyme), or a secreted protein (e.g., an immunosuppressive protein).

In some embodiments, the lipid particle is capable of intracellular molecular delivery, e.g., delivers a protein exogenous agent to a target cell. Similarly, in some embodiments, a method herein comprises delivering a molecule to an intracellular region of a target cell. In embodiments, the protein exogenous agent is an inhibitor. In some embodiments, the protein exogenous agent comprises a nanobody, scFv, camelid antibody, peptide, macrocycle, or small molecule.

In some embodiments, the lipid particle comprises on its membrane one or more cell surface ligands (e.g., 1, 2, 3, 4, 5, 10, 20, 50, or more cell surface ligands), said cell surface ligands to be presented by the lipid particle to a target cell. Similarly, in some embodiments, a method herein comprises presenting one or more cell surface ligands to a target cell. In some embodiments, the lipid particle having a cell surface ligand is from a source cell chosen from a neutrophil (e.g., and the target cell is a tumor-infiltrating lymphocyte), dendritic cell (e.g., and the target cell is a naive T cell), or neutrophil (e.g., and the target is a tumor cell or virus-infected cell). In some embodiments the lipid particle comprises a membrane complex, e.g., a complex comprising at least 2, 3, 4, or 5 proteins, e.g., a homodimer, heterodimer, homotrimer, heterotrimer, homotetramer, or heterotetramer. In some embodiments, the lipid particle comprises an antibody, e.g., a toxic antibody, e.g., the lipid particle is capable of delivering the antibody to the target site, e.g., by homing to a target site. In some embodiments, the source cell is an NK cell or neutrophil.

In some embodiments, a method herein comprises causing ligand presentation on the surface of a target cell by presenting cell surface ligands on the lipid particle. In some embodiments, the lipid particle is capable of causing cell death of the target cell. In some embodiments, the lipid particle is from a NK source cell. In some embodiments, a lipid particle or target cell is capable of phagocytosis (e.g., of a pathogen). Similarly, in some embodiments, a method herein comprises causing phagocytosis. In some embodiments, a lipid particle senses and responds to its local environment. In some embodiments, the lipid particle is capable of sensing level of a metabolite, interleukin, or antigen.

In embodiments, a lipid particle is capable of chemotaxis, extravasation, or one or more metabolic activities. In embodiments, the metabolic activity is selected from kyneurinine, gluconeogenesis, prostaglandin fatty acid oxidation, adenosine metabolism, urea cycle, and thermogenic respiration. In some embodiments, the source cell is a neutrophil and the lipid particle is capable of homing to a site of injury. In some embodiments, the source cell is a macrophage and the lipid particle is capable of phagocytosis. In some embodiments, the source cell is a brown adipose tissue cell and the lipid particle is capable of lipolysis.

In some embodiments, the lipid particle comprises (e.g., is capable of delivering to the target cell) a plurality of exogenous agents (e.g., at least 2, 3, 4, 5, 10, 20, or 50 exogenous agents) or nucleic acids encoding a plurality of exogenous agents. In embodiments, the lipid particle comprises an inhibitory nucleic acid (e.g., siRNA or miRNA) and an mRNA.

In some embodiments, the lipid particle comprises (e.g., is capable of delivering to the target cell) a membrane protein or a nucleic acid encoding the membrane protein. In embodiments, the lipid particle is capable of reprogramming or transdifferentiating a target cell, e.g., the lipid particle comprises one or more agents that induce reprogramming or transdifferentiation of a target cell.

### VI. EXEMPLARY EMBODIMENTS

Among provided embodiments are:

1. A Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEv envelope glycoprotein.

2. A Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein.

3. The lentiviral particle of embodiment 1 or embodiment 2 that is replication defective.

4. The lentiviral particle of any of embodiments 1-3 prepared by a method comprising transducing a producer cell with packaging plasmids that encode a Gag-pol, Rev, Tat and the truncated BaEV envelope glycoprotein.

5. The lentiviral particle of any of embodiments 1-4, wherein the lentiviral particle further comprises a viral nucleic acid.

6. The lentiviral particle of embodiment 5, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

7 The lentiviral particle of any of embodiments 1-4, wherein the lentiviral particle is devoid of viral genomic DNA.

8. A lipid particle comprising a truncated Baboon Endogenous Retrovirus (BaEV) envelope glycoprotein, comprising:
(a) a lipid bilayer enclosing a lumen, and
(b) a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one contiguous amino-terminal amino acids but less than the full length of the inhibitory R peptide of the inhibitory R peptide of the wild-type BaEV envelope glycoprotein, wherein the envelope glycoprotein is embedded in the lipid bilayer.

9. A lipid particle comprising a truncated Baboon Endogenous Retrovirus (BaEV) envelope glycoprotein, comprising:
(a) a lipid bilayer enclosing a lumen, and
(b) a truncated BaEV envelope glycoprotein comprising a cytoplasmic tail with a partial fusion inhibitory R peptide relative to the inhibitory R peptide of a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein, wherein the envelope glycoprotein is embedded in the lipid bilayer.

10. The lipid particle of embodiment 8 or embodiment 9, wherein the lipid bilayer is derived from a membrane of a host cell used for producing a retrovirus or retrovirus-like particle.

11. The lipid particle of embodiment 10, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

12. The lipid particle of any of embodiments 8-11, wherein the lipid bilayer is or comprises one or more other viral components other than the BaEV envelope glycoprotein.

13. The lipid particle of embodiment 12, wherein the one or more viral components are from a retrovirus.

14. The lipid particle of 10-13, wherein the retrovirus is a lentivirus or a lentivirus like particle.

15. The lentiviral particle of any of embodiments 1-7 or the lipid particle of an of embodiments 8-14, wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

16. The lentiviral particle or lipid particle of embodiment 15, wherein the glycoprotein 70 (g70) subunit or a biologically active portion thereof, and the portion of the glycoprotein p20E (p20E) subunit are associated via an inter-subunit disulfide bond.

17. The lentiviral particle of lipid particle of any of embodiments 1-16, wherein the BaEV glycoprotein binds an ASCT-2 or ASCT-1 receptor.

18. The lentiviral particle or lipid particle of any of embodiments 15-17, wherein the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25.

19. The lentiviral particle or lipid particle of any of embodiments 15-18, wherein the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

20. The lentiviral particle or lipid particle of any of embodiments 1-19, wherein the truncated BaEV envelope glycoprotein is truncated relative to SEQ ID NO:24 and lacks up to 16 contiguous amino acids from the C-terminal cytoplasmic tail of SEQ ID NO:24.

21. The lentiviral particle or lipid particle of any of embodiments 1-20, wherein the truncated BaEV envelope glycoprotein is truncated relative to SEQ ID NO:24 and lacks from 8 to 14, contiguous amino acids from the C-terminal cytoplasmic tail of SEQ ID NO:24, optionally from 8 to 13 contiguous amino acids from the C-terminal cytoplasmic tail of SEQ ID NO:24.

22. The lentiviral particle or lipid particle of any of embodiments 1, 3-7, 8 and 10-19, wherein the partial fusion inhibitory R peptide is set forth as amino acids 1 to 9 of SEQ ID NO:22, optionally wherein the cytoplasmic tail is set forth in SEQ ID NO:14 (R+9).

23. The lentiviral particle or lipid particle of any of embodiments 1, 3-7, 8 and 10-22, wherein the truncated BaEV envelope glycoprotein is set forth in SEQ ID NO: 37.

24. The lentiviral particle or lipid particle of any of embodiments 1-19, wherein the partial fusion inhibitory R peptide is set forth as amino acids 1 to 8 of SEQ ID NO:22, optionally wherein the cytoplasmic tail is set forth in SEQ ID NO:13 (R+8).

25. The lentiviral particle or lipid particle of any of embodiments 1-19 and 24, wherein the truncated BaEV envelope glycoprotein is set forth in SEQ ID NO: 36.

26. The lentiviral particle or lipid particle of any of embodiments 1, 3-7, 8 and 10-19, wherein the partial fusion inhibitory R peptide is set forth as amino acids 1 to 7 of SEQ ID NO:22, optionally wherein the cytoplasmic tail is set forth in SEQ ID NO: 12 (R+7).

27. The lentiviral particle or lipid particle of any of embodiments 1, 3-7, 8, 10-19 and 26, wherein the truncated BaEV envelope glycoprotein is set forth in SEQ ID NO: 35.

28. The lentiviral particle or lipid particle of any of embodiments 1, 3-7, 8, and 10-19, wherein the partial fusion inhibitory R peptide is set forth as amino acids 1 to 6 of SEQ ID NO:22, optionally wherein the cytoplasmic tail is set forth in SEQ ID NO:11 (R+6).

29. The lentiviral particle or lipid particle of any of embodiments 1, 3-7, 8, 10-19 and 28, wherein the truncated BaEV envelope glycoprotein is set forth in SEQ ID NO: 34.

30. The lentiviral particle or lipid particle of any of embodiments 1, 3-7, 8, and 10-19, wherein the partial fusion inhibitory R peptide is set forth as amino acids 1 to 5 of SEQ ID NO:22, optionally wherein the cytoplasmic tail is set forth in SEQ ID NO:10 (R+5).

31. The lentiviral particle or lipid particle of any of embodiments 1, 3-7, 8, 10-19 and 30, wherein the truncated BaEV envelope glycoprotein is set forth in SEQ ID NO: 33.

32. The lentiviral particle or lipid particle of any of embodiments 1, 3-7, 8, and 10-19, wherein the partial fusion inhibitory R peptide is set forth as amino acids 1 to 4 of SEQ ID NO:22, optionally wherein the cytoplasmic tail is set forth in SEQ ID NO:9 (R+4).

33. The lentiviral particle or lipid particle of any of embodiments 1, 3-7, 8, 10-19 and 32, wherein the truncated BaEV envelope glycoprotein is set forth in SEQ ID NO: 32.

34. The lentiviral particle or lipid particle of any of embodiments 1, 3-7, 8, and 10-19, wherein the partial fusion inhibitory R peptide is set forth as amino acids 1 to 3 of SEQ ID NO:22, optionally wherein the cytoplasmic tail is set forth in SEQ ID NO:8 (R+3).

35. The lentiviral particle or lipid particle of any of embodiments 1, 3-7, 8, 10-19 and 34, wherein the truncated BaEV envelope glycoprotein is set forth in SEQ ID NO: 31.

36. The lentiviral particle or lipid particle of any of embodiments 1-35, wherein the particle further comprises an exogenous agent.

37. The lentiviral particle or lipid particle of embodiment 36, wherein the exogenous agent is present in the lumen.

38. The lentiviral particle or lipid particle of embodiment 36 or embodiment 37, wherein the exogenous agent is a protein or a nucleic acid, optionally wherein the nucleic acid is a DNA or RNA.

39. The lentiviral particle of any of embodiments 36-38, wherein the exogenous agent is or encodes a factor associated with gene editing.

40. The lentiviral particle of any of embodiments 36-39, wherein the exogenous agent is or encodes a factor associated with base editing and/or prime editing (i.e, target-primed reverse transcription (TPRT)).

41. The lentiviral particle of any of embodiments 36-40, wherein the exogenous agent is or encodes a nuclease, optionally wherein the nuclease is selected from the group comprising a zinc-finger nucleases (ZFNs), transcription-activator like effector nucleases (TALENs), or a CRISPR-associated protein-nuclease (Cas)

42. The lentiviral particle of any of embodiments 36-40, wherein the exogenous agent is or encodes a transposase and/or recombinase.

43. The lentiviral particle of any of embodiments 36-40, wherein the exogenous agent is or encodes a DNA polymerase, RNA polymerase, or reverse-transcriptase.

44. The lentiviral particle or lipid particle of any of embodiments 1-43, wherein the particle is produced as a preparation with increased titer compared to a reference particle preparation that is similarly produced but with incorporation of a BaEV envelope glycoprotein having a cytoplasmic tail with a full length R peptide or a portion of the R peptide of 10 or more amino acids in length.

45. The lentiviral particle or lipid particle of embodiment 44, wherein the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more.

46. The lentiviral particle or lipid of any of embodiments 1-45, wherein the titer in target cells, optionally HEK293 cells, following transduction is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL.

47. The lentiviral particle or lipid particle of any of embodiments 1-46, wherein the truncated BaEV envelope glycoprotein is present on the surface of the particle at a density of at least about (0.001, 0.002, 0.005, 0.01, 0.02, 0.05, 0.1, 0.2 or 0.5) truncated BaEV envelope glycoproteins/nm².

48. A truncated BaEV envelope glycoprotein comprising a cytoplasmic tail with a partial fusion inhibitory R peptide relative to a wild-type BaEV envelope glycoprotein, wherein the partial fusion inhibitory R peptide comprises at least one contiguous amino-terminal amino acid, but less than the full length, of the inhibitory R peptide of the wild-type BaEV envelope glycoprotein.

49 The truncated BaEV envelope glycoprotein of embodiment 48 wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

50. The truncated BaEV envelope glycoprotein of embodiment 49, wherein the glycoprotein 70 (g70) subunit or a biologically active portion thereof, and the portion of the glycoprotein p20E (p20E) subunit are associated via an inter-subunit disulfide bond.

51. The truncated BaEV envelope glycoprotein of any of embodiments 48-50, wherein the BaEV glycoprotein binds an ASCT-2 or ASCT-1 receptor.

52. The truncated BaEV glycoprotein of any of embodiments 48-51, wherein the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25.

53. The truncated BaEV glycoprotein of any of embodiments 48-52, wherein the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

54. The truncated BaEV envelope glycoprotein of any of embodiments 48-53, wherein the truncated BaEV envelope glycoprotein is truncated relative to SEQ ID NO:24 and lacks up to 16 contiguous amino acids from the C-terminal cytoplasmic tail of SEQ ID NO:24.

55. The truncated BaEV envelope glycoprotein of any of embodiments 48-54, wherein the truncated BaEV envelope glycoprotein is truncated relative to SEQ ID NO:24 and lacks from 8 to 14 contiguous amino acids from the C-terminal cytoplasmic tail of SEQ ID NO:24, optionally from 8 to 13 contiguous amino acids from the C-terminal cytoplasmic tail of SEQ ID NO:24.

56. The truncated BaEV envelope glycoprotein of any of embodiments 48-55, wherein the partial fusion inhibitory R peptide is set forth as amino acids 1 to 9 of SEQ ID NO:22, optionally wherein the cytoplasmic tail is set forth in SEQ ID NO:14 (R+9).

57. The truncated BaEV envelope glycoprotein of any of embodiments 48-56, wherein the truncated BaEV envelope glycoprotein is set forth in SEQ ID NO: 37.

58. The truncated BaEV envelope glycoprotein of any of embodiments 48-55, wherein the partial fusion inhibitory R peptide is set forth as amino acids 1 to 8 of SEQ ID NO:22, optionally wherein the cytoplasmic tail is set forth in SEQ ID NO:13 (R+8).

59. The truncated BaEV envelope glycoprotein of any of embodiments 48-55 and 58, wherein the truncated BaEV envelope glycoprotein is set forth in SEQ ID NO: 36.

60. The truncated BaEV envelope glycoprotein of any of embodiments 48-55, wherein the partial fusion inhibitory R peptide is set forth as amino acids 1 to 7 of SEQ ID NO:22, optionally wherein the cytoplasmic tail is set forth in SEQ ID NO: 12 (R+7).

61. The truncated BaEV envelope glycoprotein of any of embodiments 48-55 and 60, wherein the truncated BaEV envelope glycoprotein is set forth in SEQ ID NO: 35.

62. The truncated BaEV envelope glycoprotein of any of embodiments 48-55, wherein the partial fusion inhibitory R peptide is set forth as amino acids 1 to 6 of SEQ ID NO:22, optionally wherein the cytoplasmic tail is set forth in SEQ ID NO:11 (R+6).

63. The truncated BaEV envelope glycoprotein of any of embodiments 48-55 and 62, wherein the truncated BaEV envelope glycoprotein is set forth in SEQ ID NO: 34.

64. The truncated BaEV envelope glycoprotein of any of embodiments 48-55, wherein the partial fusion inhibitory R peptide is set forth as amino acids 1 to 5 of SEQ ID NO:22, optionally wherein the cytoplasmic tail is set forth in SEQ ID NO:10 (R+5).

65. The truncated BaEV envelope glycoprotein of any of embodiments 48-55 and 64, wherein the truncated BaEV envelope glycoprotein is set forth in SEQ ID NO: 33.

66. The truncated BaEV envelope glycoprotein of any of embodiments 48-55, wherein the partial fusion inhibitory R peptide is set forth as amino acids 1 to 4 of SEQ ID NO:22, optionally wherein the cytoplasmic tail is set forth in SEQ ID NO:9 (R+4).

67. The truncated BaEV envelope glycoprotein of any of embodiments 48-55 and 66, wherein the truncated BaEV envelope glycoprotein is set forth in SEQ ID NO: 32.

68. The truncated BaEV envelope glycoprotein of any of embodiments 48-55, wherein the partial fusion inhibitory R peptide is set forth as amino acids 1 to 3 of SEQ ID NO:22, optionally wherein the cytoplasmic tail is set forth in SEQ ID NO:8 (R+3).

69. The truncated BaEV envelope glycoprotein of any of embodiments 48-55 and 68, wherein the truncated BaEV envelope glycoprotein is set forth in SEQ ID NO: 31.

70. A polynucleotide comprising a nucleic acid encoding the truncated BaEV envelope glycoprotein of any of embodiments 48-69.

71. The polynucleotide of embodiment 70, wherein the polynucleotide is codon optimized.

72. The polynucleotide of embodiment 70 or embodiment 71, further comprising at least one promoter that is operatively linked to control expression of the nucleic acid.

73. The polynucleotide of embodiment 72, wherein the promoter is a constitutive promoter.

74. The polynucleotide of embodiment 72 or embodiment 73, wherein the promoter is an inducible promoter.

75. A vector, comprising the polynucleotide of any of embodiments 70-74.

76. A plasmid, comprising the polynucleotide of any of embodiments 70-74.

77. The plasmid of embodiment 76, further comprising one or more nucleic acids encoding proteins for lentivirus production.

78. A cell comprising the polynucleotide of any of embodiments 70-74, the vector of embodiment 75 or the plasmid of embodiment 76 or embodiment 77.

79. The cell of embodiment 78 that is a producer cell for production of a lentiviral particle.

80. A producer cell comprising (i) a viral nucleic acid(s) and (ii) a nucleic acid encoding the truncated BaEV envelope glycoprotein of any of embodiments 48-69, optionally wherein the viral nucleic acid(s) are lentiviral nucleic acids.

81. The producer cell of embodiment 80, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

82. The producer cell of embodiment 80 or embodiment 81, wherein the producer cell comprises 293T cells.

83. The producer cell of any of embodiments 80-82, wherein the viral nucleic acid(s) lacks one or more genes involved in viral replication.

84. The producer cell of any of embodiments 80-83, wherein the viral nucleic acid comprises a nucleic acid encoding a viral packaging protein selected from one or more of Gag, Pol, Rev and Tat.

85. The producer cell of any of embodiments 80-84, wherein the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

86. A method of making a lipid particle comprising a truncated BaEV glycoprotein comprising:
a) introducing into a source cell a polynucleotide of any of embodiments 70-74, the vector of embodiment 75, or the plasmid of embodiment 76 or embodiment 77;
b) culturing the cell under conditions that allow for production of a lipid particle, and c) separating, enriching, or purifying the lipid particle from the cell, thereby making the lipid particle.

87. The method of embodiment 86, wherein the source cell is a mammalian cell.

88. The method of embodiment 86 or embodiment 87, wherein the source cell is a producer cell and the lipid particle is a viral particle or a viral-like particle, optionally a retroviral particle or a retroviral-like particle, optionally a lentiviral particle or lentiviral-like particle.

89. A method of making a pseudotyped lentiviral particle, the method comprising:
a) providing a producer cell that comprises a lentiviral nucleic acid (s) and a nucleic acid encoding a truncated BaEV envelope glycoprotein of any of embodiments 48-69 or the polynucleotide of any of embodiments 70-74;
b) culturing the cell under conditions that allow for production of the lentiviral particle, and c) separating, enriching, or purifying the lentiviral particle from the producer cell, thereby making the pseudotyped lentiviral particle.

90. The method of embodiment 88 or embodiment 89, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

91. The method of any of embodiments 88-90, wherein the producer cell comprises 293T cells.

92. The method of any of embodiments 88-91, wherein the method produces a lentiviral preparation with increased titer compared to a reference lentiviral particle preparation that is similarly produced but is pseudotyped with a BaEV envelope glycoprotein having a cytoplasmic tail with a full length R peptide or a portion of the R peptide of 10 or more amino acids in length.

93. The method of embodiment 92, wherein the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more.

94. The method of any of embodiments 88-93, wherein the method produces a lentiviral preparation with a titer in target cells, optionally HEK293 cells, following transduction that is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL.

95. The method of any of embodiments 88-94, wherein the method results in reduced syncytia formation of the producer cell compared to a similar method but that is for production of a reference lentiviral particle preparation pseudotyped with a BaEV envelope glycoprotein having a cytoplasmic tail with no R peptide (Rless) or an R peptide of 3 contiguous amino terminal amino acids or less in length relative to the wild-type BaEV envelope glycoprotein R peptide.

96. The method of any of embodiments 88-95, wherein the method produces a lentiviral preparation with high titer (e.g. greater than 4×10⁶ TU/mL, greater than 5×10⁶ TU/mL, greater than 6×10⁶ TU/mL, greater than 7×10⁶ TU/mL, greater than 8×10⁶ TU/mL, greater than 9×10⁶ TU/mL, greater than 1×10⁷ TU/mL, or greater than 1.2×10⁷ TU/mL) and minimal syncytia formation of the producer cell during the method of production.

97. A lipid particle produced by the method of any of embodiments 86-88 and 90-96.

98. A lentiviral particle produced by the method of any of embodiments 89-96.

99. A lipid particle comprising the truncated BaEV envelope glycoprotein of any of embodiments 48-69.

100. A lentiviral particle pseudotyped with a truncated BaEV envelope glycoprotein of any of embodiments 48-69.

101. A composition comprising a plurality of the lentiviral particles of any of embodiments 1-7, 15-47, 98 and 100.

102. A composition comprising a plurality of the lipid particles of any of embodiments 8-47, 97 and 99.

103. The composition of embodiment 101 or embodiment 102 further comprising a pharmaceutically acceptable excipient.

104. A method of transducing a cell, the method comprising contacting a cell with the lentiviral particle of any of embodiments 1-7, 15-47, 98 and 100 or the composition of embodiment 101 or embodiment 103.

105. The method of embodiment 104, wherein the lipid particle or lentiviral vector comprises an exogenous agent and the transduction introduces the exogenous agent into the cell.

106. A method of delivering an exogenous agent into a cell, the method comprising contacting a lentiviral particle or lipid particle of any of embodiments 1-47 and 97-100 or the composition of any of embodiments 101-103 with a cell.

107. The method of any of embodiments 104-106, wherein the contacting is in vitro or ex vivo.

108. The method of any of embodiments 104-106, wherein the contacting is in vivo in a subject.

109. A method of delivering an exogenous agent to a cell in a subject, the method comprising administering to the subject the lentiviral particle or lipid particle of any of embodiments 1-47 and 97-100 or the composition of any of embodiments 101-103.

110. The method of any of embodiments 104-109, wherein the cell is a hematopoietic lineage cell.

111. The method of any of embodiments 104-110, wherein the cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells.

112. The method of any of embodiments 104-111, wherein the cell is selected from monocytes, macrophages, neutrophils, basophils, eosinophils, erythrocytes, megakaryocytes and platelets.

113. The method of any of embodiments 104-112, wherein the cell is selected from T cells, B cells, natural killer (NK) cells and innate lymphoid cells.

114. The method of any of embodiments 104-113, wherein the cell is a hematopoietic stem cell (HSC).

115. The method of embodiment 114, wherein the subject has received a hematopoietic stem cell transplant.

116. The method of any of embodiment 105-115, wherein the exogenous agent is a protein or a nucleic acid, optionally wherein the nucleic acid is a DNA or RNA.

117. The method of any of embodiments 105-116, wherein the exogenous agent is or encodes a therapeutic agent for treating a disease or condition in the subject.

118. The method of any of embodiments 105-116, wherein the exogenous agent is or encodes a membrane protein, optionally a chimeric antigen receptor, for targeting an antigen associated with a disease or condition in the subject.

119. The method of any of embodiments 105-117, wherein the exogenous agent is for use in gene therapy to correct a genetic deficiency or replaces a deficient or missing gene in the subject.

120. The method of any of embodiments 104-119, wherein the subject is a human subject.

### VII. Examples

The following examples are included for illustrative purposes only and are not intended to limit the scope of the invention.

### Example 1 Generation and Characterization of Lentiviruses Pseudotyped with Truncated BaEV Envelope Glycoproteins

This Example describes the generation and assessment of lentiviral vectors pseudotyped with exemplary truncated BaEV envelope glycoproteins.

### A. Generation of BaEV Pseudotyped Lentiviruses

Lentiviral vectors expressing enhanced green fluorescent protein (eGFP) were produced in 293T cells by transient transfection with the following plasmids: LTR backbone, eGFP under the control of a CMV promoter (CMV-eGFP); rev, tat, gag-pol, and a plasmid containing a nucleotide sequence encoding one of the truncated BaEV envelope glycoproteins summarized in Table E1. In this experiment, approximately 188 ng of the plasmid encoding the BaEV envelope glycoprotein was used per transfection. The encoded truncated BaEV envelope protein contained a truncated sequence relative to the full length BaEV envelope glycoprotein set forth in SEQ ID NO:24, in which the truncated BaEv envelope glycoprotein was generated to have a truncated cytoplasmic tail with a partial fusion inhibitory R peptide relative to the wildtype-cytoplasmic tail containing the full length R peptide of SEQ ID NO:24 (wild-type cytoplasmic tail containing full length R peptide set forth in SEQ ID NO:4).

TABLE El
Truncated BaEV envelope proteins

| | BaEv | | |
|---|---|---|---|
| | envelope | | |
| | glyco- | | |
| | protein | Cytoplasmic tail | |
| Partial fusion | SEQ | SEQ | |
| inhibitory R | ID | ID | |
| peptide | NO | NO | sequence |
| R-less | 28 | 5 | NRLTAFINDKLNIIHAM |
| R+1 | 29 | 6 | NRLTAFINDKLNIIHAMV |
| R+2 | 30 | 7 | NRLTAFINDKLNIIHAMVL |
| R+3 | 31 | 8 | NRLTAFINDKLNIIHAMVLT |
| R+4 | 32 | 9 | NRLTAFINDKLNIIHAMVLTQ |
| R+5 | 33 | 10 | NRLTAFINDKLNIIHAMVLTQQ |
| R+6 | 34 | 11 | NRLTAFINDKLNIIHAMVLTQQY |
| R+7 | 35 | 12 | NRLTAFINDKLNIIHAMVLTQQYQ |
| R+8 | 36 | 13 | NRLTAFINDKLNIIHAMVLTQQYQV |
| R+9 | 37 | 14 | NRLTAFINDKLNIIHAMVLTQQYQVL |
| R+10 | 38 | 15 | NRLTAFINDKLNIIHAMVLTQQYQVLR |
| R+11 | 39 | 16 | NRLTAFINDKLNIIHAMVLTQQYQVLRT |
| R+12 | 40 | 17 | NRLTAFINDKLNIIHAMVLTQQYQVLRTD |
| R+13 | 41 | 18 | NRLTAFINDKLNIIHAMVLTQQYQVLRTDE |
| R+14 | 42 | 19 | NRLTAFINDKLNIIHAMVLTQQYQVLRTDEE |
| R+15 | 43 | 20 | NRLTAFINDKLNIIHAMVLTQQYQVLRTDEEA |
| R+16 | 44 | 21 | NRLTAFINDKLNIIHAMVLTQQYQVLRTDEEAQ |
| Wild-type | 24 | 4 | NRLTAFINDKLNIIHAMVLTQQYQVLRTDEEAQD |
| cytoplasmic | | | |
| tail | | | |

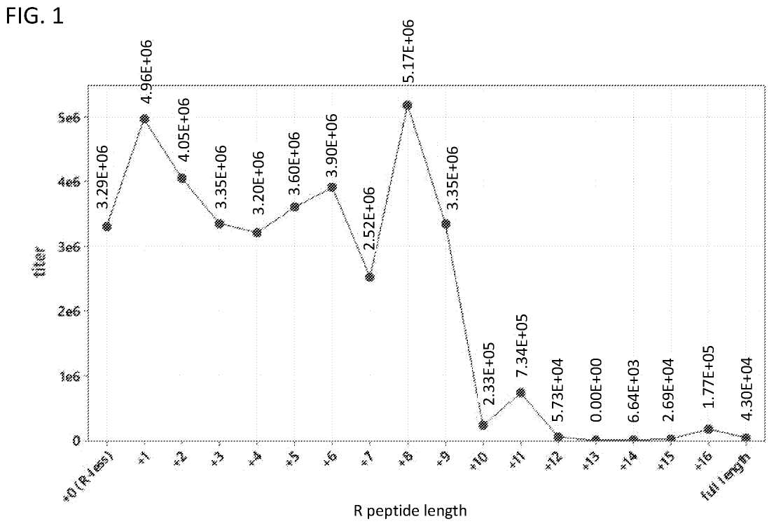

After 2 days, viral supernatants were harvested and frozen and the producer cells were imaged for EGFP. Subsequently, the supernatants were titered by thawing and applying them to a defined number of HEK293 cells in the presence of 5 ug/mL polybrene. After an additional 24-48 hours, these cells were trypsinized and EGFP was analyzed by flow cytometry. Titer was determined by % of cells that were GFP+. As shown in FIG. 1, cells transfected with constructs containing BaEV envelope glycoproteins containing between 1 and 9 contiguous amino-terminal amino acids of the inhibitory R peptide (R+1 to R+9) demonstrated increased titer over constructs containing between 10 and 16 contiguous amino-terminal amino acids, or the full length sequence, of the inhibitory R peptide.

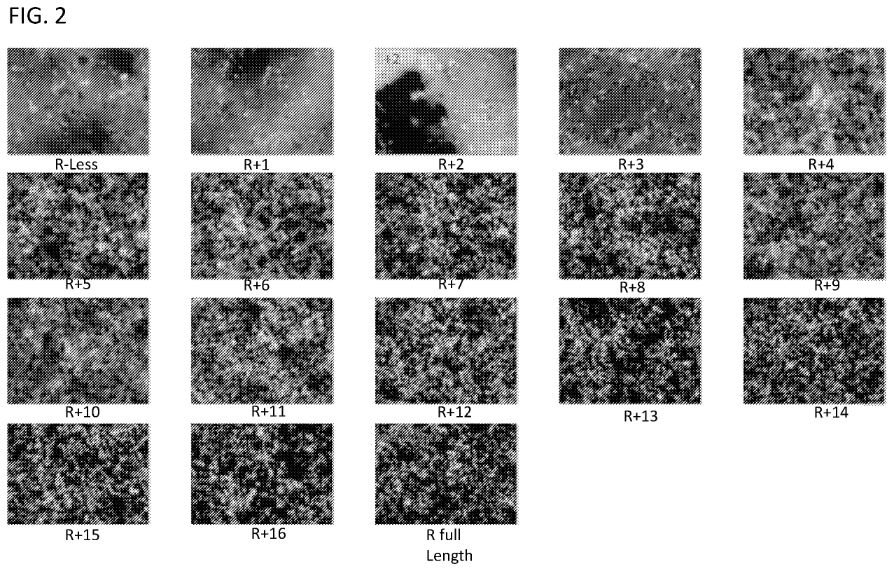

As shown in FIG. 2, syncytia formation was substantially reduced with constructs containing BaEV envelope glycoproteins containing between 4 and 9 contiguous amino-terminal amino acids of the inhibitory R peptide, whereas the R-less construct exhibited fusion of producer cells into large syncytia.

### Example 2 Optimization of Lentiviruses Pseudotyped with Truncated BaEV Envelope Glycoproteins

This Example describes the optimization of lentiviral vectors pseudotyped with the R+8 truncated BaEV envelope glycoprotein containing 8 contiguous amino acids of the R-peptide (SEQ ID NO:13) described in Example 1.

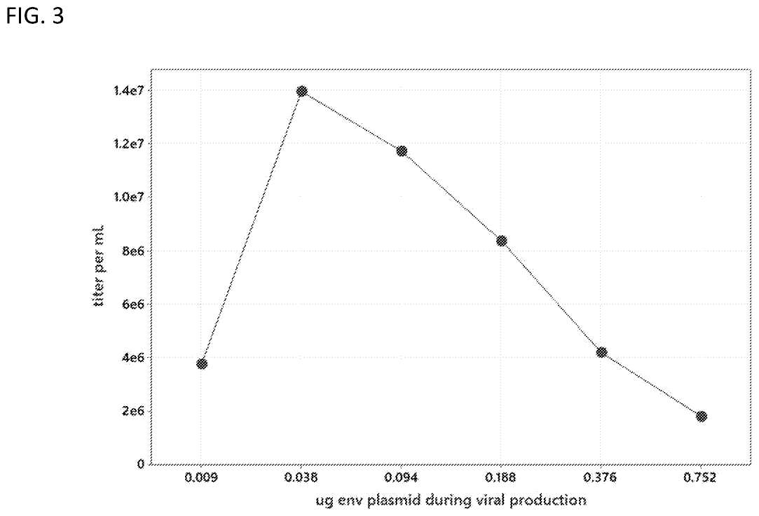

293T cells were transfected with plasmids similar to Example 1, with the difference that the plasmid encoding the exemplary BaEV envelope glycoprotein R+8 was transfected at different concentration ranges, which included 0.009, 0.038, 0.094, 0.188, 0.376, and 0.752 μg. Results for titer are shown in FIG. 3 which demonstrates a dose-dependent impact of plasmid concentration of viral titer.

### Example 3 Pseudotyped Lentivirus Activity on CD34+ Cells

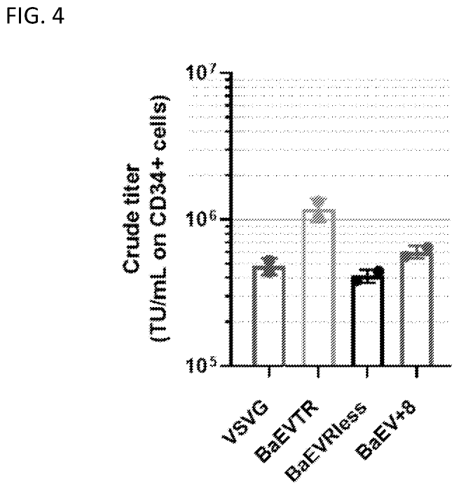

Lentiviral vectors were produced as described above pseudotyped with BaEV+8, BaEVTR, BaEVRless, or VSV-G. Transduction efficiency of crude production supernatants was tested on human CD34+ cells. Human CD34+ cells (Hemacare) from two donors were incubated for 24 hours in 6-well plates in serum-free medium supplemented with human recombinant cytokines cKIT, TPO, Flt3L (StemCell Tech). 2×10⁴ prestimulated CD34+ cells were transduced in retronectin-coated 96-well plates with crude LVs at various dilutions in serum-free medium. Cells were replenished with cytokines every 3 days. Eight days after transduction, the percentage of GFP+ cells was determined by flow cytometry and crude titer was estimated as shown in FIG. 4. The estimated crude titer of BaEV+8 was greater than that of VSV-G or BaEVRless. This example demonstrates that lentiviral vectors pseudotyped with BaEV+8 can transduce primary human CD34+ cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

VIII. SEQUENCES

| SEQ ID | | |
|---|---|---|
| NO | SEQUENCE | DESCRIPTION |
| 1 | MGFTTKIIFLYNLVLVYA | Signal sequence |
| 2 | GFDDPRKAIELVQKRYGRPCDCSGGQVSEPPSDRVSQ | Extracellular |
| | VTCSGKTAYLMPDQRWKCKSIPKDTSPSGPLQECPCN | domain |
| | SYQSSVHSSCYTSYQQCRSGNKTYYTATLLKTQTGGT | |
| | SDVQVLGSTNKLIQSPCNGIKGQSICWSTTAPIHVSD | |
| | GGGPLDTTRIKSVQRKLEEIHKALYPELQYHPLAIPK | |
| | VRDNLMVDAQTLNILNATYNLLLMSNTSLVDDCWLCL | |
| | KLGPPTPLAIPNFLLSYVTRSSDNISCLIIPPLLVQP | |
| | MQFSNSSCLFSPSYNSTEEIDLGHVAFSNCTSITNVT | |
| | GPICAVNGSVFLCGNNMAYTYLPTNWTGLCVLATLLP | |
| | DIDIIPGDEPVPIPAIDHFIYRPKRAIQFIPLLAGLG | |
| | ITAAFTTGATGLGVSVTQYTKLSNQLISDVQILSSTI | |
| | QDLQDQVDSLAEVVLQNRRGLDLLTAEQGGICLALQE | |
| | KCCFYVNKSGIVRDKIKTLQEELERRRKDLASNPLWT | |
| | GLQGLLP | |
| 3 | YLLPFLGPLLTLLLLLTIGPCIF | Transmembrane |
| | | domain |
| 4 | NRLTAFINDKLNIIHAMVLTQQYQVLRTDEEAQD | Full cytoplasmic |
| | | domain |
| | | including R |
| | | peptide |
| 5 | NRLTAFINDKLNIIHAM | Cytoplasmic tail |
| | | (R-less) |
| 6 | NRLTAFINDKLNIIHAMV | Cytoplasmic tail |
| | | (R + 1) |
| 7 | NRLTAFINDKLNIIHAMVL | Cytoplasmic tail |
| | | (R + 2) |
| 8 | NRLTAFINDKLNIIHAMVLT | Cytoplasmic tail |
| | | (R + 3) |
| 9 | NRLTAFINDKLNIIHAMVLTQ | Cytoplasmic tail |
| | | (R + 4) |
| 10 | NRLTAFINDKLNIIHAMVLTQQ | Cytoplasmic tail |
| | | (R + 5) |
| 11 | NRLTAFINDKLNIIHAMVLTQQY | Cytoplasmic tail |
| | | (R + 6) |
| 12 | NRLTAFINDKLNIIHAMVLTQQYQ | Cytoplasmic tail |
| | | (R + 7) |
| 13 | NRLTAFINDKLNIIHAMVLTQQYQV | Cytoplasmic tail |
| | | (R + 8) |
| 14 | NRLTAFINDKLNIIHAMVLTQQYQVL | Cytoplasmic tail |
| | | (R + 9) |
| 15 | NRLTAFINDKLNIIHAMVLTQQYQVLR | Cytoplasmic tail |
| | | (R + 10) |
| 16 | NRLTAFINDKLNIIHAMVLTQQYQVLRT | Cytoplasmic tail |
| | | (R + 11) |
| 17 | NRLTAFINDKLNIIHAMVLTQQYQVLRTD | Cytoplasmic tail |
| | | (R + 12) |
| 18 | NRLTAFINDKLNIIHAMVLTQQYQVLRTDE | Cytoplasmic tail |
| | | (R + 13) |
| 19 | NRLTAFINDKLNIIHAMVLTQQYQVLRTDEE | Cytoplasmic tail |
| | | (R + 14) |
| 20 | NRLTAFINDKLNIIHAMVLTQQYQVLRTDEEA | Cytoplasmic tail |
| | | (R + 15) |
| 21 | NRLTAFINDKLNIIHAMVLTQQYQVLRTDEEAQ | Cytoplasmic tail |
| | | (R + 16) |
| 22 | VLTQQYQVLRTDEEAQD | Full R peptide |
| | | only |
| 23 | MGFTTKIIFLYNLVLVYAGFDDPRKAIELVQKRYGR | Full Protein with |
| | PCDCSGGQVSEPPSDRVSQVTCSGKTAYLMPDQRWK | signal sequence |
| | CKSIPKDTSPSGPLQECPCNSYQSSVHSSCYTSYQQ | |
| | CRSGNKTYYTATLLKTQTGGTSDVQVLGSTNKLIQS | |
| | PCNGIKGQSICWSTTAPIHVSDGGGPLDTTRIKSVQ | |
| | RKLEEIHKALYPELQYHPLAIPKVRDNLMVDAQTLN | |
| | ILNATYNLLLMSNTSLVDDCWLCLKLGPPTPLAIPN | |
| | FLLSYVTRSSDNISCLIIPPLLVQPMQFSNSSCLFS | |
| | PSYNSTEEIDLGHVAFSNCTSITNVTGPICAVNGSV | |
| | FLCGNNMAYTYLPTNWTGLCVLATLLPDIDIIPGDE | |
| | PVPIPAIDHFIYRPKRAIQFIPLLAGLGITAAFTTG | |
| | ATGLGVSVTQYTKLSNQLISDVQILSSTIQDLQDQV | |
| | DSLAEVVLQNRRGLDLLTAEQGGICLALQEKCCFYV | |
| | NKSGIVRDKIKTLQEELERRRKDLASNPLWTGLQGL | |
| | LPYLLPFLGPLLTLLLLLTIGPCIFNRLTAFINDKL | |
| | NIIHAMVLTQQYQVLRTDEEAQD | |
| 24 | GFDDPRKAIELVQKRYGRPCDCSGGQVSEPPSDRVS | Full protein |
| | QVTCSGKTAYLMPDQRWKCKSIPKDTSPSGPLQECP | without signal |
| | CNSYQSSVHSSCYTSYQQCRSGNKTYYTATLLKTQT | sequence |
| | GGTSDVQVLGSTNKLIQSPCNGIKGQSICWSTTAPI | (cytoplasmic tail, |
| | HVSDGGGPLDTTRIKSVQRKLEEIHKALYPELQYHP | bold and |
| | LAIPKVRDNLMVDAQTLNILNATYNLLLMSNTSLVD | underline) |
| | DCWLCLKLGPPTPLAIPNFLLSYVTRSSDNISCLII | |
| | PPLLVQPMQFSNSSCLFSPSYNSTEEIDLGHVAFSN | |
| | CTSITNVTGPICAVNGSVFLCGNNMAYTYLPTNWTG | |
| | LCVLATLLPDIDIIPGDEPVPIPAIDHFIYRPKRAI | |
| | QFIPLLAGLGITAAFTTGATGLGVSVTQYTKLSNQL | |
| | ISDVQILSSTIQDLQDQVDSLAEVVLQNRRGLDLLT | |
| | AEQGGICLALQEKCCFYVNKSGIVRDKIKTLQEELE | |
| | RRRKDLASNPLWTGLQGLLPYLLPFLGPLLTLLLLL | |
| | TIGPCIFNRLTAFINDKLNIIHAMVLTQQYQVLRTD | |
| | EEAQD | |
| 25 | GFDDPRKAIELVQKRYGRPCDCSGGQVSEPPSDRVSQ | Surface protein |
| | VTCSGKTAYLMPDQRWKCKSIPKDTSPSGPLQECPC | subunit |
| | NSYQSSVHSSCYTSYQQCRSGNKTYYTATLLKTQTG | |
| | GTSDVQVLGSTNKLIQSPCNGIKGQSICWSTTAPIH | |
| | VSDGGGPLDTTRIKSVQRKLEEIHKALYPELQYHPL | |
| | AIPKVRDNLMVDAQTLNILNATYNLLLMSNTSLVDD | |
| | CWLCLKLGPPTPLAIPNFLLSYVTRSSDNISCLIIP | |
| | PLLVQPMQFSNSSCLFSPSYNSTEEIDLGHVAFSNC | |
| | TSITNVTGPICAVNGSVFLCGNNMAYTYLPTNWTGL | |
| | CVLATLLPDIDIIPGDEPVPIPAIDHFIYRPKR | |
| 26 | AIQFIPLLAGLGITAAFTTGATGLGVSVTQYTKLSNQ | Transmembrane |
| | LISDVQILSSTIQDLQDQVDSLAEVVLQNRRGLDLLT | protein subunit |
| | AEQGGICLALQEKCCFYVNKSGIVRDKIKTLQEELER | (without R |
| | RRKDLASNPLWTGLQGLLPYLLPFLGPLLTLLLLLTI | peptide) |
| | GPCIFNRLTAFINDKLNIIHAM | |
| 27 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (including R |
| | RKDLASNPLWTGLQGLLPYLLPFLGPLLTLLLLLTIGP | peptide) |
| | CIF NRLTAFINDKLNIIHAMVLTQQYQVLRTDEEAQD | |
| 28 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein R- |
| | NSYQSSVHSSCYTSYQQCRSGNKTYYTATLLKTQTGG | less |
| | TSDVQVLGSTNKLIQSPCNGIKGQSICWSTTAPIHVS | |
| | DGGGPLDTTRIKSVQRKLEEIHKALYPELQYHPLAIP | |
| | KVRDNLMVDAQTLNILNATYNLLLMSNTSLVDDCWLC | |
| | LKLGPPTPLAIPNFLLSYVTRSSDNISCLIIPPLLVQ | |
| | PMQFSNSSCLFSPSYNSTEEIDLGHVAFSNCTSITNV | |
| | TGPICAVNGSVFLCGNNMAYTYLPTNWTGLCVLATLL | |
| | PDIDIIPGDEPVPIPAIDHFIYRPKRAIQFIPLLAGL | |
| | GITAAFTTGATGLGVSVTQYTKLSNQLISDVQILSST | |
| | IQDLQDQVDSLAEVVLQNRRGLDLLTAEQGGICLALQ | |
| | EKCCFYVNKSGIVRDKIKTLQEELERRRKDLASNPLW | |
| | TGLQGLLPYLLPFLGPLLTLLLLLTIGPCIFNRLTAF | |
| | INDKLNIIHAM | |
| 29 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 1 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMV | |
| 30 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 2 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVL | |
| 31 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 3 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLT | |
| 32 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 4 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQ | |
| 33 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 5 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQ | |
| 34 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 6 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQY | |
| 35 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 7 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQ | |
| 36 | GFDDPRKAIELVQKRYGRPCDCSGGQVSEPPSDRVS | BaEv envelope |
| | QVTCSGKTAYLMPDQRWKCKSIPKDTSPSGPLQECP | glycoprotein |
| | CNSYQSSVHSSCYTSYQQCRSGNKTYYTATLLKTQT | R + 8 |
| | GGTSDVQVLGSTNKLIQSPCNGIKGQSICWSTTAPI | |
| | HVSDGGGPLDTTRIKSVQRKLEEIHKALYPELQYHP | |
| | LAIPKVRDNLMVDAQTLNILNATYNLLLMSNTSLVD | |
| | DCWLCLKLGPPTPLAIPNFLLSYVTRSSDNISCLII | |
| | PPLLVQPMQFSNSSCLFSPSYNSTEEIDLGHVAFSN | |
| | CTSITNVTGPICAVNGSVFLCGNNMAYTYLPTNWTG | |
| | LCVLATLLPDIDIIPGDEPVPIPAIDHFIYRPKRAI | |
| | QFIPLLAGLGITAAFTTGATGLGVSVTQYTKLSNQL | |
| | ISDVQILSSTIQDLQDQVDSLAEVVLQNRRGLDLLT | |
| | AEQGGICLALQEKCCFYVNKSGIVRDKIKTLQEELE | |
| | RRRKDLASNPLWTGLQGLLPYLLPFLGPLLTLLLLL | |
| | TIGPCIFNRLTAFINDKLNIIHAMVLTQQYQV | |
| 37 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 9 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVL | |
| 38 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 10 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLR | |
| 39 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 11 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRT | |
| 40 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 12 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRTD | |
| 41 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 13 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRTDE | |
| 42 | GFDDPRKAIELVQKRYGRPCDCSGGQVSEPPSDRVSQ | |
| | VTCSGKTAYLMPDQRWKCKSIPKDTSPSGPLQECPC | BaEv envelope |
| | NSYQSSVHSSCYTSYQQCRSGNKTYYTATLLKTQTG | glycoprotein |
| | GTSDVQVLGSTNKLIQSPCNGIKGQSICWSTTAPIHV | R + 14 |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRTDEE | |
| 43 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 15 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRTDEEA | |
| 44 | GFDDPRKAIELVQKRYGRPCDCSGGQVSEPPSDRVSQ | BaEv envelope |
| | VTCSGKTAYLMPDQRWKCKSIPKDTSPSGPLQECPC | glycoprotein |
| | NSYQSSVHSSCYTSYQQCRSGNKTYYTATLLKTQTG | R + 16 |
| | GTSDVQVLGSTNKLIQSPCNGIKGQSICWSTTAPIHV | |
| | SDGGGPLDTTRIKSVQRKLEEIHKALYPELQYHPLAI | |
| | PKVRDNLMVDAQTLNILNATYNLLLMSNTSLVDDCWL | |
| | CLKLGPPTPLAIPNFLLSYVTRSSDNISCLIIPPLLV | |
| | QPMQFSNSSCLFSPSYNSTEEIDLGHVAFSNCTSITN | |
| | VTGPICAVNGSVFLCGNNMAYTYLPTNWTGLCVLATL | |
| | LPDIDIIPGDEPVPIPAIDHFIYRPKRAIQFIPLLAG | |
| | LGITAAFTTGATGLGVSVTQYTKLSNQLISDVQILSS | |
| | TIQDLQDQVDSLAEVVLQNRRGLDLLTAEQGGICLAL | |
| | QEKCCFYVNKSGIVRDKIKTLQEELERRRKDLASNPL | |
| | WTGLQGLLPYLLPFLGPLLTLLLLLTIGPCIFNRLTA | |
| | FINDKLNIIHAMVLTQQYQVLRTDEEAQ | |
| 45 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 1 peptide) |
| | RKDLASNPLWTGLQGLLPYLLPFLGPLLTLLLLLTIGP | |
| | CIF NRLTAFINDKLNIIHAMV | |
| 46 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 2 peptide) |
| | RKDLASNPLWTGLQGLLPYLLPFLGPLLTLLLLLTIGP | |
| | CIF NRLTAFINDKLNIIHAMVL | |
| 47 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 3 peptide) |
| | RKDLASNPLWTGLQGLLPYLLPFLGPLLTLLLLLTIGP | |
| | CIF NRLTAFINDKLNIIHAMVLT | |
| 48 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 4 peptide) |
| | RKDLASNPLWTGLQGLLPYLLPFLGPLLTLLLLLTIGP | |
| | CIF NRLTAFINDKLNIIHAMVLTQ | |
| 49 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 5 peptide) |
| | RKDLASNPLWTGLQGLLPYLLPFLGPLLTLLLLLTIGP | |
| | CIF NRLTAFINDKLNIIHAMVLTQQ | |
| 50 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 6 peptide) |
| | RKDLASNPLWTGLQGLLPYLLPFLGPLLTLLLLLTIGP | |
| | CIF NRLTAFINDKLNIIHAMVLTQQY | |
| 51 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 7 peptide) |
| | RKDLASNPLWTGLQGLLPYLLPFLGPLLTLLLLLTIGP | |
| | CIF NRLTAFINDKLNIIHAMVLTQQYQ | |
| 52 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 8 peptide) |
| | RKDLASNPLWTGLQGLLPYLLPFLGPLLTLLLLLTIGP | |
| | CIF NRLTAFINDKLNIIHAMVLTQQYQV | |
| 53 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 9 peptide) |
| | RKDLASNPLWTGLQGLLPYLLPFLGPLLTLLLLLTIGP | |
| | CIF NRLTAFINDKLNIIHAMVLTQQYQVL | |
| 54 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 10 peptide) |
| | RKDLASNPLWTGLQGLLPYLLPFLGPLLTLLLLLTIGP | |
| | CIF NRLTAFINDKLNIIHAMVLTQQYQVLR | |
| 55 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 11 peptide) |
| | RKDLASNPLWTGLQGLLPYLLPFLGPLLTLLLLLTIGP | |
| | CIF NRLTAFINDKLNIIHAMVLTQQYQVLRT | |
| 56 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 12 peptide) |
| | RKDLASNPLWTGLQGLLPYLLPFLGPLLTLLLLLTIGP | |
| | CIF NRLTAFINDKLNIIHAMVLTQQYQVLRTD | |
| 57 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 13 peptide) |
| | RKDLASNPLWTGLQGLLPYLLPFLGPLLTLLLLLTIGP | |
| | CIF NRLTAFINDKLNIIHAMVLTQQYQVLRTDE | |
| 58 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 14 peptide) |
| | RKDLASNPLWTGLQGLLPYLLPFLGPLLTLLLLLTIGP | |
| | CIF NRLTAFINDKLNIIHAMVLTQQYQVLRTDEE | |
| 59 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 15 peptide) |
| | RKDLASNPLWTGLQGLLPYLLPFLGPLLTLLLLLTIGP | |
| | CIF NRLTAFINDKLNIIHAMVLTQQYQVLRTDEEA | |
| 60 | AIQFIPLLAGLGITAAFTTGATGLGVSVTQYTKLSNQL | Transmembrane |
| | ISDVQILSSTIQDLQDQVDSLAEVVLQNRRGLDLLTA | protein subunit |
| | EQGGICLALQEKCCFYVNKSGIVRDKIKTLQEELERR | (R + 16 peptide) |
| | RKDLASNPLWTGLQGLLPYLLPFLGPLLTLLLLLTIGP | |
| | CIF NRLTAFINDKLNIIHAMVLTQQYQVLRTDEEAQ | |
| 61 | DIQMTQTTSSLSASLGDRVTISCRASQDISKYLNWYQ | Anti-CD19 scFv |
| | QKPDGTVKLLIYHTSRLHSGVPSRFSGSGSGTDYSLTI | (FMC63) |
| | SNLEQEDIATYFCQQGNTLPYTFGGGTKLEITGSTSGS | |
| | GKPGSGEGSTKGEVKLQESGPGLVAPSQSLSVTCTVS | |
| | GVSLPDYGVSWIRQPPRKGLEWLGVIWGSETTYYNS | |
| | ALKSRLTIIKDNSKSQVFLKMNSLQTDDTAIYYCAKH | |
| | YYYGGSYAMDYWGQGTSVTVSS | |
| 62 | DIQMTQTTSSLSASLGDRVTISCRASQDISKYLNWYQ | Anti-CD19 scFv |
| | QKPDGTVKLLIYHTSRLHSGVPSRFSGSGSGTDYSLTI | (FMC63) |
| | SNLEQEDIATYFCQQGNTLPYTFGGGTKLEITGGGGS | |
| | GGGGSGGGGSEVKLQESGPGLVAPSQSLSVTCTVSG | |
| | VSLPDYGVSWIRQPPRKGLEWLGVIWGSETTYYNSA | |
| | LKSRLTIIKDNSKSQVFLKMNSLQTDDTAIYYCAKHY | |
| | YYGGSYAMDYWGQGTSVTVSS | |
| 63 | ESKYGPPCPPCP | IgG4 Hinge |
| 64 | TTTPAPRPPTPAPTIASQPLSLRPE | CD8 Hinge |
| 65 | IEVMYPPPYLDNEKSNGTIIHVKGKHLCPSPLFPGPSK | CD28 |
| | P | |
| 66 | ACRPAAGGAVHTRGLDFACDIYIWAPLAGTCGVLLL | CD8 |
| | SLVITLYC | |
| 67 | FWVLVVVGGVLACYSLLVTVAFIIFWV | CD28 |
| 68 | FWVLVVVGGVLACYSLLVTVAFIIFWV | CD28 |
| 69 | RSKRSRLLHSDYMNMTPRRPGPTRKHYQPYAPPRDF | CD28 |
| | AAYRS | |
| 70 | KRGRKKLLYIFKQPFMRPVQTTQEEDGCSCRFPEEEE | 4-1BB |
| | GGCEL | |
| 71 | RVKFSRSADAPAYQQGQNQLYNELNLGRREEYDVL | CD3zeta |
| | DKRRGRDPEMGGKPRRKNPQEGLYNELQKDKMAEA | |
| | YSEIGMKGERRRGKGHDGLYQGLSTATKDTYDALH | |
| | MQALPPR | |
| 72 | RVKFSRSADAPAYKQGQNQLYNELNLGRREEYDVL | CD3zeta |
| | DKRRGRDPEMGGKPRRKNPQEGLYNELQKDKMAEA | |
| | YSEIGMKGERRRGKGHDGLYQGLSTATKDTYDALH | |
| | MQALPPR | |

What is claimed:

1. A Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one amino-terminal amino acid but less than the full length of the inhibitory R peptide of the wild-type BaEv envelope glycoprotein.

2. A Baboon Endogenous Retrovirus (BaEV) envelope glycoprotein pseudotyped lentiviral particle, comprising a truncated BaEV envelope glycoprotein comprising a cytoplasmic tail with a partial fusion inhibitory R peptide relative to a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein.

3. The lentiviral particle of claim 1 or claim 2 that is replication defective.

4. The lentiviral particle of any of claims 1-3 prepared by a method comprising transducing a producer cell with packaging plasmids that encode a Gag-pol, Rev, Tat and the truncated BaEV envelope glycoprotein.

5. The lentiviral particle of any of claims 1-4, wherein the lentiviral particle further comprises a viral nucleic acid.

6. The lentiviral particle of claim 5, wherein the viral nucleic acid comprises one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

7. The lentiviral particle of any of claims 1-4, wherein the lentiviral particle is devoid of viral genomic DNA.

8. A lipid particle comprising a truncated Baboon Endogenous Retrovirus (BaEV) envelope glycoprotein, comprising:
(a) a lipid bilayer enclosing a lumen, and
(b) a truncated BaEV envelope glycoprotein comprising a cytoplasmic tail with a partial fusion inhibitory R peptide relative to the inhibitory R peptide of a wild-type BaEV envelope glycoprotein, wherein the partial fusion inhibitory R peptide comprises at least one contiguous amino-terminal amino acids but less than the full length of the inhibitory R peptide of the inhibitory R peptide of the wild-type BaEV envelope glycoprotein, wherein the envelope glycoprotein is embedded in the lipid bilayer.

9. A lipid particle comprising a truncated Baboon Endogenous Retrovirus (BaEV) envelope glycoprotein, comprising:
(a) a lipid bilayer enclosing a lumen, and
(b) a truncated BaEV envelope glycoprotein comprising a cytoplasmic tail with a partial fusion inhibitory R peptide relative to the inhibitory R peptide of a wild-type BaEV envelope glycoprotein, wherein the cytoplasmic tail is 25 amino acids in length and contains 8 contiguous amino-terminal acids of the inhibitory R peptide (R+8) of the full length inhibitory R peptide of wild-type BaEV envelope glycoprotein, wherein the envelope glycoprotein is embedded in the lipid bilayer.

10. The lipid particle of claim 8 or claim 9, wherein the lipid bilayer is derived from a membrane of a host cell used for producing a retrovirus or retrovirus-like particle.

11. The lipid particle of claim 10, wherein the host cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

12. The lipid particle of any of claims 8-11, wherein the lipid bilayer is or comprises one or more other viral components other than the BaEV envelope glycoprotein.

13. The lipid particle of claim 12, wherein the one or more viral components are from a retrovirus.

14. The lipid particle of 10-13, wherein the retrovirus is a lentivirus or a lentivirus like particle.

15. The lentiviral particle of any of claims 1-7 or the lipid particle of an of claims 8-14, wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

16. The lentiviral particle or lipid particle of claim 15, wherein the glycoprotein 70 (g70) subunit or a biologically active portion thereof, and the portion of the glycoprotein p20E (p20E) subunit are associated via an inter-subunit disulfide bond.

17. The lentiviral particle of lipid particle of any of claims 1-16, wherein the BaEV glycoprotein binds an ASCT-2 or ASCT-1 receptor.

18. The lentiviral particle or lipid particle of any of claims 15-17, wherein the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25.

19. The lentiviral particle or lipid particle of any of claims 15-18, wherein the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

20. The lentiviral particle or lipid particle of any of claims 1-19, wherein the truncated BaEV envelope glycoprotein is truncated relative to SEQ ID NO:24 and lacks up to 16 contiguous amino acids from the C-terminal cytoplasmic tail of SEQ ID NO:24.

21. The lentiviral particle or lipid particle of any of claims 1-20, wherein the truncated BaEV envelope glycoprotein is truncated relative to SEQ ID NO:24 and lacks from 8 to 14, contiguous amino acids from the C-terminal cytoplasmic tail of SEQ ID NO:24, optionally from 8 to 13 contiguous amino acids from the C-terminal cytoplasmic tail of SEQ ID NO:24.

22. The lentiviral particle or lipid particle of any of claims 1, 3-7, 8 and 10-19, wherein the partial fusion inhibitory R peptide is set forth as amino acids 1 to 9 of SEQ ID NO:22, optionally wherein the cytoplasmic tail is set forth in SEQ ID NO:14 (R+9).

23. The lentiviral particle or lipid particle of any of claims 1, 3-7, 8 and 10-22, wherein the truncated BaEV envelope glycoprotein is set forth in SEQ ID NO: 37.

24. The lentiviral particle or lipid particle of any of claims 1-19, wherein the partial fusion inhibitory R peptide is set forth as amino acids 1 to 8 of SEQ ID NO:22, optionally wherein the cytoplasmic tail is set forth in SEQ ID NO:13 (R+8).

25. The lentiviral particle or lipid particle of any of claims 1-19 and 24, wherein the truncated BaEV envelope glycoprotein is set forth in SEQ ID NO: 36.

26. The lentiviral particle or lipid particle of any of claims 1, 3-7, 8 and 10-19, wherein the partial fusion inhibitory R peptide is set forth as amino acids 1 to 7 of SEQ ID NO:22, optionally wherein the cytoplasmic tail is set forth in SEQ ID NO: 12 (R+7).

27. The lentiviral particle or lipid particle of any of claims 1, 3-7, 8, 10-19 and 26, wherein the truncated BaEV envelope glycoprotein is set forth in SEQ ID NO: 35.

28. The lentiviral particle or lipid particle of any of claims 1, 3-7, 8, and 10-19, wherein the partial fusion inhibitory R peptide is set forth as amino acids 1 to 6 of SEQ ID NO:22, optionally wherein the cytoplasmic tail is set forth in SEQ ID NO:11 (R+6).

29. The lentiviral particle or lipid particle of any of claims 1, 3-7, 8, 10-19 and 28, wherein the truncated BaEV envelope glycoprotein is set forth in SEQ ID NO: 34.

30. The lentiviral particle or lipid particle of any of claims 1, 3-7, 8, and 10-19, wherein the partial fusion inhibitory R peptide is set forth as amino acids 1 to 5 of SEQ ID NO:22, optionally wherein the cytoplasmic tail is set forth in SEQ ID NO: 10 (R+5).

31. The lentiviral particle or lipid particle of any of claims 1, 3-7, 8, 10-19 and 30, wherein the truncated BaEV envelope glycoprotein is set forth in SEQ ID NO: 33.

32. The lentiviral particle or lipid particle of any of claims 1, 3-7, 8, and 10-19, wherein the partial fusion inhibitory R peptide is set forth as amino acids 1 to 4 of SEQ ID NO:22, optionally wherein the cytoplasmic tail is set forth in SEQ ID NO:9 (R+4).

33. The lentiviral particle or lipid particle of any of claims 1, 3-7, 8, 10-19 and 32, wherein the truncated BaEV envelope glycoprotein is set forth in SEQ ID NO: 32.

34. The lentiviral particle or lipid particle of any of claims 1, 3-7, 8, and 10-19, wherein the partial fusion inhibitory R peptide is set forth as amino acids 1 to 3 of SEQ ID NO:22, optionally wherein the cytoplasmic tail is set forth in SEQ ID NO:8 (R+3).

35. The lentiviral particle or lipid particle of any of claims 1, 3-7, 8, 10-19 and 34, wherein the truncated BaEV envelope glycoprotein is set forth in SEQ ID NO: 31.

36. The lentiviral particle or lipid particle of any of claims 1-35, wherein the particle further comprises an exogenous agent.

37. The lentiviral particle or lipid particle of claim 36, wherein the exogenous agent is present in the lumen.

38. The lentiviral particle or lipid particle of claim 36 or claim 37, wherein the exogenous agent is a protein or a nucleic acid, optionally wherein the nucleic acid is a DNA or RNA.

39. The lentiviral particle of any of claims 36-38, wherein the exogenous agent is or encodes a factor associated with gene editing.

40. The lentiviral particle of any of claims 36-39, wherein the exogenous agent is or encodes a factor associated with base editing and/or prime editing (i.e, target-primed reverse transcription (TPRT)).

41. The lentiviral particle of any of claims 36-40, wherein the exogenous agent is or encodes a nuclease, optionally wherein the nuclease is selected from the group comprising a zinc-finger nucleases (ZFNs), transcription-activator like effector nucleases (TALENs), or a CRISPR-associated protein-nuclease (Cas)

42. The lentiviral particle of any of claims 36-40, wherein the exogenous agent is or encodes a transposase and/or recombinase.

43. The lentiviral particle of any of claims 36-40, wherein the exogenous agent is or encodes a DNA polymerase, RNA polymerase, or reverse-transcriptase.

44. The lentiviral particle or lipid particle of any of claims 1-43, wherein the particle is produced as a preparation with increased titer compared to a reference particle preparation that is similarly produced but with incorporation of a BaEV envelope glycoprotein having a cytoplasmic tail with a full length R peptide or a portion of the R peptide of 10 or more amino acids in length.

45. The lentiviral particle or lipid particle of claim 44, wherein the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more.

46. The lentiviral particle or lipid of any of claims 1-45, wherein the titer in target cells, optionally HEK293 cells, following transduction is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL.

47. The lentiviral particle or lipid particle of any of claims 1-46, wherein the truncated BaEV envelope glycoprotein is present on the surface of the particle at a density of at least about (0.001, 0.002, 0.005, 0.01, 0.02, 0.05, 0.1, 0.2 or 0.5) truncated BaEV envelope glycoproteins/nm².

48. A truncated BaEV envelope glycoprotein comprising a cytoplasmic tail with a partial fusion inhibitory R peptide relative to a wild-type BaEV envelope glycoprotein, wherein the partial fusion inhibitory R peptide comprises at least one contiguous amino-terminal amino acid, but less than the full length, of the inhibitory R peptide of the wild-type BaEV envelope glycoprotein.

49. The truncated BaEV envelope glycoprotein of claim 48 wherein the truncated BaEV glycoprotein comprises: (i) a glycoprotein 70 (g70) subunit or a biologically active portion thereof, and (ii) a portion of the glycoprotein p20E (p20E) subunit comprising the cytoplasmic tail with the partial inhibitory R peptide.

50. The truncated BaEV envelope glycoprotein of claim 49, wherein the glycoprotein 70 (g70) subunit or a biologically active portion thereof, and the portion of the glycoprotein p20E (p20E) subunit are associated via an inter-subunit disulfide bond.

51. The truncated BaEV envelope glycoprotein of any of claims 48-50, wherein the BaEV glycoprotein binds an ASCT-2 or ASCT-1 receptor.

52. The truncated BaEV glycoprotein of any of claims 48-51, wherein the glycoprotein 70 (g70) subunit or a biologically active portion thereof comprises the amino acid sequence set forth in SEQ ID NO:25, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to the amino acid sequence set forth in SEQ ID NO:25.

53. The truncated BaEV glycoprotein of any of claims 48-52, wherein the portion of the glycoprotein p20E (p20E) subunit comprises SEQ ID NO:26, or a sequence that exhibits at least at least at or about 80%, at least at or about 81%, at least at or about 82%, at least at or about 83%, at or about 84%, at least at or about 85%, at least at or about 86%, or at least at or about 87%, at least at or about 88%, or at least at or about 89%, at least at or about 90%, at least at or about 91%, at least at or about 92%, at least at or about 93%, at least at or about 94%, at least at or about 95%, at or about 96%, at least at or about 97%, at least at or about 98%, or at least at or about 99% sequence identity to SEQ ID NO:26, and comprises the partial inhibitory R peptide.

54. The truncated BaEV envelope glycoprotein of any of claims 48-53, wherein the truncated BaEV envelope glycoprotein is truncated relative to SEQ ID NO:24 and lacks up to 16 contiguous amino acids from the C-terminal cytoplasmic tail of SEQ ID NO:24.

55. The truncated BaEV envelope glycoprotein of any of claims 48-54, wherein the truncated BaEV envelope glycoprotein is truncated relative to SEQ ID NO:24 and lacks from 8 to 14 contiguous amino acids from the C-terminal cytoplasmic tail of SEQ ID NO:24, optionally from 8 to 13 contiguous amino acids from the C-terminal cytoplasmic tail of SEQ ID NO:24.

56. The truncated BaEV envelope glycoprotein of any of claims 48-55, wherein the partial fusion inhibitory R peptide is set forth as amino acids 1 to 9 of SEQ ID NO:22, optionally wherein the cytoplasmic tail is set forth in SEQ ID NO:14 (R+9).

57. The truncated BaEV envelope glycoprotein of any of claims 48-56, wherein the truncated BaEV envelope glycoprotein is set forth in SEQ ID NO: 37.

58. The truncated BaEV envelope glycoprotein of any of claims 48-55, wherein the partial fusion inhibitory R peptide is set forth as amino acids 1 to 8 of SEQ ID NO:22, optionally wherein the cytoplasmic tail is set forth in SEQ ID NO:13 (R+8).

59. The truncated BaEV envelope glycoprotein of any of claims 48-55 and 58, wherein the truncated BaEV envelope glycoprotein is set forth in SEQ ID NO: 36.

60. The truncated BaEV envelope glycoprotein of any of claims 48-55, wherein the partial fusion inhibitory R peptide is set forth as amino acids 1 to 7 of SEQ ID NO:22, optionally wherein the cytoplasmic tail is set forth in SEQ ID NO: 12 (R+7).

61. The truncated BaEV envelope glycoprotein of any of claims 48-55 and 60, wherein the truncated BaEV envelope glycoprotein is set forth in SEQ ID NO: 35.

62. The truncated BaEV envelope glycoprotein of any of claims 48-55, wherein the partial fusion inhibitory R peptide is set forth as amino acids 1 to 6 of SEQ ID NO:22, optionally wherein the cytoplasmic tail is set forth in SEQ ID NO:11 (R+6).

63. The truncated BaEV envelope glycoprotein of any of claims 48-55 and 62, wherein the truncated BaEV envelope glycoprotein is set forth in SEQ ID NO: 34.

64. The truncated BaEV envelope glycoprotein of any of claims 48-55, wherein the partial fusion inhibitory R peptide is set forth as amino acids 1 to 5 of SEQ ID NO:22, optionally wherein the cytoplasmic tail is set forth in SEQ ID NO:10 (R+5).

65. The truncated BaEV envelope glycoprotein of any of claims 48-55 and 64, wherein the truncated BaEV envelope glycoprotein is set forth in SEQ ID NO: 33.

66. The truncated BaEV envelope glycoprotein of any of claims 48-55, wherein the partial fusion inhibitory R peptide is set forth as amino acids 1 to 4 of SEQ ID NO:22, optionally wherein the cytoplasmic tail is set forth in SEQ ID NO:9 (R+4).

67. The truncated BaEV envelope glycoprotein of any of claims 48-55 and 66, wherein the truncated BaEV envelope glycoprotein is set forth in SEQ ID NO: 32.

68. The truncated BaEV envelope glycoprotein of any of claims 48-55, wherein the partial fusion inhibitory R peptide is set forth as amino acids 1 to 3 of SEQ ID NO:22, optionally wherein the cytoplasmic tail is set forth in SEQ ID NO:8 (R+3).

69. The truncated BaEV envelope glycoprotein of any of claims 48-55 and 68, wherein the truncated BaEV envelope glycoprotein is set forth in SEQ ID NO: 31.

70. A polynucleotide comprising a nucleic acid encoding the truncated BaEV envelope glycoprotein of any of claims 48-69.

71. The polynucleotide of claim 70, wherein the polynucleotide is codon optimized.

72. The polynucleotide of claim 70 or claim 71, further comprising at least one promoter that is operatively linked to control expression of the nucleic acid.

73. The polynucleotide of claim 72, wherein the promoter is a constitutive promoter.

74. The polynucleotide of claim 72 or claim 73, wherein the promoter is an inducible promoter.

75. A vector, comprising the polynucleotide of any of claims 70-74.

76. A plasmid, comprising the polynucleotide of any of claims 70-74.

77. The plasmid of claim 76, further comprising one or more nucleic acids encoding proteins for lentivirus production.

78. A cell comprising the polynucleotide of any of claims 70-74, the vector of claim 75 or the plasmid of claim 76 or claim 77.

79. The cell of claim 78 that is a producer cell for production of a lentiviral particle.

80. A producer cell comprising (i) a viral nucleic acid(s) and (ii) a nucleic acid encoding the truncated BaEV envelope glycoprotein of any of claims 48-69, optionally wherein the viral nucleic acid(s) are lentiviral nucleic acids.

81. The producer cell of claim 80, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

82. The producer cell of claim 80 or claim 81, wherein the producer cell comprises 293T cells.

83. The producer cell of any of claims 80-82, wherein the viral nucleic acid(s) lacks one or more genes involved in viral replication.

84. The producer cell of any of claims 80-83, wherein the viral nucleic acid comprises a nucleic acid encoding a viral packaging protein selected from one or more of Gag, Pol, Rev and Tat.

85. The producer cell of any of claims 80-84, wherein the viral nucleic acid comprises: one or more of (e.g., all of) the following nucleic acid sequences: 5′ LTR (e.g., comprising U5 and lacking a functional U3 domain), Psi packaging element (Psi), Central polypurine tract (cPPT)/central termination sequence (CTS) (e.g. DNA flap), Poly A tail sequence, a posttranscriptional regulatory element (e.g. WPRE), a Rev response element (RRE), and 3′ LTR (e.g., comprising U5 and lacking a functional U3).

86. A method of making a lipid particle comprising a truncated BaEV glycoprotein comprising:
a) introducing into a source cell a polynucleotide of any of claims 70-74, the vector of claim 75, or the plasmid of claim 76 or claim 77;
b) culturing the cell under conditions that allow for production of a lipid particle, and
c) separating, enriching, or purifying the lipid particle from the cell, thereby making the lipid particle.

87. The method of claim 86, wherein the source cell is a mammalian cell.

88. The method of claim 86 or claim 87, wherein the source cell is a producer cell and the lipid particle is a viral particle or a viral-like particle, optionally a retroviral particle or a retroviral-like particle, optionally a lentiviral particle or lentiviral-like particle.

89. A method of making a pseudotyped lentiviral particle, the method comprising:
a) providing a producer cell that comprises a lentiviral nucleic acid (s) and a nucleic acid encoding a truncated BaEV envelope glycoprotein of any of claims 48-69 or the polynucleotide of any of claims 70-74;
b) culturing the cell under conditions that allow for production of the lentiviral particle, and
c) separating, enriching, or purifying the lentiviral particle from the producer cell, thereby making the pseudotyped lentiviral particle.

90. The method of claim 88 or claim 89, wherein the producer cell is selected from the group consisting of CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211A cells.

91. The method of any of claims 88-90, wherein the producer cell comprises 293T cells.

92. The method of any of claims 88-91, wherein the method produces a lentiviral preparation with increased titer compared to a reference lentiviral particle preparation that is similarly produced but is pseudotyped with a BaEV envelope glycoprotein having a cytoplasmic tail with a full length R peptide or a portion of the R peptide of 10 or more amino acids in length.

93. The method of claim 92, wherein the titer is increased by at or greater than 1.5-fold, 2-fold, 3-fold, 4-fold, 5-fold, 6-fold, 7-fold, 8-fold, 9-fold, 10-fold, 15-fold, 20-fold, 30-fold or more, optionally at or about or greater than 5-fold or more.

94. The method of any of claims 88-93, wherein the method produces a lentiviral preparation with a titer in target cells, optionally HEK293 cells, following transduction that is at or greater than 3×10⁶ TU/mL, at or greater than 4×10⁶ TU/mL, at or greater than 5×10⁶ TU/mL, at or greater than 6×10⁶ TU/mL, at or greater than 7×10⁶ TU/mL, at or greater than 8×10⁶ TU/mL, at or greater than 9×10⁶ TU/mL, at or greater than 1×10⁷ TU/mL, or at or greater than 1.2×10⁷ TU/mL.

95. The method of any of claims 88-94, wherein the method results in reduced syncytia formation of the producer cell compared to a similar method but that is for production of a reference lentiviral particle preparation pseudotyped with a BaEV envelope glycoprotein having a cytoplasmic tail with no R peptide (Rless) or an R peptide of 3 contiguous amino terminal amino acids or less in length relative to the wild-type BaEV envelope glycoprotein R peptide.

96. The method of any of claims 88-95, wherein the method produces a lentiviral preparation with high titer (e.g. greater than 4×10⁶ TU/mL, greater than 5×10⁶ TU/mL, greater than 6×10⁶ TU/mL, greater than 7×10⁶ TU/mL, greater than 8×10⁶ TU/mL, greater than 9×10⁶ TU/mL, greater than 1×10⁷ TU/mL, or greater than 1.2×10⁷ TU/mL) and minimal syncytia formation of the producer cell during the method of production.

97. A lipid particle produced by the method of any of claims 86-88 and 90-96.

98. A lentiviral particle produced by the method of any of claims 89-96.

99. A lipid particle comprising the truncated BaEV envelope glycoprotein of any of claims 48-69.

100. A lentiviral particle pseudotyped with a truncated BaEV envelope glycoprotein of any of claims 48-69.

101. A composition comprising a plurality of the lentiviral particles of any of claims 1-7, 15-47, 98 and 100.

102. A composition comprising a plurality of the lipid particles of any of claims 8-47, 97 and 99.

103. The composition of claim 101 or claim 102 further comprising a pharmaceutically acceptable excipient.

104. A method of transducing a cell, the method comprising contacting a cell with the lentiviral particle of any of claims 1-7, 15-47, 98 and 100 or the composition of claim 101 or claim 103.

105. The method of claim 104, wherein the lipid particle or lentiviral vector comprises an exogenous agent and the transduction introduces the exogenous agent into the cell.

106. A method of delivering an exogenous agent into a cell, the method comprising contacting a lentiviral particle or lipid particle of any of claims 1-47 and 97-100 or the composition of any of claims 101-103 with a cell.

107. The method of any of claims 104-106, wherein the contacting is in vitro or ex vivo.

108. The method of any of claims 104-106, wherein the contacting is in vivo in a subject.

109. A method of delivering an exogenous agent to a cell in a subject, the method comprising administering to the subject the lentiviral particle or lipid particle of any of claims 1-47 and 97-100 or the composition of any of claims 101-103.

110. The method of any of claims 104-109, wherein the cell is a hematopoietic lineage cell.

111. The method of any of claims 104-110, wherein the cell is selected from the group consisting of myeloid-lymphoid balanced hematopoietic lineage cells, myeloid-biased hematopoietic lineage cells, lymphoid-biased hematopoietic lineage cells, a platelet-biased hematopoietic lineage cells, a platelet-myeloid-biased hematopoietic lineage cells, a long-term repopulating hematopoietic lineage cells, an intermediate-term repopulating hematopoietic lineage cells, or a short-term repopulating hematopoietic lineage cells.

112. The method of any of claims 104-111, wherein the cell is selected from monocytes, macrophages, neutrophils, basophils, eosinophils, erythrocytes, megakaryocytes and platelets.

113. The method of any of claims 104-112, wherein the cell is selected from T cells, B cells, natural killer (NK) cells and innate lymphoid cells.

114. The method of any of claims 104-113, wherein the cell is a hematopoietic stem cell (HSC).

115. The method of claim 114, wherein the subject has received a hematopoietic stem cell transplant.

116. The method of any of claim 105-115, wherein the exogenous agent is a protein or a nucleic acid, optionally wherein the nucleic acid is a DNA or RNA.

117. The method of any of claims 105-116, wherein the exogenous agent is or encodes a therapeutic agent for treating a disease or condition in the subject.

118. The method of any of claims 105-116, wherein the exogenous agent is or encodes a membrane protein, optionally a chimeric antigen receptor, for targeting an antigen associated with a disease or condition in the subject.

119. The method of any of claims 105-117, wherein the exogenous agent is for use in gene therapy to correct a genetic deficiency or replaces a deficient or missing gene in the subject.

120. The method of any of claims 104-119, wherein the subject is a human subject.


SEQUENCE LISTING (72 sequences)

SEQ ID NO: 1 - protein, 18 aa, Artificial Sequence
  Signal sequence
MGFTTKIIFLYNLVLVYA

SEQ ID NO: 2 - protein, 488 aa, Artificial Sequence
  Extracellular domain
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLP

SEQ ID NO: 3 - protein, 23 aa, Artificial Sequence
  Transmembrane domain
YLLPFLGPLLTLLLLLTIGPCIF

SEQ ID NO: 4 - protein, 34 aa, Artificial Sequence
  Full cytoplasmic domain including R peptide
NRLTAFINDKLNIIHAMVLTQQYQVLRTDEEAQD

SEQ ID NO: 5 - protein, 17 aa, Artificial Sequence
  Cytoplasmic tail (R-less)
NRLTAFINDKLNIIHAM

SEQ ID NO: 6 - protein, 18 aa, Artificial Sequence
  Cytoplasmic tail (R+1)
NRLTAFINDKLNIIHAMV

SEQ ID NO: 7 - protein, 19 aa, Artificial Sequence
  Cytoplasmic tail (R+2)
NRLTAFINDKLNIIHAMVL

SEQ ID NO: 8 - protein, 20 aa, Artificial Sequence
  Cytoplasmic tail (R+3)
NRLTAFINDKLNIIHAMVLT

SEQ ID NO: 9 - protein, 21 aa, Artificial Sequence
  Cytoplasmic tail (R+4)
NRLTAFINDKLNIIHAMVLTQ

SEQ ID NO: 10 - protein, 22 aa, Artificial Sequence
  Cytoplasmic tail (R+5)
NRLTAFINDKLNIIHAMVLTQQ

SEQ ID NO: 11 - protein, 23 aa, Artificial Sequence
  Cytoplasmic tail (R+6)
NRLTAFINDKLNIIHAMVLTQQY

SEQ ID NO: 12 - protein, 24 aa, Artificial Sequence
  Cytoplasmic tail (R+7)
NRLTAFINDKLNIIHAMVLTQQYQ

SEQ ID NO: 13 - protein, 25 aa, Artificial Sequence
  Cytoplasmic tail (R+8)
NRLTAFINDKLNIIHAMVLTQQYQV

SEQ ID NO: 14 - protein, 26 aa, Artificial Sequence
  Cytoplasmic tail (R+9)
NRLTAFINDKLNIIHAMVLTQQYQVL

SEQ ID NO: 15 - protein, 27 aa, Artificial Sequence
  Cytoplasmic tail (R+10)
NRLTAFINDKLNIIHAMVLTQQYQVLR

SEQ ID NO: 16 - protein, 28 aa, Artificial Sequence
  Cytoplasmic tail (R+11)
NRLTAFINDKLNIIHAMVLTQQYQVLRT

SEQ ID NO: 17 - protein, 29 aa, Artificial Sequence
  Cytoplasmic tail (R+12)
NRLTAFINDKLNIIHAMVLTQQYQVLRTD

SEQ ID NO: 18 - protein, 30 aa, Artificial Sequence
  Cytoplasmic tail (R+13)
NRLTAFINDKLNIIHAMVLTQQYQVLRTDE

SEQ ID NO: 19 - protein, 31 aa, Artificial Sequence
  Cytoplasmic tail (R+14)
NRLTAFINDKLNIIHAMVLTQQYQVLRTDEE

SEQ ID NO: 20 - protein, 32 aa, Artificial Sequence
  Cytoplasmic tail (R+15)
NRLTAFINDKLNIIHAMVLTQQYQVLRTDEEA

SEQ ID NO: 21 - protein, 33 aa, Artificial Sequence
  Cytoplasmic tail (R+16)
NRLTAFINDKLNIIHAMVLTQQYQVLRTDEEAQ

SEQ ID NO: 22 - protein, 17 aa, Artificial Sequence
  Full R peptide only
VLTQQYQVLRTDEEAQD

SEQ ID NO: 23 - protein, 563 aa, Artificial Sequence
  Full Protein with signal sequence
MGFTTKIIFLYNLVLVYAGFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 24 - protein, 545 aa, Artificial Sequence
  Full protein without signal sequence
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 25 - protein, 358 aa, Artificial Sequence
  Surface protein subunit
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKR

SEQ ID NO: 26 - protein, 170 aa, Artificial Sequence
  Transmembrane protein subunit (without R peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 27 - protein, 187 aa, Artificial Sequence
  Transmembrane protein subunit (including R peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQD

SEQ ID NO: 28 - protein, 528 aa, Artificial Sequence
  BaEv envelope glycoprotein R-less
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAM

SEQ ID NO: 29 - protein, 529 aa, Artificial Sequence
  BaEv envelope glycoprotein R+1
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMV

SEQ ID NO: 30 - protein, 530 aa, Artificial Sequence
  BaEv envelope glycoprotein R+2
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVL

SEQ ID NO: 31 - protein, 531 aa, Artificial Sequence
  BaEv envelope glycoprotein R+3
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLT

SEQ ID NO: 32 - protein, 532 aa, Artificial Sequence
  BaEv envelope glycoprotein R+4
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQ

SEQ ID NO: 33 - protein, 533 aa, Artificial Sequence
  BaEv envelope glycoprotein R+5
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQ

SEQ ID NO: 34 - protein, 534 aa, Artificial Sequence
  BaEv envelope glycoprotein R+6
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQY

SEQ ID NO: 35 - protein, 535 aa, Artificial Sequence
  BaEv envelope glycoprotein R+7
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQ

SEQ ID NO: 36 - protein, 536 aa, Artificial Sequence
  BaEv envelope glycoprotein R+8
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQV

SEQ ID NO: 37 - protein, 537 aa, Artificial Sequence
  BaEv envelope glycoprotein R+9
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVL

SEQ ID NO: 38 - protein, 538 aa, Artificial Sequence
  BaEv envelope glycoprotein R+10
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLR

SEQ ID NO: 39 - protein, 539 aa, Artificial Sequence
  BaEv envelope glycoprotein R+11
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRT

SEQ ID NO: 40 - protein, 540 aa, Artificial Sequence
  BaEv envelope glycoprotein R+12
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTD

SEQ ID NO: 41 - protein, 541 aa, Artificial Sequence
  BaEv envelope glycoprotein R+13
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDE

SEQ ID NO: 42 - protein, 542 aa, Artificial Sequence
  BaEv envelope glycoprotein R+14
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEE

SEQ ID NO: 43 - protein, 543 aa, Artificial Sequence
  BaEv envelope glycoprotein R+15
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEA

SEQ ID NO: 44 - protein, 544 aa, Artificial Sequence
  BaEv envelope glycoprotein R+16
GFDDPRKAIELVQKRYGRPCDCSGGQVSEPPSDRVSQVTCSGKTAYLMPDQRWKCKSIPKDTSPSGPLQECPCNSYQSSVHSSCYTSYQQCRSGNKTYYTATLLKTQTGGTSDVQVLGSTNKLIQSPCNGIKGQSICWSTTAPIHVSDGGGPLDTTRIKSVQRKLEEIHKALYPELQYHPLAIPKVRDNLMVDAQTLNILNATYNLLLMSNTSLVDDCWLCLKLGPPTPLAIPNFLLSYVTRSSDNISCLIIPPLLVQPMQFSNSSCLFSPSYNSTEEIDLGHVAFSNCTSITNVTGPICAVNGSVFLCGNNMAYTYLPTNWTGLCVLATLLPDIDIIPGDEPVPIPAIDHFIYRPKRAIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQ

SEQ ID NO: 45 - protein, 171 aa, Artificial Sequence
  Transmembrane protein subunit (R+1 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMV

SEQ ID NO: 46 - protein, 172 aa, Artificial Sequence
  Transmembrane protein subunit (R+2 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVL

SEQ ID NO: 47 - protein, 173 aa, Artificial Sequence
  Transmembrane protein subunit (R+3 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLT

SEQ ID NO: 48 - protein, 174 aa, Artificial Sequence
  Transmembrane protein subunit (R+4 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQ

SEQ ID NO: 49 - protein, 175 aa, Artificial Sequence
  Transmembrane protein subunit (R+5 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQ

SEQ ID NO: 50 - protein, 176 aa, Artificial Sequence
  Transmembrane protein subunit (R+6 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQY

SEQ ID NO: 51 - protein, 177 aa, Artificial Sequence
  Transmembrane protein subunit (R+7 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQ

SEQ ID NO: 52 - protein, 178 aa, Artificial Sequence
  Transmembrane protein subunit (R+8 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQV

SEQ ID NO: 53 - protein, 179 aa, Artificial Sequence
  Transmembrane protein subunit (R+9 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVL

SEQ ID NO: 54 - protein, 180 aa, Artificial Sequence
  Transmembrane protein subunit (R+10 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLR

SEQ ID NO: 55 - protein, 181 aa, Artificial Sequence
  Transmembrane protein subunit (R+11 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRT

SEQ ID NO: 56 - protein, 182 aa, Artificial Sequence
  Transmembrane protein subunit (R+12 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTD

SEQ ID NO: 57 - protein, 183 aa, Artificial Sequence
  Transmembrane protein subunit (R+13 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDE

SEQ ID NO: 58 - protein, 184 aa, Artificial Sequence
  Transmembrane protein subunit (R+14 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEE

SEQ ID NO: 59 - protein, 185 aa, Artificial Sequence
  Transmembrane protein subunit (R+15 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEA

SEQ ID NO: 60 - protein, 186 aa, Artificial Sequence
  Transmembrane protein subunit (R+16 peptide)
AIQFIPLLAGLGITAAFTTGATGLGVSVTQYTKLSNQLISDVQILSSTIQDLQDQVDSLAEVVLQNRRGLDLLTAEQGGICLALQEKCCFYVNKSGIVRDKIKTLQEELERRRKDLASNPLWTGLQGLLPYLLPFLGPLLTLLLLLTIGPCIFNRLTAFINDKLNIIHAMVLTQQYQVLRTDEEAQ

SEQ ID NO: 61 - protein, 245 aa, Artificial Sequence
  Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 62 - protein, 242 aa, Artificial Sequence
  Anti-CD19 scFv (FMC63)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 63 - protein, 12 aa, Artificial Sequence
  IgG4 Hinge
ESKYGPPCPPCP

SEQ ID NO: 64 - protein, 25 aa, Artificial Sequence
  CD8 Hinge
TTTPAPRPPTPAPTIASQPLSLRPE

SEQ ID NO: 65 - protein, 39 aa, Artificial Sequence
  CD28
IEVMYPPPYLDNEKSNGTIIHVKGKHLCPSPLFPGPSKP

SEQ ID NO: 66 - protein, 44 aa, Artificial Sequence
  CD8
ACRPAAGGAVHTRGLDFACDIYIWAPLAGTCGVLLLSLVITLYC

SEQ ID NO: 67 - protein, 27 aa, Artificial Sequence
  CD28
FWVLVVVGGVLACYSLLVTVAFIIFWV

SEQ ID NO: 68 - protein, 27 aa, Artificial Sequence
  CD28
FWVLVVVGGVLACYSLLVTVAFIIFWV

SEQ ID NO: 69 - protein, 41 aa, Artificial Sequence
  CD28
RSKRSRLLHSDYMNMTPRRPGPTRKHYQPYAPPRDFAAYRS

SEQ ID NO: 70 - protein, 42 aa, Artificial Sequence
  4-1BB
KRGRKKLLYIFKQPFMRPVQTTQEEDGCSCRFPEEEEGGCEL

SEQ ID NO: 71 - protein, 112 aa, Artificial Sequence
  CD3zeta
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 72 - protein, 112 aa, Artificial Sequence
  CD3zeta
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR